(19) United States
(12) Patent Application Publication
(10) Pub. No.: US 2009/0275648 A1
(43) Pub. Date: Nov. 5, 2009

(54) MACROCYCLIC GHRELIN RECEPTOR ANTAGONISTS AND INVERSE AGONISTS AND METHODS OF USING THE SAME

(76) Inventors: Graeme L. Fraser, Rixensart (BE); Hamid R. Hoveyda, Bruxelles (BE)

Correspondence Address: MYERS BIGEL SIBLEY & SAJOVEC, PO BOX 37428, RALEIGH, NC, 27627, US

(21) Appl. No.: 11/577,922

(22) PCT Filed: Apr. 26, 2006
(86) PCT No.: PCT/US06/15698

§ 371 Date: Jul. 15, 2009

(30) Foreign Application Priority Data
Jun. 13, 2005 (US) PCT/US05/20887

(51) Int. Cl. A61K 31/395; C07D 269/00; A61K 31/352; A61P 3/04; A61P 3/10; A61P 29/00; A61P 9/00; A61P 1/00; A61P 1/16

(52) U.S. Cl. 514/454; 540/455; 540/456; 514/183

(57) ABSTRACT

The present invention provides novel conformationally-defined macrocyclic compounds that have been demonstrated to be selective modulators of the ghrelin receptor (growth hormone secretagogue receptor, GHS-R1a and subtypes, isoforms and/or variants thereof). Methods of synthesizing the novel compounds are also described herein. These compounds are useful as antagonists or inverse agonists of the ghrelin receptor and as medicaments for treatment and prevention of a range of medical conditions including, but not limited to, metabolic and/or endocrine disorders, cardiovascular disorders, obesity and obesity-associated disorders, gastrointestinal disorders, genetic disorders, hyperproliferative disorders and inflammatory disorders.

### RELATED APPLICATION INFORMATION

This application claims priority to International Application No. PCT/US2005/020887, filed Jun. 13, 2005, the disclosure of which is incorporated by reference herein in its entirety.

### FIELD OF THE INVENTION

The present invention relates to novel conformationally-defined macrocyclic compounds that have been demonstrated to function as modulators, in particular antagonists or inverse agonists, of the ghrelin (growth hormone secretagogue) receptor (GHS-R1a). The invention also relates to intermediates of these compounds, pharmaceutical compositions containing these compounds and methods of using the compounds. These novel macrocyclic compounds are useful as therapeutics for a range of indications including metabolic and/or endocrine disorders, cardiovascular disorders, obesity and obesity-associated disorders, gastrointestinal disorders, genetic disorders, hyperproliferative disorders and inflammatory disorders.

### BACKGROUND OF THE INVENTION

The improved understanding of various physiological regulatory pathways enabled through the research efforts in genomics and proteomics has begun to impact the discovery of novel pharmaceutical agents. In particular, the identification of key receptors and their endogenous ligands has created new opportunities for exploitation of these receptor/ligand pairs as therapeutic targets. For example, ghrelin is a recently characterized 28-amino acid peptide hormone that has been shown to mediate a variety of important physiological functions. (Kojima, M.; Hosoda, H. et al. Nature 1999, 402, 656-660). A novel characteristic of the structure is the presence of an n-octanoyl group on Ser³ that appears to be relevant to ghrelin's activity. This peptide has been demonstrated to be the endogenous ligand for a previously orphan G protein-coupled receptor (GPCR), type 1 growth hormone secretatogue receptor (hGHS-R1a). (Howard, A. D.; Feighner, S. D.; Cully, D. F.; Arena, J. P.; Liberator, P. A.; Rosenblum, C. I.; Hamelin, M.; Hreniuk, D. L.; Palyha, O. C.; Anderson, J.; Paress, P. S.; Diaz, C.; Chou, M.; Liu, K. K.; McKee, K. K.; Pong, S.-S.; Chaung, L. Y.; Elbrecht, A.; Dashkevicz, M.; Heavens, R.; Rigby, M.; Sirinathsinghji, D. J. S.; Dean, D. C.; Melillo, D. G.; Patchett, A. A.; Nargund, R.; Griffin, P. R.; DeMartino, J. A.; Gupta, S. K.; Schaeffer, J. M.; Smith, R. G.; Van der Ploeg, L. H. T. A receptor in pituitary and hypothalamus that functions in growth hormone release. Science 1996, 273, 974-977). GHS-R1a has recently been reclassified as the ghrelin receptor in recognition of its endogenous ligand (Davenport, A. P.; et al. International Union of Pharmacology. LVI. Ghrelin Receptor Nomenclature, Distribution, and Function. Pharmacol. Rev. 2005, 57, 541-546).

Even prior to the isolation of this receptor and its endogenous peptide ligand, a significant amount of research was devoted to finding agents that can stimulate growth hormone (GH) secretion. The proper regulation of human GH has importance not only for proper body growth, but also for a range of other critical physiological effects. GH and other GH-stimulating peptides, such as growth hormone-releasing hormone (GHRH) and growth hormone releasing factor (GRF), as well as their derivatives and analogues, are administered via injection. Therefore, to better take advantage of these positive effects, attention was focused on the development of orally active therapeutic agents that would increase GH secretion, termed GH secretagogues (GHS). Additionally, use of these agents was expected to be able to more closely mimic the pulsatile physiological release of GH.

Beginning with the identification of the growth hormone-releasing peptides (GHRP) in the late 1970's (Bowers, C. Y. Growth hormone-releasing peptides: physiology and clinical applications. Curr. Opin. Endocrinol. Diabetes 2000, 7, 168-174; Camanni, F.; Ghigo, E.; Arvat, E. Growth hormone-releasing peptides and their analogs. Front. Neurosci. 1998, 19, 47-72; Locatelli, V.; Torsello, A. Growth hormone secretagogues: focus on the growth hormone-releasing peptides. Pharmacol. Res. 1997, 36, 415-423). a host of agents have been studied for their potential to act as GHS. In addition to their stimulation of GH release and concomitant positive effects in that regard, GHS were projected to have utility in a variety of other disorders, including the treatment of wasting conditions (cachexia) as seen in HIV patients and cancer-induced anorexia, musculoskeletal frailty in the elderly, and growth hormone deficient diseases. Many efforts over the past 25 years have yielded a number of potent, orally available GHS (Isidro, M. L.; Cordido, F. Growth hormone secretagogues. Comb. Chem. High Throughput Screen. 2006, 9, 178-180; Smith, R. G.; Sun, Y. X.; Beatancourt, L.; Asnicar, M. Growth hormone secretagogues: prospects and pitfalls. Best Pract. Res. Clin. Endocriniol. Metab. 2004, 18, 333-347; Fehrentz, J.-A.; Martinez, J.; Boeglin, D.; Guerlavais, V.; Deghenghi, R. Growth hormone secretagogues: Past, present and future. IDrugs 2002, 5, 804-814; Svensson, J. Exp. Opin. Ther. Patents 2000, 10, 1071-1080; Nargund, R. P.; Patchett, A. A.; Bach, M. A.; Murphy, M. G.; Smith, R. G. Peptidomimetic growth hormone secretagogues. Design considerations and therapeutic potential. J. Med. Chem. 1998, 41, 3103-3127; Ghigo, E; Arvat, E.; Camanni, F. Orally active growth hormone secretagogues: state of the art and clinical perspective. Ann. Med. 1998, 30, 159-168). These include small peptides, such as hexarelin (Zentaris) and ipamorelin (Novo Nordisk), and adenosine analogues, as well as small molecules such as capromorelin (Pfizer), L-252,564 (Merck), MK-0677 (Merck), NN703 (tabimorelin, Novo Nordisk), G-7203 (Genentech), S-37435 (Kaken) and SM-130868 (Sumitomo). However, clinical tests with such agents have rendered disappointing results due to, among other things, lack of efficacy over prolonged treatment or undesired side effects, including irreversible inhibition of cytochrome P450 enzymes (Zdravkovic M.; Olse, A. K.; Christiansen, T.; et al. Eur. J. Clin. Pharmacol. 2003, 58, 683-688).

The cloning of the human receptor, which was actually enabled through the use of a synthetic GHS, and the subsequent identification of ghrelin have opened a variety of new chemical areas for investigation on both agonists and antagonists (Carpino, P. A. Exp. Opin. Ther Patents 2002, 12, 1599-1618). In particular, the ghrelin peptide has been found to have multiple other physiological functions apart from the stimulation of GH release, including regulation of food intake and appetite, promotion of weight gain, control of energy balance, and modulation of gastrointestinal (GI) motility and gastric acid secretion. The hormone has also been linked to control of glucose homeostasis, circadian rhythm and memory. (Van der Lely, A. J.; Tschop, M.; Heiman, M. L.; Ghigo, E. Biological, physiological, pathophysiological, and pharmacological aspects of ghrelin. Endocrine Rev. 2004, 25, 426-457; Inui, A.; Asakawa, A.; Bowers, C. Y.; Mantovani, G.; Laviano, A.; Meguid, M. M.; Fujimiya, M. Ghrelin, appetite, and gastric motility: the emerging role of the stomach as an endocrine organ. FASEB J. 2004, 18, 439-456; Diano, S. Farr, S. A.; Benoit, S. C.; et al. Ghrelin controls hippocampal spine synapse density and memory performance. Nat. Neuroscience 2006, 9, 381-388). Due to these myriad physiological effects, modulation of the ghrelin receptor has come under increasing study for therapeutic indications apart from those related to the GH secretory function (Dodge, J. A.; Heiman, M. L. Ghrelin receptor modulators. Ann. Rep. Med. Chem. 2003, 38, 81-88). For example, Intl. Pat. Appl. WO 2006/009645 and WO 2006/009674 describe the use of macrocyclic compounds as ghrelin modulators for use in the treatment of gastrointestinal (GI) disorders. Similarly, WO 2006/020930 and WO 2006/023608 describe structurally distinct ghrelin agonists (growth hormone secretagogues) for use in such GI disorders. In addition, Intl. Pat. Appl. WO 2004/09124 and WO 2005/68639 describe modified virus particles derived from short peptide sequences from the N-terminus of ghrelin that can be used as vaccines for treatment of obesity. Another vaccine approach for obesity is described in WO 2004/024183.

Not surprisingly due to the role of ghrelin in the control of appetite and feeding, particular interest has also been sparked in the development of ghrelin antagonists and inverse agonists as new anti-obesity pharmaceutical agents, as indeed has modulation of a number of peptide hormones and their receptors. (Spanswick, D.; Lee, K. Emerging antiobesity drugs. Exp. Opin. Emerging Drugs 2003, 8, 217-237; Horvath, T. L.; Castañeda, T.; Tang-Christensen, M.; Pagotto, U.; Tschöp, M. H. Ghrelin as a potential anti-obesity target. Curr. Pharm. Design 2003, 9, 1383-1395; Crowley, V. E. F.; Yeo, G. S. H.; O-Rahilly, S. Obesity therapy: altering the energy intake-and-expenditure balance sheet. Nat. Rev. Drug Disc. 2002, 1, 276-286). In contrast to ghrelin agonists, with the precedence in the search for GHS, the field of research on ghrelin antagonists and inverse agonists is significantly less mature. U.S. Published Patent Application 2003/0211967 and WO 01/87335 address the use of ghrelin antagonists as treatment for a variety of disease states including obesity and related disorders. Similarly, WO 01/56592 and US 2001/020012 describe the use of antagonists for the regulation of food intake. Likewise, WO 2004/004772 describes the use of GHS-R antagonists as a treatment for diabetes, obesity and appetite control. Their use for treatment of intestinal inflammation has also been described (WO 2004/084943). However, no specific examples of compounds, apart from ghrelin peptide and its analogues, for this purpose are presented in these applications. More recently, oxadiazole ghrelin antagonists have been reported which are also claimed to be effective in improving cognition, memory and other CNS disorders (WO 2005/112903).

Ghrelin antagonists and inverse agonists have also been considered for playing a role in the reduction of the incidence of the following obesity-associated conditions including diabetes, complications due to diabetes such as retinopathy, cardiovascular diseases, hypertension, dyslipidemia, osteoarthritis and certain forms of cancer. Indeed, in addition to the anti-obesity effects seen in animal studies, transgenic rats engineered without the GHS-R1a receptor have exhibited reduced food intake, diminished fat deposition, and decreased weight. However, the hormone's involvement in a number of physiological processes, including regulation of cardiovascular function and stress responses as well as growth hormone release, may indicate potential drawbacks to this strategy. Hence, complete lack of ghrelin may not be desirable, but suppression may be sufficient to control obesity. It should be noted that recent studies with ghrelin knockout mice, reveal that these mice do not exhibit the expected modifications in size and food intake among other physiological characteristics. (Sun, Y.; Ahmed, S.; Smith, R. G. Deletion of ghrelin impairs neither growth nor appetite. Mol. Cell. Biol. 2003, 23, 7973-7981; Wortley, K. E.; Anderson, K. D.; Garcia, K.; et al. Genetic deletion of ghrelin does not decrease food intake but influences metabolic fuel preferences. Proc. Natl. Acad. Sci. USA 2004, 101, 8227-8232).

Recently, BIM-28163 has been reported to function as an antagonist at the GHS-R1a receptor and inhibit receptor activation by native ghrelin. However, this same molecule is a full agonist with respect to stimulating weight gain and food intake. This and related peptidic ghrelin analogues effectively separate the GH-modulating activity of ghrelin from the effects of the peptide on weight gain and appetite. (Halem, H. A.; Taylor, J. E.; Dong, J. Z.; Shen, Y.; Datta, R.; Abizaid, A.; Diano, S.; Horvath, T.; Zizzari, P.; Bluet-Pajot, M.-T.; Epelbaum, J.; Culler, M. D. Novel analogs of ghrelin: physiological and clinical implications. Eur. J. Endocrinol. 2004, 151, S71-S75). Analogously, the macrocyclic ghrelin agonists described in WO 2006/009645 and WO 2006/009674 report the separation of the GI effects from the GH-release effects in animal models. Such separation shows that the involvement of ghrelin and its receptors in metabolism may be more complicated than originally thought.

Prader-Willi syndrome, the most common form of human syndromic obesity, is characterized paradoxically by GH deficiency and high ghrelin levels not decreased after feeding. (Cummings, D. E.; Clement, K.; Purnell, J. Q.; Vaisse, C.; Foster, K. E.; Frayo, R. S.; Schwartz, M. W.; Basdevant, A.; Weigle, D. S. Elevated ghrelin plasma levels in Prader-Willi syndrome. Nat. Med. 2002, 8, 643-644). Antagonists could have a role in treating this syndrome as well. Similarly, such agents may have potential for diabetic hyperphagia. Hyperphagia and altered fuel metabolism result from uncontrolled diabetes mellitus in humans. This has been suggested to occur through a combination of elevated ghrelin levels and decreased leptin through the NPY/AGRP pathway. Although levels of ghrelin are essentially the same in healthy and diabetic subjects, the different levels of ghrelin in diabetic hyperphagia could make it difficult to remain on diet therapies and an antagonist could be useful in assisting control. (Ishii, S.; Karnegai, J.; Tamura, H.; Shimizu, T.; Sugihara, H.; Oikawa, S. Role of ghrelin in streptozotocin-induced diabetic hyperphagia. Endocrinology 2002, 143, 4934-4937; Sindelar, D. K., Mystkowski, P., Marsh, D. J., Palmiter, R. D.; Schwartz, M. W Attenuation of diabetic hyperphagia in neuropeptide Y-deficient mice. Diabetes 2002, 51, 778-783).

Ghrelin levels are elevated in cirrhosis and with complications from chronic liver disease, although unlike levels of insulin-like growth factor-1 (IGF-1), they do not correlate to liver function. (Tacke, F.; Brabant, G.; Kruck, E.; Horn, R.; Schoffski, P.; Hecker, H.; Maims, M. P.; Trautwein, C. Ghrelin in chronic liver disease. J. Hepatology 2003, 38, 447-454). Ghrelin antagonists could be useful in controlling these liver diseases. Further, ghrelin and its receptor are overexpressed in numerous cancers. Antagonists would have potential application to treatment of cancer. Intl. Pat Appl. Publ. WO 02/90387 has described the use of interventionist strategies targeting GHS-R1a as an approach to treatment of cancers of the reproductive system.

Despite the interest in ghrelin antagonists, only a limited number of small molecule ghrelin antagonists have yet been reported in the patent or scientific literature including diaminopyrimidines, tetralin carboxamides, isoxazole carboxamides, β-carbolines and oxadiazoles. (U.S. Pat. Appl. Publ. US 2005/0171131; US 2005/0171132; Intl. Pat. Appl. Publ. WO 2005/030734; WO 2005/112903; WO 2005/48916; Zhao, H.; Xin, Z.; Liu, G.; Schaefer, V. G.; Falls, H. D.; Kaszubska, W.; Colins, C. A.; Sham, H. L. J. Med. Chem. 2004, 47, 6655-6657; Xin, Z.; Zhao, H.; Serby, M. D.; Liu, B.; Schaefer, V. G.; Falls, H. D.; Kaszubska, W.; Colins, C. A.; Sham, H. L.; Liu, G. Bioorg. Med. Chem. Lett. 2005, 15, 1201-1204; Zhao, H.; Xin, Z.; Patel, J. R., Nelson, T. J.; Liu, B.; Szczepankiewicz, B. G.; Schaefer, V. G.; Falls, H. D.; Kaszubska, W.; Collins, C. A.; Sham, H. L.; Liu, G. Bioorg. Med. Chem. Lett. 2005, 15, 1825-1828; Liu, B.; Liu, G.; Xin, Z.; Serby, M. D.; Zhao, H.; Schaefer, V. G.; Falls, H. D.; Kaszubska, W.; Collins, C. A.; Sham, H. L. Bioorg. Med. Chem. Lett. 2004, 14, 5223-5226). WO 2005/114180 describes a number of individual compounds containing heteroaryl core structures, such as isoazoles, 1,2,4-oxadiazoles and 1,2,4-triazoles, as “functional ghrelin antagonists” and their uses as therapeutic agents for the treatment of obesity and diabetes. Other heterocyclic structures, some of which displayed antagonist activity, are reported in WO 2005/035498, WO 2005/097788 and US 2005/0187237. The remaining known ghrelin antagonists are primarily peptidic in nature (WO 2004/09616, WO 02/08250, WO 03/04518, US 2002/0187938, Pinilla, L.; Barreiro, M. L.; Tena-Sempere, M.; Aguilar E. Neuroendocrinology 2003, 77, 83-90) although antagonists based on nucleic acids have also been disclosed (WO 2004/013274; WO 2005/49828; Helmling, S.; Maasch, C.; Eulberg, D.; et al. Inhibition of ghrelin action in vitro and in vivo by an RNA-Spiegelmer. Proc. Natl. Acad. Sci. USA 2004, 101, 13174-13179; Shearman, L. P.; Wang, S. P.; Helmling, S.; et al. Ghrelin neutralization by a ribonucleic acid-SPM ameliorates obesity in diet-induced obese mice. Endocrinology 2006, 147, 1517-1526). The compounds of the present invention are structurally distinct from all of these previously reported ghrelin antagonist structures.

The 14-amino acid compound, vapreotide, a small somatostatin mimetic, was demonstrated to be a ghrelin antagonist. (Deghenghi R, Papotti M, Ghigo E, Muccioli G, Locatelli V. Somatostatin octapeptides (lanreotide, octreotide, vapreotide, and their analogs) share the growth hormone-releasing peptide receptor in the human pituitary gland. Endocrine 2001, 14, 29-33). The binding activity of analogues of the cyclic neuropeptide cortistatin to the growth hormone secretatogue receptor has been disclosed (WO 03/004518). These compounds exhibit an IC₅₀ of 24-33 nM. In particular, one of these analogues, EP-01492 (cortistatin 8) has been advanced into preclinical studies for the treatment of obesity as a ghrelin antagonist. (Deghenghi R, Broglio F, Papotti M, et al. Targeting the ghrelin receptor—Orally active GHS and cortistatin analogs. Endocrine 2003, 22, 13-18; Sibilia, V.; Muccioli, G.; Deghenghi, R.; Pagani, F.; DeLuca, V.; Rapetti, D.; Locatelli, V.; Netti, C. Evidence for a role of the GHS-R1a receptor in ghrelin inhibition of gastric acid secretion in the rat. J. Neuroendocrinol. 2006, 18, 122-128).

A limited series of peptides as ghrelin antagonists containing the very specific short octanoylated sequence known to be critical for binding to GHS-R1a has been reported (U.S. Pat. Appl. No. 2002/0187938; Intl. Pat. Appl. No. WO 02/08250). Action of [D-Lys³]-GHRP-6 has been described as a ghrelin antagonist. (Pinilla, L.; Barreiro, M. L.; Tena-Sempere, M.; Aguilar E. Neuroendocrinology 2003, 77, 83-90) More recently, the substance P peptide derivative, L-756,867 (EP-80317, [D-Arg¹,D-Phe⁵,D-Trp^{7,9},Leu¹¹]-substance P), a weak ghrelin antagonist, was demonstrated to be a potent inverse agonist (K_{d/i}=45 nM) to open another potential approach to the treatment of obesity targeting the ghrelin receptor. (Holst, B.; Schwartz, T. W. Constitutive ghrelin receptor activity as a signaling set-point in appetite regulation. Trends Pharmacol. Sci. 2004, 25, 113-117; Holst, B.; Cygankiewicz, A.; Jensen, T. H.; Ankersen, M.; Schwartz, T. W. High constitutive signaling of the ghrelin receptor-identification of a potent inverse agonist. Mol. Endocrinol. 2003, 17, 2201-2210; Cheng, K.; Wei, L.; Chaung, L.-Y.; et al. Inhibition of L-692,429-stimulated rat growth hormone release by a weak substance P antagonist, L-756,867. J. Endocrinol. 1997, 152, 155-158). However, the use of this particular agent may be limited due to its poor selectivity since it also interacts at the neurokinin-1 and bombesin receptors.

The use of inverse agonists has been suggested to even be of more relevant use for the control of appetite due to the high constitutive activity of the ghrelin receptor. (Holst, B.; Holliday, N. D.; Bach, A.; Elling, C. E.; Cox, H. M.; Schwartz, T. W. Common structural basis for constitutive activity of the ghrelin receptor family. J. Biol. Chem. 2004, 279, 53806-53817). However, apart from the L-756,867 peptide and a single pyrrole compound, TM27810, (WO 2004/056869), no inverse agonists have yet been reported. The compounds of the present invention therefore constitute the first series of ghrelin inverse agonist molecules identified.

In fact, it has been argued that it is actually beneficial to have compounds that act as both ghrelin receptor antagonists and inverse agonists in order to best control feeding (Holst, B. Schwartz, T. Ghrelin receptor mutations—too little height and too much hunger. J. Clin. Invest. 2006, 116, 637-641). The recent observation that humans possessing a mutation in the ghrelin receptor that impairs constitutive activity are of short stature illustrates the importance of the constitutive activity to the normal in vivo function of this receptor. (Pantel, J.; Legendre, M. Cabrol, S.; et al. Loss of constitutive activity of the growth hormone secretagogue receptor in familial short stature. J. Clin. Invest. 2006, 116, 760-768). As shown in the Examples, some compounds of the present invention act as both ghrelin receptor antagonists and inverse agonists.

Accordingly, with so few examples of ghrelin antagonists or inverse agonists suitable for pharmacological intervention, there is a need for additional compounds that modulate the ghrelin receptor and suppress ghrelin release.

### SUMMARY OF THE INVENTION

The present invention provides novel conformationally-defined macrocyclic compounds that can function as modulators, in particular antagonists or inverse agonists, of the ghrelin (growth hormone secretagogue) receptor (GHS-R1a).

According to aspects of the present invention, the present invention relates to compounds according to formula (I):

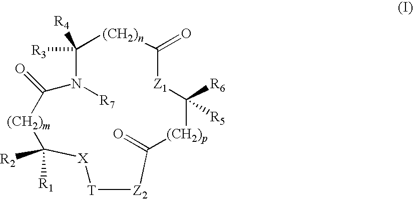

or an optical isomer, enantiomer, diastereomer, racemate or stereochemical mixture thereof, wherein:

X is NR₁₃, wherein R₁₃ is hydrogen, C₁-C₄ alkyl or R₁₃ and R₂ together form a 3-, 4-, 5-, 6- or 7-membered heterocyclic ring, wherein the ring optionally comprises an O, S or additional N atom in the ring and is optionally substituted with R₈ as defined below;

Z₁ is NR₁₁, wherein R₁₁ is hydrogen, C₁₋₄ alkyl or R₁₁ and R₃ together form a 4-, 5-, 6-, 7- or 8-membered heterocyclic ring, wherein the ring optionally comprises an O, S or additional N atom in the ring and is optionally substituted with R₈ as defined below;

Z₂ is NH;

m, n and p are each 0;

R₁ and R₆ are each independently hydrogen;

R₂ is —(CH₂)ₛCH₃, —CH(CH₃)(CH₂)ₜCH₃, —(CH₂)ᵤCH(CH₃)₂, —C(CH₃)₃, —(CH₂)ᵥ—R₁₄, —CH(OR₁₅)CH₃, cycloalkyl or substituted cycloalkyl wherein s is 1, 2, 3, 4 or 5; t is 1, 2 or 3; u is 0, 1, 2 or 3; and v is 0, 1, 2, 3 or 4; R₁₄ is aryl, substituted aryl, heteroaryl, substituted heteroaryl, cycloalkyl or substituted cycloalkyl; R₁₅ is hydrogen, alkyl, substituted alkyl, cycloalkyl, substituted cycloalkyl, heterocyclic, substituted heterocyclic, acyl, amino acyl, sulfonyl, carboxyalkyl, carboxyaryl, amido, aryl, substituted aryl, heteroaryl or substituted heteroaryl; or, alternatively, R₂ and R₁₃ together form a 3-, 4-, 5-, 6- or 7-membered heterocyclic ring, wherein the ring optionally comprises an O, S or additional N atom in the ring and is optionally substituted with R₈ as defined below;

R₃ and R₄ are each independently hydrogen or an amino acid side chain comprising —CH₃, —CH₂CH₃, —CH(CH₃)₂, —CR₁₇ₐR_{17b}(OR₁₆); or R₃ and R₄ together or R₃ and R₇ together form a 3-, 4-, 5-, 6- or 7-membered ring, respectively, optionally comprising an O or S atom in the ring and optionally substituted with R₈ as defined below; or R₃ and R₁₁ together form a 4-, 5-, 6-, 7- or 8-membered heterocyclic ring, wherein the ring optionally comprises an O, S or additional N atom in the ring and optionally substituted with R₈ as defined below;
wherein:
R₁₆ is hydrogen, alkyl, substituted alkyl, cycloalkyl, substituted cycloalkyl, heterocyclic, substituted heterocyclic, acyl, amino acyl, sulfonyl, carboxyalkyl, carboxyaryl, amido, aryl, substituted aryl, heteroaryl and substituted heteroaryl; and
R₁₇ₐ and R_{17b} are each independently hydrogen, —CH₃, —CH₂CH₃—CH(CH₃)₂ or —C(CH₃)₃;

R₅ is an amino acid side chain comprising —(CH₂)_{w}CH₃, —CH(CH₃)(CH₂)ₓCH₃, —(CH₂)_{y}CH(CH₃)₂, —C(CH₃)₃, —(CH₂)_{z1}—R₁₈ₐ, —(CR₁₁₀R₁₁₁)_{z2}—R_{18b} wherein w is 2, 3, 4 or 5; x is 1, 2 or 3; y is 0, 1, 2 or 3; z1 is 0, 1, 2, 3 or 4; and z2 is 0, 1 or 2; R₁₈ₐ and R_{18b} are each independently aryl, substituted aryl, heteroaryl, substituted heteroaryl, cycloalkyl and substituted cycloalkyl; R₁₁₀ and R₁₁₁ are each independently hydrogen C₁-C₄ alkyl, hydroxyl, amino or fluoro, with the proviso that at least one of R₁₁₀ and R₁₁₁ is not hydrogen;

R₇ is hydrogen, C₁-C₄ alkyl or R₇ and R₃ together form a 3-, 4-, 5-, 6- or 7-membered ring, respectively, optionally comprising an O or S atom in the ring and optionally substituted with R₈ as defined below;

R₈ is substituted for one or more hydrogen atoms on the 3-, 4-, 5-, 6- or 7-membered ring structure and is independently selected from the group consisting of alkyl, substituted alkyl, cycloalkyl, substituted cycloalkyl, heterocyclic, substituted heterocyclic, aryl, substituted aryl, heteroaryl, substituted heteroaryl, hydroxy, alkoxy, aryloxy, oxo, amino, halogen, formyl, acyl, carboxy, carboxyalkyl, carboxyaryl, amido, carbamoyl, guanidino, ureido, amidino, mercapto, sulfinyl, sulfonyl and sulfonamide, or, alternatively, R₈ is a fused cycloalkyl, a substituted fused cycloalkyl, a fused heterocyclic, a substituted fused heterocyclic group, a fused aryl, a substituted fused aryl, a heteroaryl or a substituted fused heteroaryl ring substituted for hydrogen atoms on two adjacent atoms; and

T is a bivalent radical of formula IV:

-U-(CH₂)_{d}-W-Y-Z-(CH₂)ₑ— (IV)
wherein d and e are each independently 0, 1, 2, 3, 4 or 5; Y and Z are each optionally present; U is —CR₂₁R₂₂—, or —C(═O)— and is bonded to X of formula I; W, Y and Z are each —O—, —NR₂₃—, —S—, —SO—, —SO₂—, —C(═O)—O—, —O—C(═O)—, —C(═O)—NH—, —NH—C(═O)—, —SO₂—NH—, —NH—SO₂—, —CR₂₄R₂₅—, —CH═CH— with the configuration Z or E, —C≡C—, or the ring structures below:

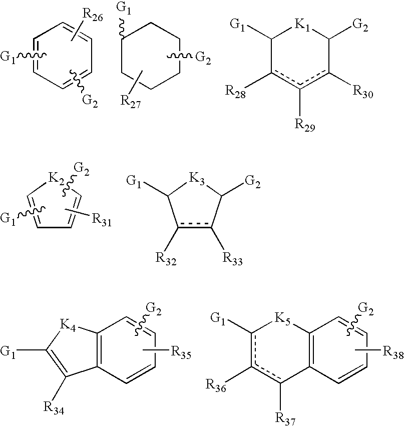

wherein G₁ and G₂ are each independently a covalent bond or a bivalent radical selected from the group consisting of —O—, —NR₃₉—, —S—, —SO—, —SO₂—, —C(═O)—, —C(═O)—O—, —O—C(═O)—, —C(═O)NH—, —NH—C(═O)—, —SO₂—NH—, —NH—SO₂—, —CR₄₀R₄₁—, —CH═CH— with the configuration Z or E, and —C≡C—; with G₁ being bonded closest to the group U; wherein any carbon atom in the rings not otherwise defined, is optionally replaced by N, with the proviso that the ring cannot contain more than four N atoms; K₁, K₂, K₃, K₄ and K₅ are each independently O, NR₄₂ or S, wherein R₄₂ is defined below;
R₂₁ and R₂₂ are each independently hydrogen, lower alkyl, or substituted lower alkyl, or R₂₁ and R₂₂ together form a 3- to 12-membered cyclic ring optionally comprising one or more heteroatoms selected from the group consisting of O, S and N, wherein the ring is optionally substituted with R₈ as defined previously;
R₂₃, R₃₉ and R₄₂ are each independently hydrogen, alkyl, substituted alkyl, cycloalkyl, substituted cycloalkyl, heterocyclic, substituted heterocyclic, aryl, substituted aryl, heteroaryl, substituted heteroaryl, formyl, acyl, carboxyalkyl, carboxyaryl, amido, amidino, sulfonyl or sulfonamido;
R₂₄ and R₂₅ are each independently hydrogen, lower alkyl, substituted lower alkyl, R_{AA}, wherein R_{AA} is a side chain of a standard or unusual amino acid, or R₂₄ and R₂₅ together form a 3- to 12-membered cyclic ring optionally comprising one or more heteroatoms selected from the group consisting of O, S and N; or one of R₂₄ or R₂₅ is hydroxy, alkoxy, aryloxy, amino, mercapto, carbamoyl, amidino, ureido or guanidino while the other is hydrogen, lower alkyl or substituted lower alkyl, except when the carbon to which R₂₄ and R₂₅ are bonded is also bonded to another heteroatom;
R₂₆ is optionally present and, when present, is substituted for one or more hydrogen atoms on the indicated ring and each is independently selected from the group consisting of halogen, trifluoromethyl, alkyl, substituted alkyl, cycloalkyl, substituted cycloalkyl, heterocyclic, substituted heterocyclic, aryl, substituted aryl, heteroaryl, substituted heteroaryl, hydroxy, alkoxy, aryloxy, amino, formyl, acyl, carboxy, carboxyalkyl, carboxyaryl, amido, carbamoyl, guanidino, ureido, amidino, cyano, nitro, mercapto, sulfinyl, sulfonyl and sulfonamido;
R₂₇ is optionally present and, when present, is substituted for one or more hydrogen atoms on the indicated ring and each is independently selected from the group consisting of alkyl, substituted alkyl, cycloalkyl, substituted cycloalkyl, heterocyclic, substituted heterocyclic, aryl, substituted aryl, heteroaryl, substituted heteroaryl, hydroxy, alkoxy, aryloxy, oxo, amino, formyl, acyl, carboxy, carboxyalkyl, carboxyaryl, amido, carbamoyl, guanidino, ureido, amidino, mercapto, sulfinyl, sulfonyl and sulfonamido;
R₂₈, R₂₉, R₃₀, R₃₂, R₃₃, R₃₄, R₃₆ and R₃₇ are each optionally present and when no double bond is present to the carbon atom to which it is bonded in the ring, two groups are optionally present, and, when present, is substituted for one hydrogen present in the ring, or when no double bond is present to the carbon atom to which it is bonded in the ring, is substituted for one or both of the two hydrogen atoms present on the ring and each is independently selected from the group consisting of alkyl, substituted alkyl, cycloalkyl, substituted cycloalkyl, heterocyclic, substituted heterocyclic, aryl, substituted aryl, heteroaryl, substituted heteroaryl, hydroxy, alkoxy, aryloxy, oxo, amino, formyl, acyl, carboxy, carboxyalkyl, carboxyaryl, amido, carbamoyl, guanidino, ureido, amidino, mercapto, sulfinyl, sulfonyl, sulfonamido and, only if a double bond is present to the carbon atom to which it is bonded, halogen;
R₃₁, R₃₅ and R₃₈ are each optionally present and, when present, are substituted for one or more hydrogen atoms on the indicated ring and each is independently selected from the group consisting of halogen, trifluoromethyl, alkyl, substituted alkyl, cycloalkyl, substituted cycloalkyl, heterocyclic, substituted heterocyclic, aryl, substituted aryl, heteroaryl, substituted heteroaryl, hydroxy, alkoxy, aryloxy, amino, formyl, acyl, carboxy, carboxyalkyl, carboxyaryl, amido, carbamoyl, guanidino, ureido, amidino, cyano, nitro, mercapto, sulfinyl, sulfonyl and sulfonamido; and
R₄₀ and R₄₁ are each independently hydrogen, lower alkyl, substituted lower alkyl, R_{AA} as defined above, or R₄₀ and R₄₁ together form a 3- to 12-membered cyclic ring optionally comprising one or more heteroatoms selected from the group consisting of O, S and N wherein the ring is optionally substituted with R as defined previously, or one of R₄₀ and R₄₁ is hydroxy, alkoxy, aryloxy, amino, mercapto, carbamoyl, amidino, ureido or guanidino, while the other is hydrogen, lower alkyl or substituted lower alkyl, except when the carbon to which R₄₀ and R₄₁ are bonded is also bonded to another heteroatom;
with the proviso that T is not an amino acid residue, dipeptide fragment, tripeptide fragment or higher order peptide fragment comprising standard amino acids.

Further aspects of the present invention provide pharmaceutical compositions comprising: (a) a compound of the present invention; and (b) a pharmaceutically acceptable carrier, excipient or diluent.

Additional aspects of the present invention provide kits comprising one or more containers containing pharmaceutical dosage units comprising an effective amount of one or more compounds of the present invention packaged with optional instructions for the use thereof.

In further aspects, the present invention provides methods of modulating GHS-R1a receptor activity in a mammal comprising administering an effective GHS-R1a receptor activity modulating amount of a compound of the present invention. According to some aspects of the present invention, the effective GHS-R1a receptor activity modulating amount of the compound does not result in a significant amount of growth hormone release. According to other aspects, the compound is a ghrelin receptor antagonist or a GHS-R1a receptor antagonist. In yet another aspect, the compound is a ghrelin receptor inverse agonist or a GHS-R1a receptor inverse agonist. According to another aspect of the present invention, the compound is both a ghrelin receptor antagonist and a ghrelin receptor inverse agonist or a GHS-R1a receptor antagonist and a GHS-R1a receptor inverse agonist.

Aspects of the present invention further relate to methods of preventing and/or treating disorders such as metabolic and/or endocrine disorders, cardiovascular disorders, genetic disorders, hyperproliferative disorders and inflammatory disorders.

Further aspects of the present invention relate to methods of making the compounds of formula I.

The present invention also relates to compounds of formula I useful for the preparation of a medicament for prevention and/or treatment of the disorders described herein.

The foregoing and other aspects of the present invention are explained in greater detail in the specification set forth below.

### BRIEF DESCRIPTION OF THE DRAWINGS

FIG. 1 shows a graph presenting results of a study to assess the functional activity of an exemplary compound of the present invention. FIG. 1(A) shows a graph depicting lack of agonist activity of the exemplary compound at the hGHS-R1a receptor. FIG. 1(B) shows a graph depicting antagonist activity at the hGHS-R1a receptor. The materials and methods for this study are described in further detail in Example 3.

FIG. 2 shows a chemical synthesis scheme for an exemplary compound of the present invention.

FIG. 3 shows a chemical synthesis scheme for another exemplary compound of the present invention.

### DETAILED DESCRIPTION

The foregoing and other aspects of the present invention will now be described in more detail with respect to other embodiments described herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise. Additionally, as used herein, the term “and/or” includes any and all combinations of one or more of the associated listed items and may be abbreviated as “/”.

Unless otherwise defined, all technical and scientific terms used herein have the same meaning as commonly understood by one of ordinary skill in the art to which this invention belongs.

All publications, U.S. patent applications, U.S. patents and other references cited herein are incorporated by reference in their entireties.

The term “alkyl” refers to straight or branched chain saturated or partially unsaturated hydrocarbon groups having from 1 to 20 carbon atoms, and in some instances, 1 to 8 carbon atoms. The term “lower alkyl” refers to alkyl groups containing 1 to 6 carbon atoms. Examples of alkyl groups include, but are not limited to, methyl, ethyl, isopropyl, tert-butyl, 3-hexenyl, and 2-butynyl. By “unsaturated” is meant the presence of 1, 2 or 3 double or triple bonds, or a combination of the two. Such alkyl groups may also be optionally substituted as described below.

When a subscript is used with reference to an alkyl or other hydrocarbon group defined herein, the subscript refers to the number of carbon atoms that the group may contain. For example, C₂-C₄ alkyl indicates an alkyl group that contains 2, 3 or 4 carbon atoms.

The term “cycloalkyl” refers to saturated or partially unsaturated cyclic hydrocarbon groups having from 3 to 15 carbon atoms in the ring, and in some instances, 3 to 7, and to alkyl groups containing said cyclic hydrocarbon groups. Examples of cycloalkyl groups include, but are not limited to, cyclopropyl, cyclopropylmethyl, cyclopentyl, 2-(cyclohexyl)ethyl, cycloheptyl, and cyclohexenyl. Cycloalkyl as defined herein also includes groups with multiple carbon rings, each of which may be saturated or partially unsaturated, for example decalinyl, [2.2.1]-bicycloheptanyl or adamantanyl. All such cycloalkyl groups may also be optionally substituted as described below.

The term “aromatic” refers to an unsaturated cyclic hydrocarbon group having a conjugated pi electron system that contains 4n+2 electrons where n is an integer greater than or equal to 1. Aromatic molecules are typically stable and are depicted as a planar ring of atoms with resonance structures that consist of alternating double and single bonds, for example benzene or naphthalene.

The term “aryl” refers to an aromatic group in a single or fused carbocyclic ring system having from 6 to 15 ring atoms, and in some instances, 6 to 10, and to alkyl groups containing said aromatic groups. Examples of aryl groups include, but are not limited to, phenyl, 1-naphthyl, 2-naphthyl and benzyl. Aryl as defined herein also includes groups with multiple aryl rings which may be fused, as in naphthyl and anthracenyl, or unfused, as in biphenyl and terphenyl. Aryl also refers to bicyclic or tricyclic carbon rings, where one of the rings is aromatic and the others of which may be saturated, partially unsaturated or aromatic, for example, indanyl or tetrahydronaphthyl (tetralinyl). All such aryl groups may also be optionally substituted as described below.

The term “heterocycle” or “heterocyclic” refers to saturated or partially unsaturated monocyclic, bicyclic or tricyclic groups having from 3 to 15 atoms, and in some instances, 3 to 7, with at least one heteroatom in at least one of the rings, said heteroatom being selected from O, S or N. Each ring of the heterocyclic group can contain one or two O atoms, one or two S atoms, one to four N atoms, provided that the total number of heteroatoms in each ring is four or less and each ring contains at least one carbon atom. The fused rings completing the bicyclic or tricyclic heterocyclic groups may contain only carbon atoms and may be saturated or partially unsaturated. The N and S atoms may optionally be oxidized and the N atoms may optionally be quaternized. Heterocyclic also refers to alkyl groups containing said monocyclic, bicyclic or tricyclic heterocyclic groups. Examples of heterocyclic rings include, but are not limited to, 2- or 3-piperidinyl, 2- or 3-piperazinyl, 2- or 3-morpholinyl. All such heterocyclic groups may also be optionally substituted as described below

The term “heteroaryl” refers to an aromatic group in a single or fused ring system having from 5 to 15 ring atoms, and in some instances, 5 to 10, which have at least one heteroatom in at least one of the rings, said heteroatom being selected from O, S or N. Each ring of the heteroaryl group can contain one or two O atoms, one or two S atoms, one to four N atoms, provided that the total number of heteroatoms in each ring is four or less and each ring contains at least one carbon atom. The fused rings completing the bicyclic or tricyclic groups may contain only carbon atoms and may be saturated, partially unsaturated or aromatic. In structures where the lone pair of electrons of a nitrogen atom is not involved in completing the aromatic pi electron system, the N atoms may optionally be quaternized or oxidized to the N-oxide. Heteroaryl also refers to alkyl groups containing said cyclic groups. Examples of monocyclic heteroaryl groups include, but are not limited to pyrrolyl, pyrazolyl, pyrazolinyl, imidazolyl, oxazolyl, isoxazolyl, thiazolyl, thiadiazolyl, isothiazolyl, furanyl, thienyl, oxadiazolyl, pyridyl, pyrazinyl, pyrimidinyl, pyridazinyl, and triazinyl. Examples of bicyclic heteroaryl groups include, but are not limited to indolyl, benzothiazolyl, benzoxazolyl, benzothienyl, quinolinyl, tetrahydroisoquinolinyl, isoquinolinyl, benzimidazolyl, benzopyranyl, indolizinyl, benzofuranyl, isobenzofuranyl, chromonyl, coumarinyl, benzopyranyl, cinnolinyl, quinoxalinyl, indazolyl, purinyl, pyrrolopyridinyl, furopyridinyl, thienopyridinyl, dihydroisoindolyl, and tetrahydroquinolinyl. Examples of tricyclic heteroaryl groups include, but are not limited to carbazolyl, benzindolyl, phenanthrollinyl, acridinyl, phenanthridinyl, and xanthenyl. All such heteroaryl groups may also be optionally substituted as described below.

The term “hydroxy” refers to the group —OH.

The term “alkoxy” refers to the group —ORₐ, wherein Rₐ is alkyl, cycloalkyl or heterocyclic. Examples include, but are not limited to methoxy, ethoxy, tert-butoxy, cyclohexyloxy and tetrahydropyranyloxy.

The term “aryloxy” refers to the group —OR_{b} wherein R_{b} is aryl or heteroaryl. Examples include, but are not limited to phenoxy, benzyloxy and 2-naphthyloxy.

The term “acyl” refers to the group C(═O)—R_{c} wherein R_{c} is alkyl, cycloalkyl, heterocyclic, aryl or heteroaryl. Examples include, but are not limited to, acetyl, benzoyl and furoyl.

The term “amino acyl” indicates an acyl group that is derived from an amino acid.

The term “amino” refers to an —NR_{d}Rₑ group wherein R_{d} and Rₑ are independently selected from the group consisting of hydrogen, alkyl, cycloalkyl, heterocyclic, aryl and heteroaryl. Alternatively, R_{d} and Rₑ together form a heterocyclic ring of 3 to 8 members, optionally substituted with unsubstituted alkyl, unsubstituted cycloalkyl, unsubstituted heterocyclic, unsubstituted aryl, unsubstituted heteroaryl, hydroxy, alkoxy, aryloxy, acyl, amino, amido, carboxy, carboxyalkyl, carboxyaryl, mercapto, sulfinyl, sulfonyl, sulfonamido, amidino, carbamoyl, guanidino or ureido, and optionally containing one to three additional heteroatoms selected from O, S or N.

The term “amido” refers to the group —C(═O)—NR_{f}R_{g} wherein R_{f} and R_{g} are independently selected from the group consisting of hydrogen, alkyl, cycloalkyl, heterocyclic, aryl and heteroaryl. Alternatively, R_{f} and R_{g} together form a heterocyclic ring of 3 to 8 members, optionally substituted with unsubstituted alkyl, unsubstituted cycloalkyl, unsubstituted heterocyclic, unsubstituted aryl, unsubstituted heteroaryl, hydroxy, alkoxy, aryloxy, acyl, amino, amido, carboxy, carboxyalkyl, carboxyaryl, mercapto, sulfinyl, sulfonyl, sulfonamido, amidino, carbamoyl, guanidino or ureido, and optionally containing one to three additional heteroatoms selected from O, S or N.

The term “amidino” refers to the group —C(═NRₕ)NRᵢRⱼ wherein Rₕ is selected from the group consisting of hydrogen, alkyl, cycloalkyl, heterocyclic, aryl and heteroaryl; and Rᵢ and Rⱼ are independently selected from the group consisting of hydrogen, alkyl, cycloalkyl, heterocyclic, aryl and heteroaryl. Alternatively, Rᵢ and Rⱼ together form a heterocyclic ring of 3 to 8 members, optionally substituted with unsubstituted alkyl, unsubstituted cycloalkyl, unsubstituted heterocyclic, unsubstituted aryl, unsubstituted heteroaryl, hydroxy, alkoxy, aryloxy, acyl, amino, amido, carboxy, carboxyalkyl, carboxyaryl, mercapto, sulfinyl, sulfonyl, sulfonamido, amidino, carbamoyl, guanidino or ureido, and optionally containing one to three additional heteroatoms selected from O, S or N.

The term “carboxy” refers to the group —CO₂H.

The term “carboxyalkyl” refers to the group —CO₂Rₖ, wherein Rₖ is alkyl, cycloalkyl or heterocyclic.

The term “carboxyaryl” refers to the group —CO₂Rₘ, wherein Rₘ is aryl or heteroaryl.

The term “cyano” refers to the group —CN.

The term “formyl” refers to the group —C(═O)H, also denoted —CHO.

The term “halo,” “halogen” or “halide” refers to fluoro, fluorine or fluoride, chloro, chlorine or chloride, bromo, bromine or bromide, and iodo, iodine or iodide, respectively.

The term “oxo” refers to the bivalent group ═O, which is substituted in place of two hydrogen atoms on the same carbon to form a carbonyl group.

The term “mercapto” refers to the group —SRₙ wherein Rₙ is hydrogen, alkyl, cycloalkyl, heterocyclic, aryl or heteroaryl.

The term “nitro” refers to the group —NO₂.

The term “trifluoromethyl” refers to the group —CF₃.

The term “sulfinyl” refers to the group —S(═O)Rₚ wherein Rₚ is alkyl, cycloalkyl, heterocyclic, aryl or heteroaryl.

The term “sulfonyl” refers to the group —S(═O)₂—R_{q1} wherein R_{q1} is alkyl, cycloalkyl, heterocyclic, aryl or heteroaryl.

The term “aminosulfonyl” refers to the group —NR_{q2}—S(═O)₂—R_{q3} wherein R_{q2} is hydrogen, alkyl, cycloalkyl, heterocyclic, aryl or heteroaryl; and R_{q3} is alkyl, cycloalkyl, heterocyclic, aryl or heteroaryl.

The term “sulfonamido” refers to the group —S(═O)₂—NRᵣRₛ wherein Rᵣ and Rₛ are independently selected from the group consisting of hydrogen, alkyl, cycloalkyl, heterocyclic, aryl or heteroaryl. Alternatively, Rᵣ and Rₛ together form a heterocyclic ring of 3 to 8 members, optionally substituted with unsubstituted alkyl, unsubstituted cycloalkyl, unsubstituted heterocyclic, unsubstituted aryl, unsubstituted heteroaryl, hydroxy, alkoxy, aryloxy, acyl, amino, amido, carboxy, carboxyalkyl, carboxyaryl, mercapto, sulfinyl, sulfonyl, sulfonamido, amidino, carbamoyl, guanidino or ureido, and optionally containing one to three additional heteroatoms selected from O, S or N.

The term “carbamoyl” refers to a group of the formula —N(Rₜ)—C(═O)—ORᵤ wherein Rₜ is selected from hydrogen, alkyl, cycloalkyl, heterocyclic, aryl or heteroaryl; and Rᵤ is selected from alkyl, cycloalkyl, heterocylic, aryl or heteroaryl.

The term “guanidino” refers to a group of the formula —N(Rᵥ)—C(═NR_{w})—NRₓR_{y} wherein Rᵥ, R_{w}, Rₓ and R_{y} are independently selected from hydrogen, alkyl, cycloalkyl, heterocyclic, aryl or heteroaryl. Alternatively, Rₓ and R_{y} together form a heterocyclic ring or 3 to 8 members, optionally substituted with unsubstituted alkyl, unsubstituted cycloalkyl, unsubstituted heterocyclic, unsubstituted aryl, unsubstituted heteroaryl, hydroxy, alkoxy, aryloxy, acyl, amino, amido, carboxy, carboxyalkyl, carboxyaryl, mercapto, sulfinyl, sulfonyl, sulfonamido, amidino, carbamoyl, guanidino or ureido, and optionally containing one to three additional heteroatoms selected from O, S or N.

The term “ureido” refers to a group of the formula —N(R_{z})—C(═O)—NRₐₐR_{bb} wherein R_{z}, Rₐₐ and R_{bb} are independently selected from hydrogen, alkyl, cycloalkyl, heterocyclic, aryl or heteroaryl. Alternatively, Rₐₐ and R_{bb} together with the nitrogen atom to which they are each bonded form a heterocyclic ring of 3 to 8 members, optionally substituted with unsubstituted alkyl, unsubstituted cycloalkyl, unsubstituted heterocyclic, unsubstituted aryl, unsubstituted heteroaryl, hydroxy, alkoxy, aryloxy, acyl, amino, amido, carboxy, carboxyalkyl, carboxyaryl, mercapto, sulfinyl, sulfonyl, sulfonamido, amidino, carbamoyl, guanidino or ureido, and optionally containing one to three additional heteroatoms selected from O, S or N.

The term “optionally substituted” is intended to expressly indicate that the specified group is unsubstituted or substituted by one or more suitable substituents, unless the optional substituents are expressly specified, in which case the term indicates that the group is unsubstituted or substituted with the specified substituents. As defined above, various groups may be unsubstituted or substituted (i.e., they are optionally substituted) unless indicated otherwise herein (e.g., by indicating that the specified group is unsubstituted).

The term “substituted” when used with the terms alkyl, cycloalkyl, heterocyclic, aryl and heteroaryl refers to an alkyl, cycloalkyl, heterocyclic, aryl or heteroaryl group having one or more of the hydrogen atoms of the group replaced by substituents independently selected from unsubstituted alkyl, unsubstituted cycloalkyl, unsubstituted heterocyclic, unsubstituted aryl, unsubstituted heteroaryl, hydroxy, alkoxy, aryloxy, acyl, amino, amido, carboxy, carboxyalkyl, carboxyaryl, halo, oxo, mercapto, sulfinyl, sulfonyl, sulfonamido, amidino, carbamoyl, guanidino, ureido and groups of the formulas —NR_{cc}C(═O)R_{dd}, —NRₑₑC(═NR_{ff})R_{gg}, —OC(═O)NRₕₕRᵢᵢ, —OC(═O)Rⱼⱼ, —OC(═O)ORₖₖ, —NRₘₘSO₂Rₙₙ, or —NRₚₚSO₂NR_{qq}Rᵣᵣ wherein R_{cc}, R_{dd}, Rₑₑ, R_{ff}, R_{gg}, Rₕₕ, Rᵢᵢ, Rⱼⱼ, Rₘₘ, Rₚₚ, R_{qq} and Rᵣᵣ are independently selected from hydrogen, unsubstituted alkyl, unsubstituted cycloalkyl, unsubstituted heterocyclic, unsubstituted aryl or unsubstituted heteroaryl; and wherein Rₖₖ and Rₙₙ are independently selected from unsubstituted alkyl, unsubstituted cycloalkyl, unsubstituted heterocyclic, unsubstituted aryl or unsubstituted heteroaryl. Alternatively, R_{gg} and Rₕₕ, Rⱼⱼ and Rₖₖ or Rₚₚ and R_{qq} together with the nitrogen atom to which they are each bonded form a heterocyclic ring of 3 to 8 members, optionally substituted with unsubstituted alkyl, unsubstituted cycloalkyl, unsubstituted heterocyclic, unsubstituted aryl, unsubstituted heteroaryl, hydroxy, alkoxy, aryloxy, acyl, amino, amido, carboxy, carboxyalkyl, carboxyaryl, mercapto, sulfinyl, sulfonyl, sulfonamido, amidino, carbamoyl, guanidino or ureido, and optionally containing one to three additional heteroatoms selected from O, S or N. In addition, the term “substituted” for aryl and heteroaryl groups includes as an option having one of the hydrogen atoms of the group replaced by cyano, nitro or trifluoromethyl.

A substitution is made provided that any atom's normal valency is not exceeded and that the substitution results in a stable compound. Generally, when a substituted form of a group is present, such substituted group may not be further substituted or, if substituted, the substituent comprises only a limited number of substituted groups, for example 1, 2, 3 or 4 such substituents.

When any variable occurs more than one time in any constituent or in any formula herein, its definition on each occurrence is independent of its definition at every other occurrence. Also, combinations of substituents and/or variables are permissible only if such combinations result in stable compounds.

A “stable compound” or “stable structure” is meant to mean a compound that is sufficiently robust to survive isolation to a useful degree of purity and formulation into an efficacious therapeutic agent.

The term “amino acid” refers to the common natural (genetically encoded) or synthetic amino acids and common derivatives thereof, known to those skilled in the art. When applied to amino acids, “standard” or “proteinogenic” refers to the genetically encoded 20 amino acids in their natural configuration. Similarly, when applied to amino acids, “unnatural” or “unusual” refers to the wide selection of non-natural, rare or synthetic amino acids such as those described by Hunt, S. in Chemistry and Biochemistry of the Amino Acids, Barrett, G. C., Ed., Chapman and Hall: New York, 1985.

The term “residue” with reference to an amino acid or amino acid derivative refers to a group of the formula:

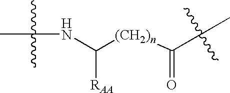

wherein R_{AA} is an amino acid side chain, and n=0, 1 or 2 in this instance.

The term “fragment” with respect to a dipeptide, tripeptide or higher order peptide derivative indicates a group that contains two, three or more, respectively, amino acid residues.

The term “amino acid side chain” refers to any side chain from a standard or unnatural amino acid, and is denoted R_{AA}. For example, the side chain of alanine is methyl, the side chain of valine is isopropyl and the side chain of tryptophan is 3-indolylmethyl.

The term “agonist” refers to a compound that duplicates at least some of the effect of the endogenous ligand of a protein, receptor, enzyme or the like.

The term “antagonist” refers to a compound that inhibits at least some of the effect of the endogenous ligand of a protein, receptor, enzyme or the like.

The term “inverse agonist” refers to a compound that decreases, at least to some degree, the baseline functional activity of a protein, receptor, enzyme or the like, such as the constitutive signaling activity of a G protein-coupled receptor or variant thereof. An inverse agonist can also be an antagonist.

The term “baseline functional activity” refers to the activity of a protein, receptor, enzyme or the like, including constitutive signaling activity, in the absence of the endogenous ligand.

The term “growth hormone secretagogue” (GHS) refers to any exogenously administered compound or agent that directly or indirectly stimulates or increases the endogenous release of growth hormone, growth hormone-releasing hormone, or somatostatin in an animal, in particular, a human. A GHS may be peptidic or non-peptidic in nature, with an agent that can be administered orally preferred. In addition, an agent that induces a pulsatile response is preferred.

The term “modulator” refers to a compound that imparts an effect on a biological or chemical process or mechanism. For example, a modulator may increase, facilitate, upregulate, activate, inhibit, decrease, block, prevent, delay, desensitize, deactivate, down regulate, or the like, a biological or chemical process or mechanism. Accordingly, a modulator can be an “agonist,” an “antagonist,” Or an “inverse agonist.” Exemplary biological processes or mechanisms affected by a modulator include, but are not limited to, receptor binding and hormone release or secretion. Exemplary chemical processes or mechanisms affected by a modulator include, but are not limited to, catalysis and hydrolysis.

The term “variant” when applied to a receptor is meant to include dimers, trimers, tetramers, pentamers and other biological complexes containing multiple components. These components can be the same or different.

The term “peptide” refers to a chemical compound comprised of two or more amino acids covalently bonded together.

The term “peptidomimetic” refers to a chemical compound designed to mimic a peptide, but which contains structural differences through the addition or replacement of one of more functional groups of the peptide in order to modulate its activity or other properties, such as solubility, metabolic stability, oral bioavailability, lipophilicity, permeability, etc. This can include replacement of the peptide bond, side chain modifications, truncations, additions of functional groups, etc. When the chemical structure is not derived from the peptide, but mimics its activity, it is often referred to as a “non-peptide peptidomimetic.”

The term “peptide bond” refers to the amide [—C(═O)—NH—] functionality with which individual amino acids are typically covalently bonded to each other in a peptide.

The term “protecting group” refers to any chemical compound that may be used to prevent a potentially reactive functional group, such as an amine, a hydroxyl or a carboxyl, on a molecule from undergoing a chemical reaction while chemical change occurs elsewhere in the molecule. A number of such protecting groups are known to those skilled in the art and examples can be found in “Protective Groups in Organic Synthesis,” Theodora W. Greene and Peter G. Wuts, editors, John Wiley & Sons, New York, 3^{rd} edition, 1999 [ISBN 0471160199]. Examples of amino protecting groups include, but are not limited to, phthalimido, trichloroacetyl, benzyloxycarbonyl, tert-butoxycarbonyl, and adamantyloxy-carbonyl. Preferred amino protecting groups are carbamate amino protecting groups, which are defined as an amino protecting group that when bound to an amino group forms a carbamate. Preferred amino carbamate protecting groups are allyloxycarbonyl (Alloc), benzyloxycarbonyl (Cbz), 9-fluorenylmethoxycarbonyl (Fmoc), tert-butoxycarbonyl (Boc) and α,α-dimethyl-3,5-dimethoxybenzyloxycarbonyl (Ddz). For a recent discussion of newer nitrogen protecting groups: Theodoridis, G. Tetrahedron 2000, 56, 2339-2358. Examples of hydroxyl protecting groups include, but are not limited to, acetyl, tert-butyldimethylsilyl (TBDMS), trityl (Trt), tert-butyl, and tetrahydropyranyl (THP). Examples of carboxyl protecting groups include, but are not limited to methyl ester, tert-butyl ester, benzyl ester, trimethylsilylethyl ester, and 2,2,2-trichloroethyl ester.

The term “solid phase chemistry” refers to the conduct of chemical reactions where one component of the reaction is covalently bonded to a polymeric material (solid support as defined below). Reaction methods for performing chemistry on solid phase have become more widely known and established outside the traditional fields of peptide and oligonucleotide chemistry.

The term “solid support,” “solid phase” or “resin” refers to a mechanically and chemically stable polymeric matrix utilized to conduct solid phase chemistry. This is denoted by “Resin,” “P-” or the following symbol:

[symbol]

Examples of appropriate polymer materials include, but are not limited to, polystyrene, polyethylene, polyethylene glycol, polyethylene glycol grafted or covalently bonded to polystyrene (also termed PEG-polystyrene, TentaGel™, Rapp, W.; Zhang, L.; Bayer, E. In Innovations and Perspectives in Solid Phase Synthesis. Peptides, Polypeptides and Oligonucleotides; Epton, R., Ed.; SPCC Ltd.: Birmingham, UK; p 205), polyacrylate (CLEAR™), polyacrylamide, polyurethane, PEGA [polyethyleneglycol poly(N,N-dimethylacrylamide) co-polymer, Meldal, M. Tetrahedron Lett. 1992, 33, 3077-3080], cellulose, etc. These materials can optionally contain additional chemical agents to form cross-linked bonds to mechanically stabilize the structure, for example polystyrene cross-linked with divinylbenezene (DVB, usually 0.1-5%, or 0.5-2%). This solid support can include as non-limiting examples aminomethyl polystyrene, hydroxymethyl polystyrene, benzhydrylamine polystyrene (BHA), methylbenzhydrylamine (MBHA) polystyrene, and other polymeric backbones containing free chemical functional groups, most typically, —NH₂ or —OH, for further derivatization or reaction. The term is also meant to include “Ultraresins” with a high proportion (“loading”) of these functional groups such as those prepared from polyethyleneimines and cross-linking molecules (Barth, M.; Rademann, J. J. Comb. Chem. 2004, 6, 340-349). At the conclusion of the synthesis, resins are typically discarded, although they have been shown to be able to be reused such as in Frechet, J. M. J.; Haque, K. E. Tetrahedron Lett. 1975, 16, 3055.

In general, the materials used as resins are insoluble polymers, but certain polymers have differential solubility depending on solvent and can also be employed for solid phase chemistry. For example, polyethylene glycol can be utilized in this manner since it is soluble in many organic solvents in which chemical reactions can be conducted, but it is insoluble in others, such as diethyl ether. Hence, reactions can be conducted homogeneously in solution, then the product on the polymer precipitated through the addition of diethyl ether and processed as a solid. This has been termed “liquid-phase” chemistry.

The term “linker” when used in reference to solid phase chemistry refers to a chemical group that is bonded covalently to a solid support and is attached between the support and the substrate typically in order to permit the release (cleavage) of the substrate from the solid support. However, it can also be used to impart stability to the bond to the solid support or merely as a spacer element. Many solid supports are available commercially with linkers already attached.

Abbreviations used for amino acids and designation of peptides follow the rules of the IUPAC-IUB Commission of Biochemical Nomenclature in J. Biol. Chem. 1972, 247, 977-983. This document has been updated: Biochem. J, 1984, 219, 345-373; Eur. J. Biochem., 1984, 138, 9-37; 1985, 152, 1; Int. J. Pept. Prot. Res., 1984, 24, following p 84; J. Biol. Chem., 1985, 260, 14-42; Pure Appl. Chem., 1984, 56, 595-624; Amino Acids and Peptides, 1985, 16, 387-410; and in Biochemical Nomenclature and Related Documents, 2nd edition, Portland Press, 1992, pp 39-67. Extensions to the rules were published in the JCBN/NC-IUB Newsletter 1985, 1986, 1989; see Biochemical Nomenclature and Related Documents, 2nd edition, Portland Press, 1992, pp 68-69.

The term “effective amount” or “effective” is intended to designate a dose that causes a relief of symptoms of a disease or disorder as noted through clinical testing and evaluation, patient observation, and the like, and/or a dose that causes a detectable change in biological or chemical activity as detected by one skilled in the art for the relevant mechanism or process. As is generally understood in the art, the dosage will vary depending on the administration routes, symptoms and body weight of the patient but also depending upon the compound being administered.

Administration of two or more compounds “in combination” means that the two compounds are administered closely enough in time that the presence of one alters the biological effects of the other. The two compounds can be administered simultaneously (concurrently) or sequentially. Simultaneous administration can be carried out by mixing the compounds prior to administration, or by administering the compounds at the same point in time but at different anatomic sites or using different routes of administration. The phrases “concurrent administration”, “administration in combination”, “simultaneous administration” or “administered simultaneously” as used herein, means that the compounds are administered at the same point in time or immediately following one another. In the latter case, the two compounds are administered at times sufficiently close that the results observed are indistinguishable from those achieved when the compounds are administered at the same point in time.

The term “pharmaceutically active metabolite” is intended to mean a pharmacologically active product produced through metabolism in the body of a specified compound.

The term “solvate” is intended to mean a pharmaceutically acceptable solvate form of a specified compound that retains the biological effectiveness of such compound. Examples of solvates, without limitation, include compounds of the invention in combination with water, isopropanol, ethanol, methanol, DMSO, ethyl acetate, acetic acid, or ethanolamine.

### 1. Compounds

Novel macrocyclic compounds of the present invention include those of formula I:

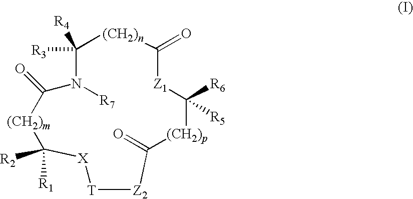

or an optical isomer, enantiomer, diastereomer, racemate or stereochemical mixture thereof, wherein the substituents are described above.

In some embodiments, R₃ is H; R₄ is —CR₄₃ₐR_{43b}(OR₄₄) where R₄₃ₐ and R_{43b} are each independently hydrogen, lower alkyl or substituted lower alkyl and R₄₄ is hydrogen, alkyl, substituted alkyl, aryl, substituted aryl, or acyl; and R₇ is hydrogen or lower alkyl.

In other embodiments, the compound can have any of the following structures:

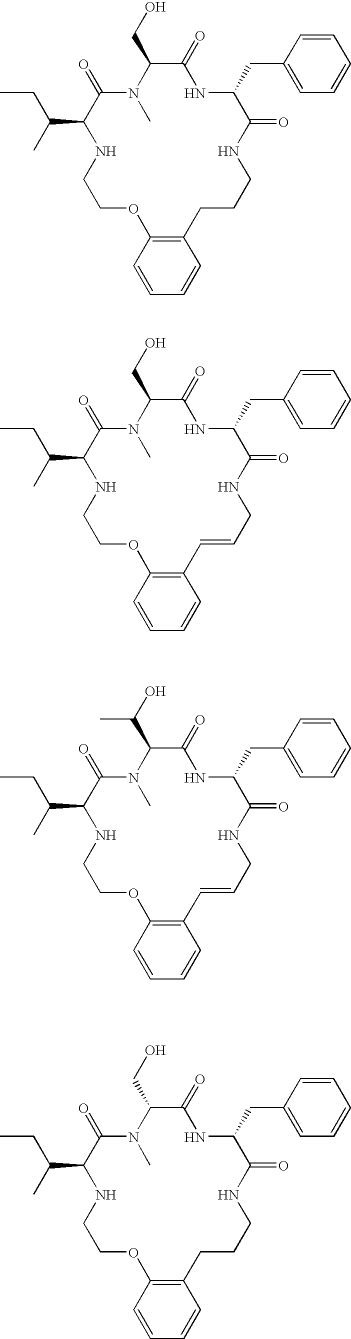

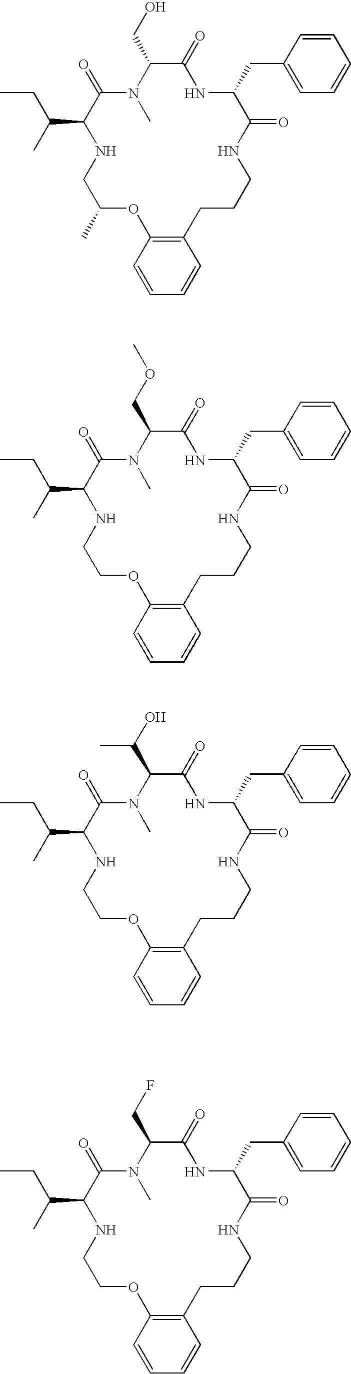

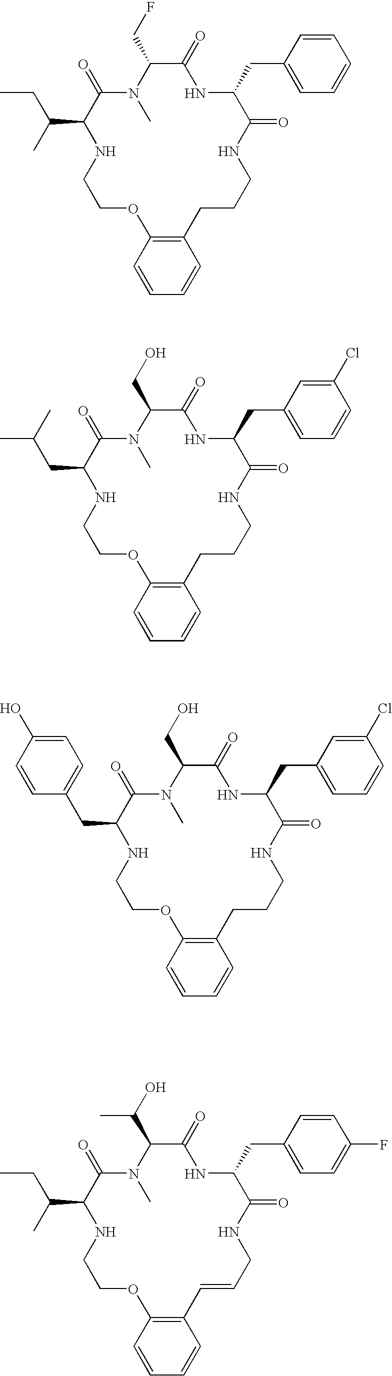

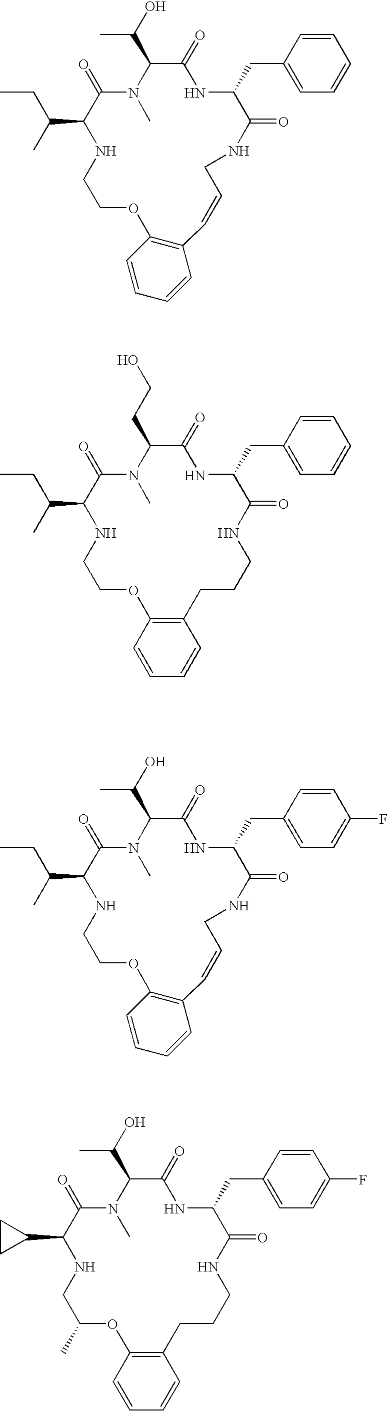

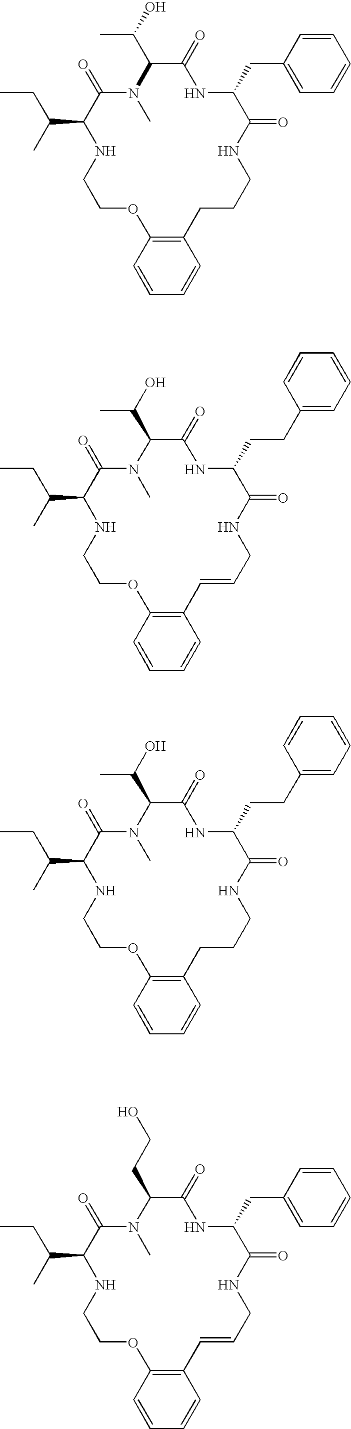

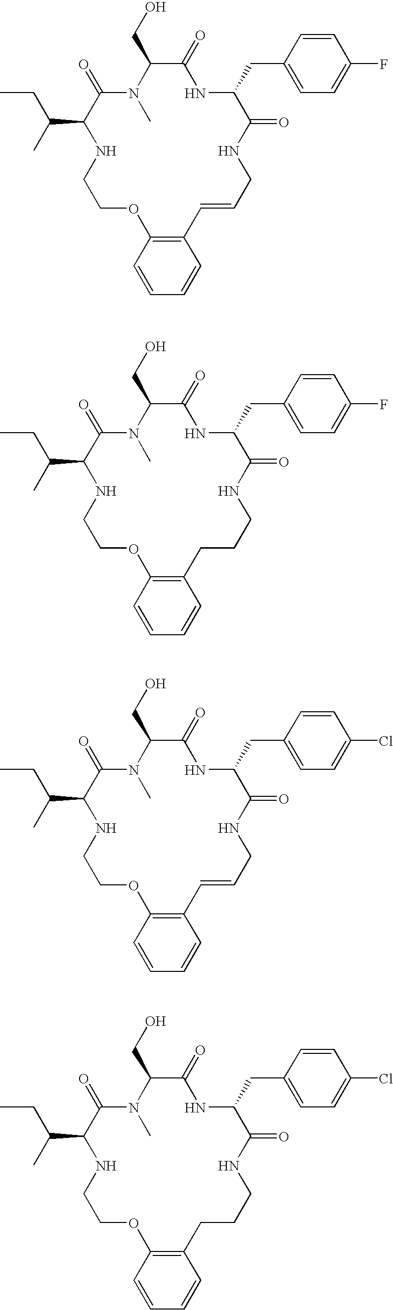

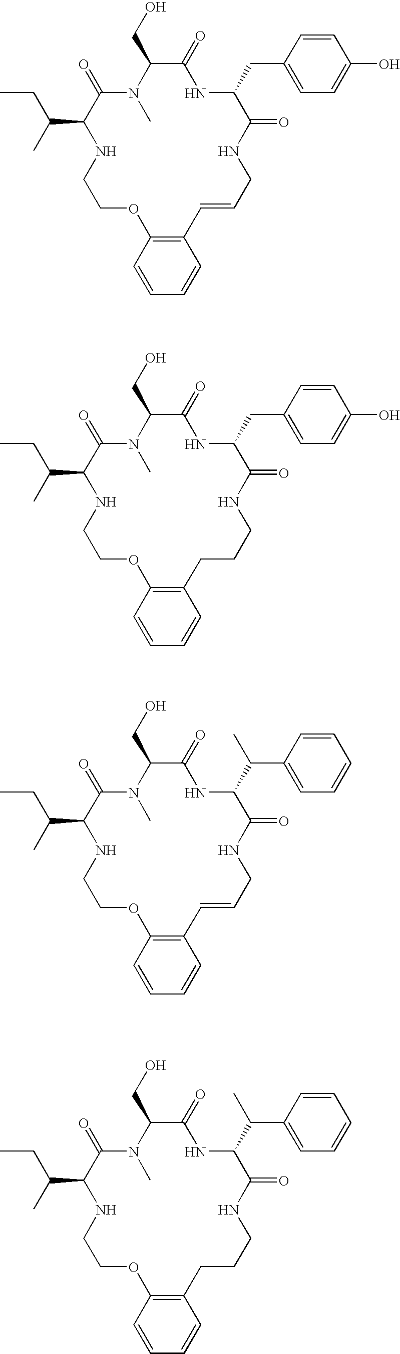

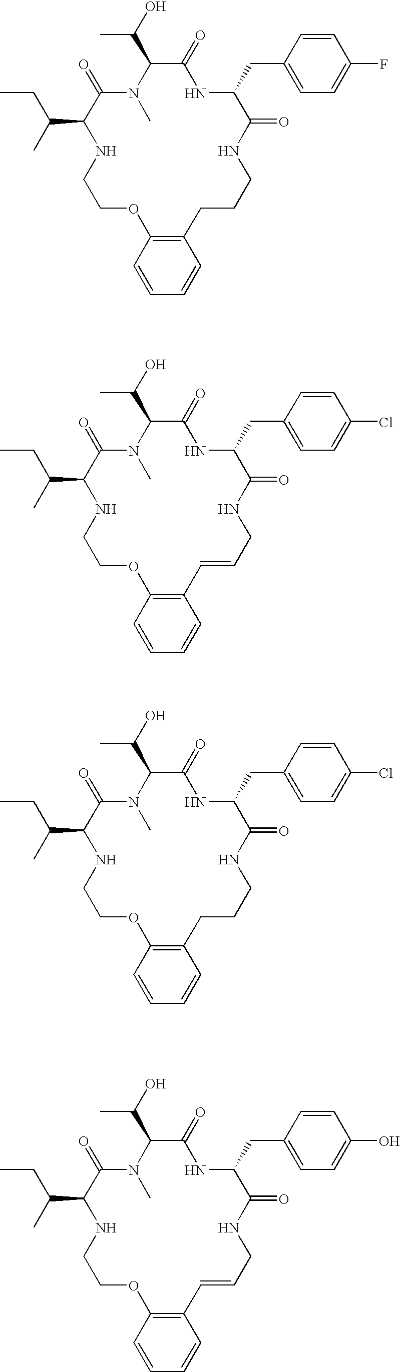

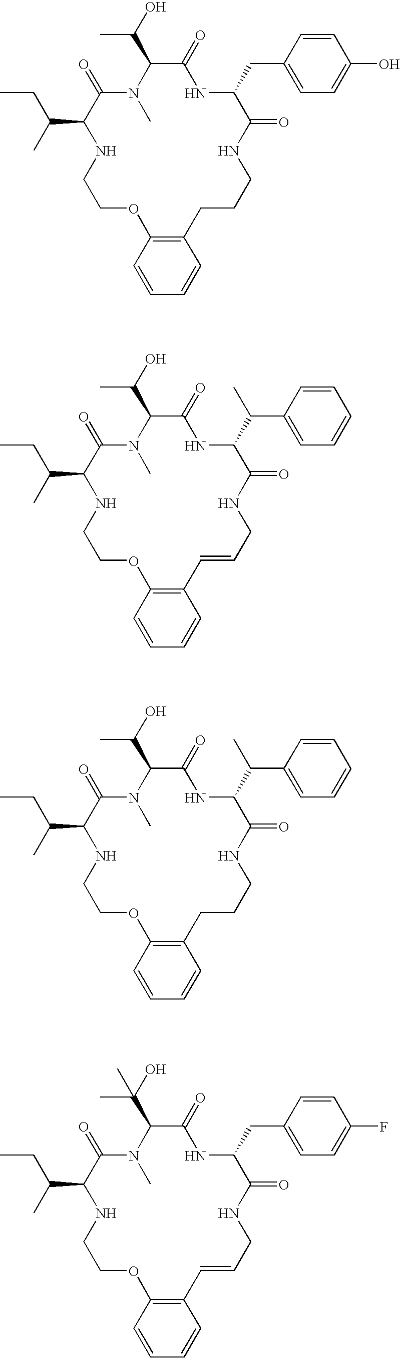

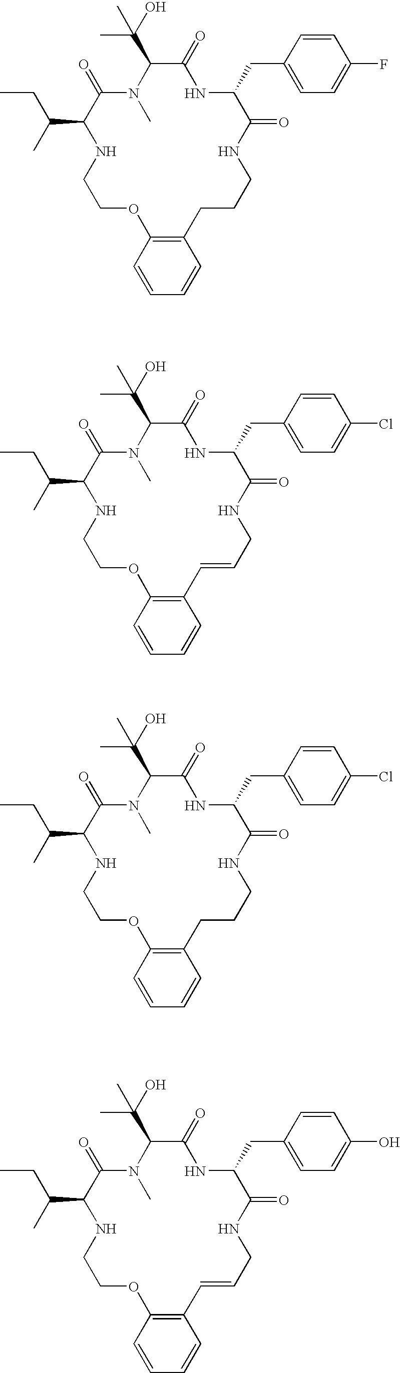

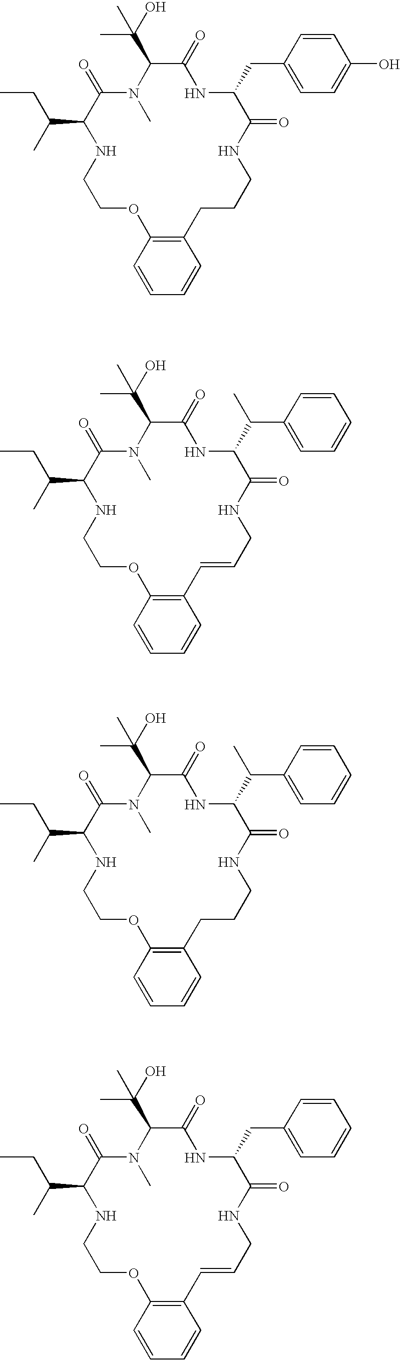

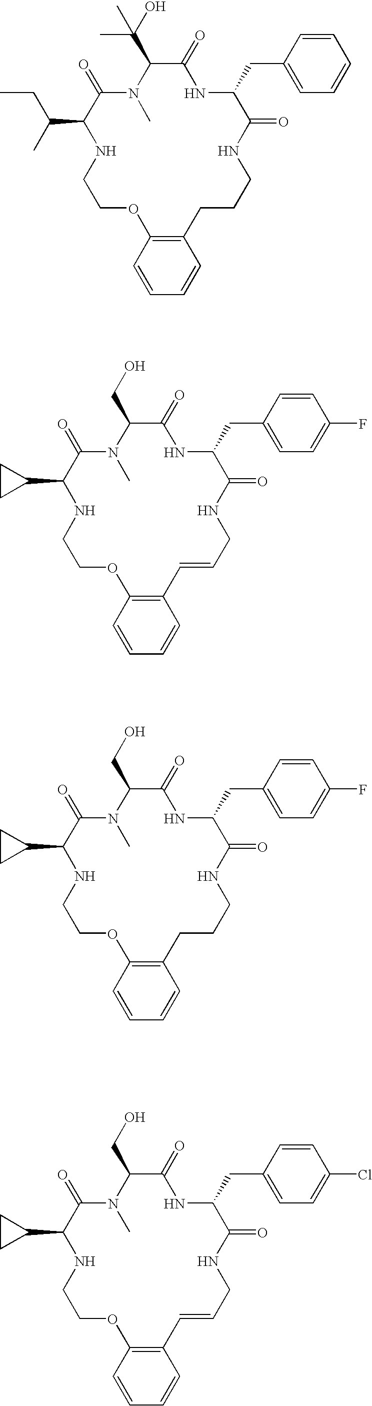

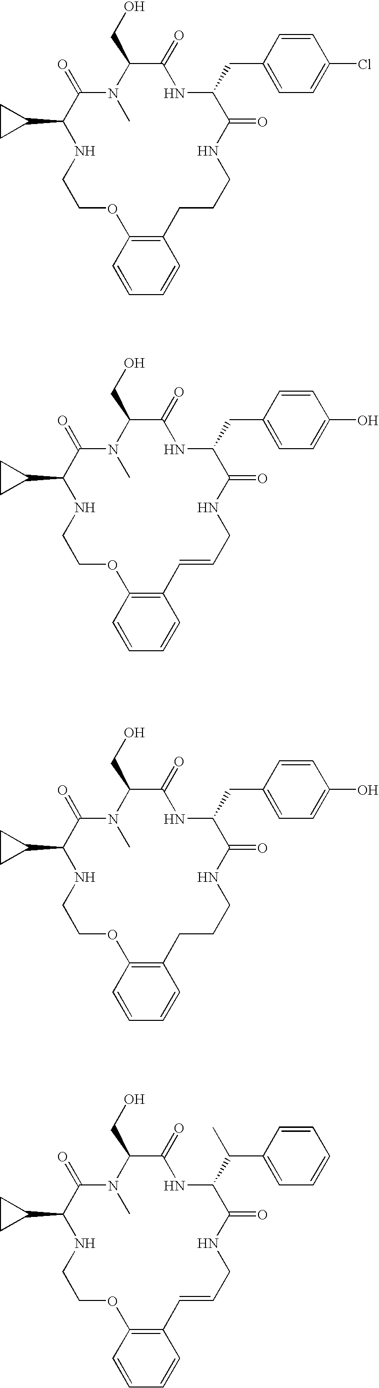

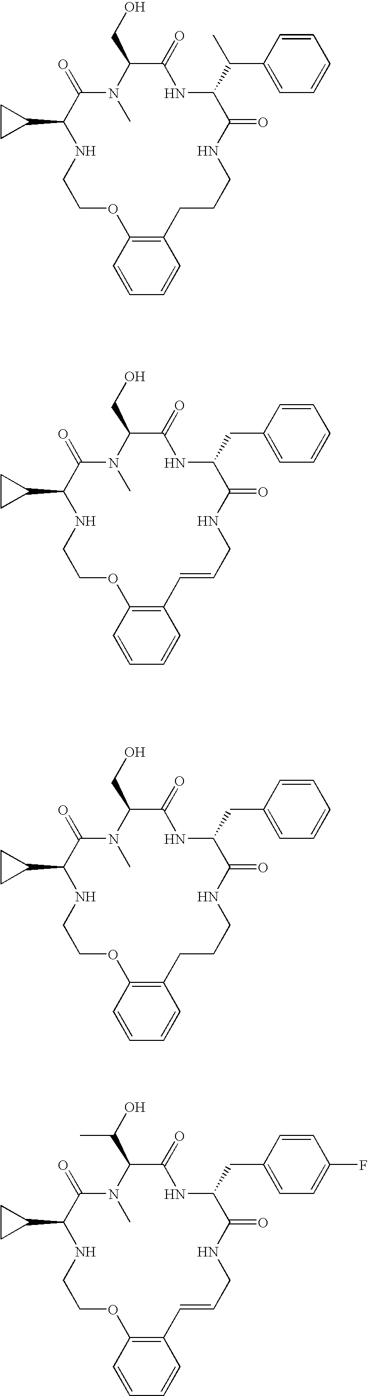

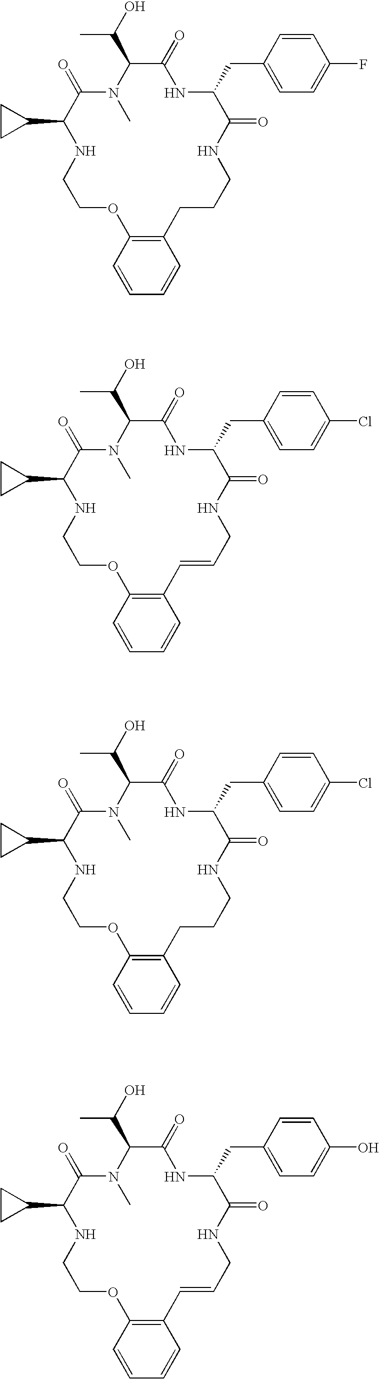

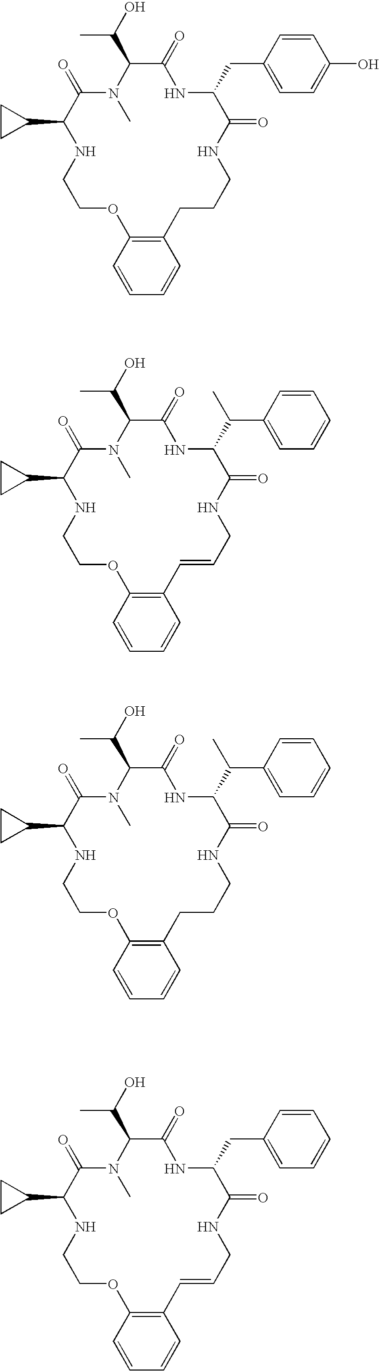

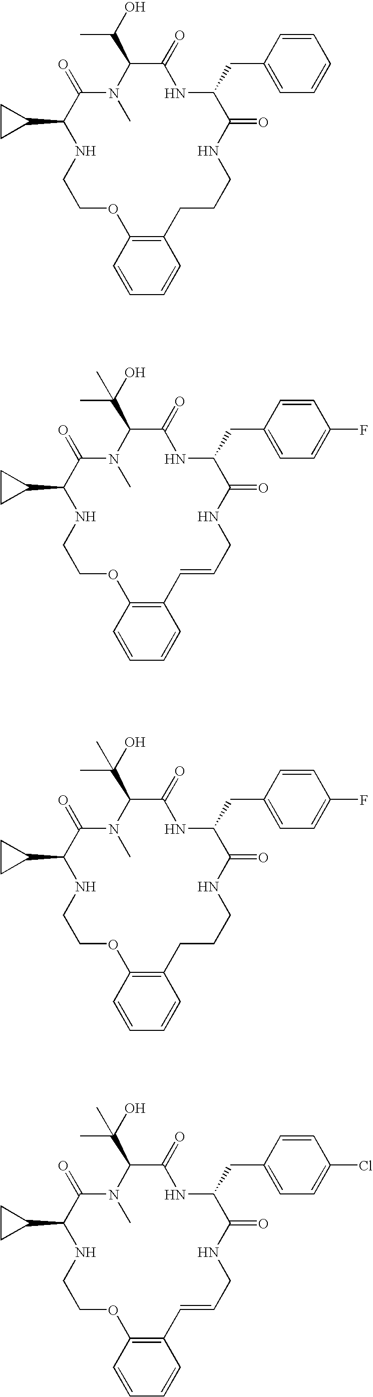

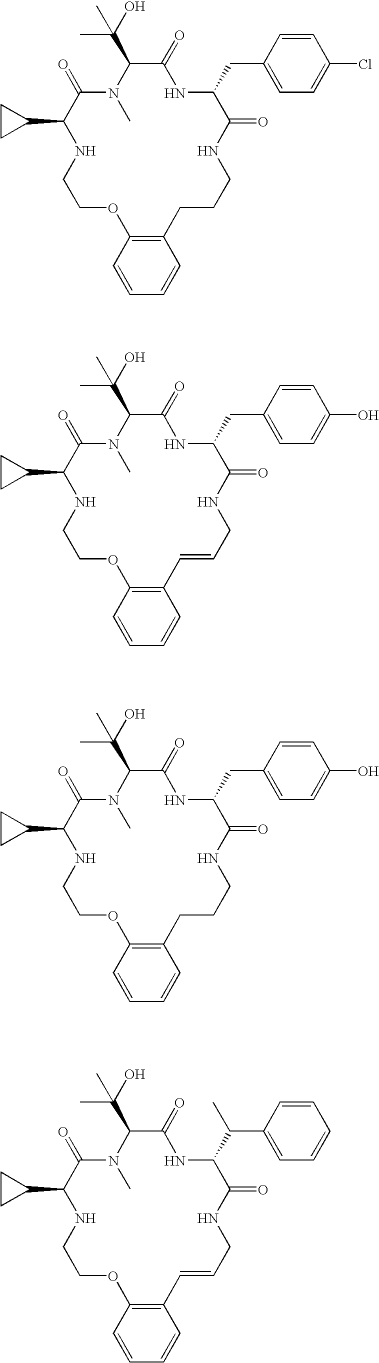

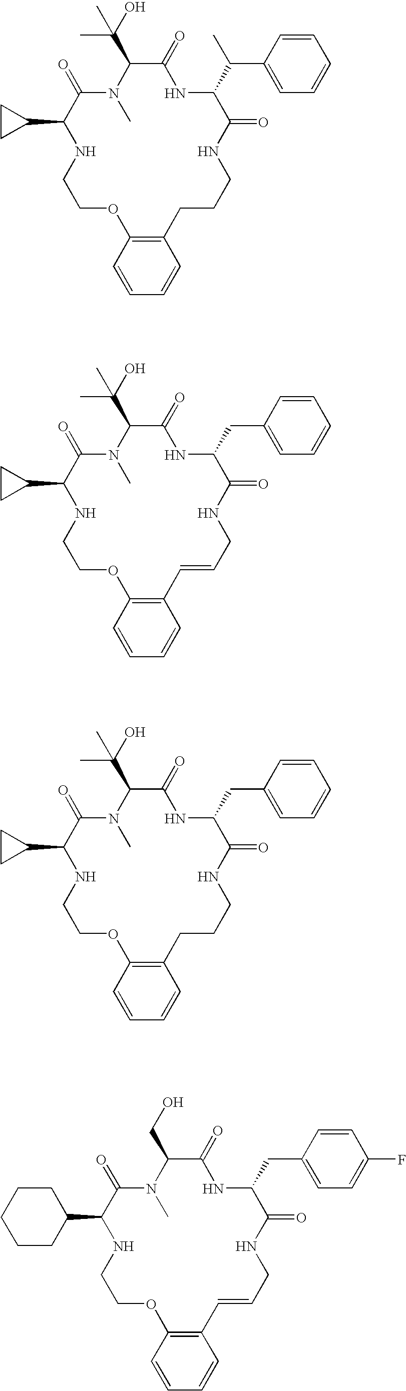

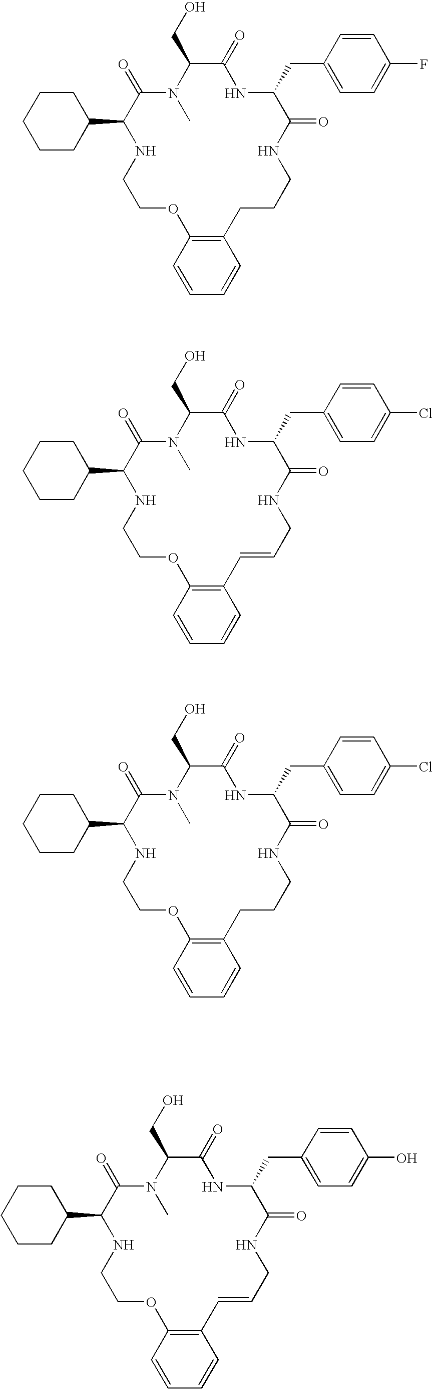

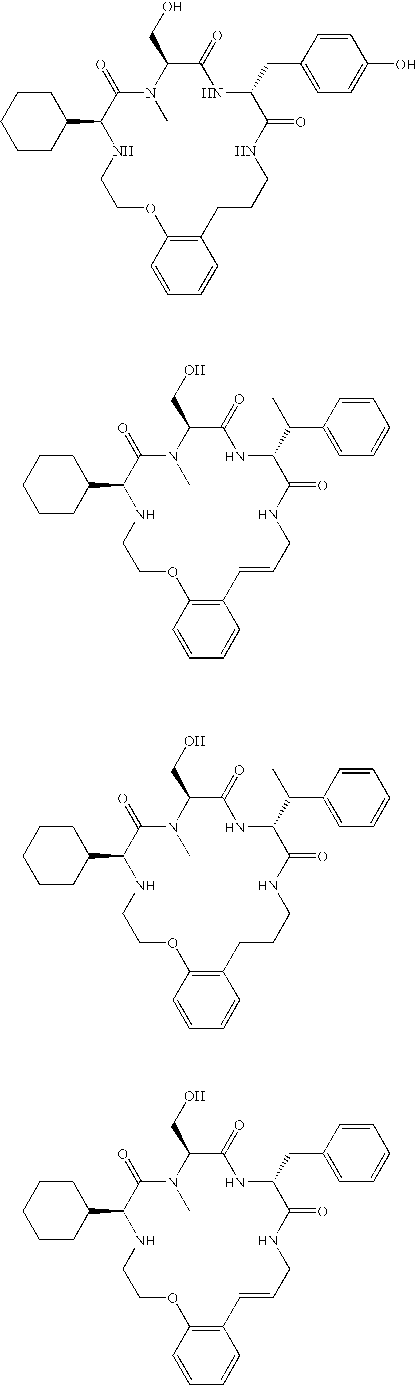

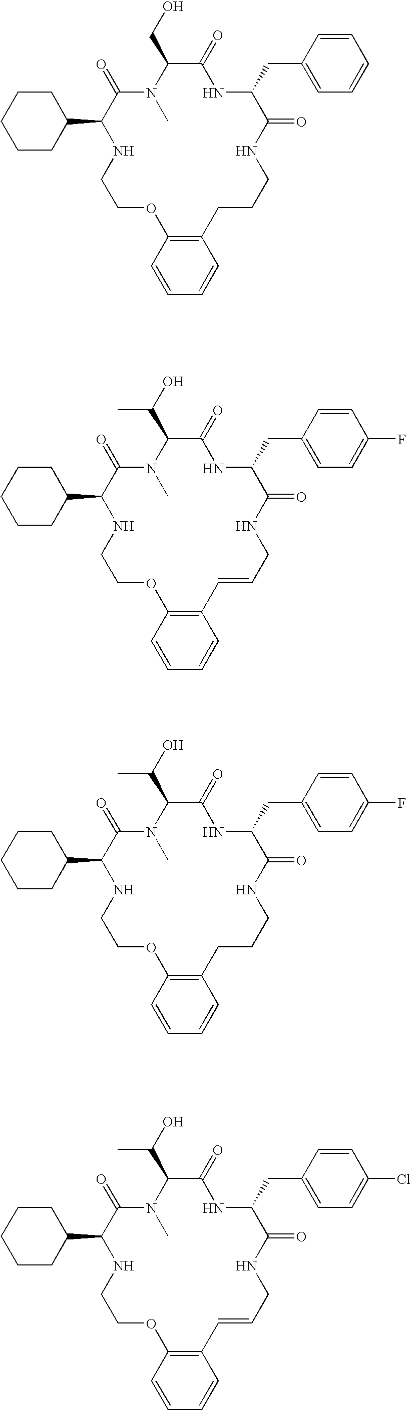

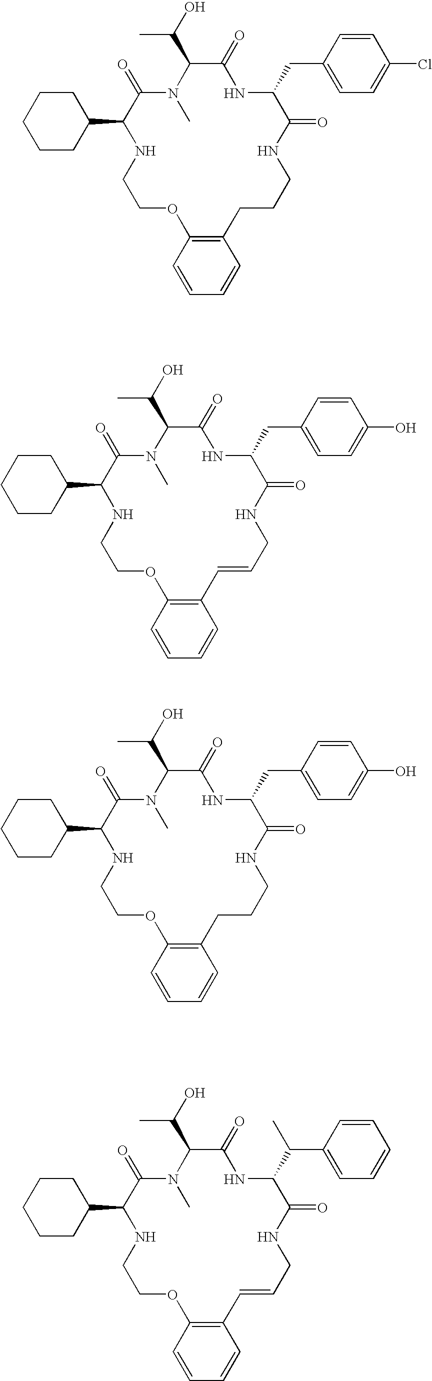

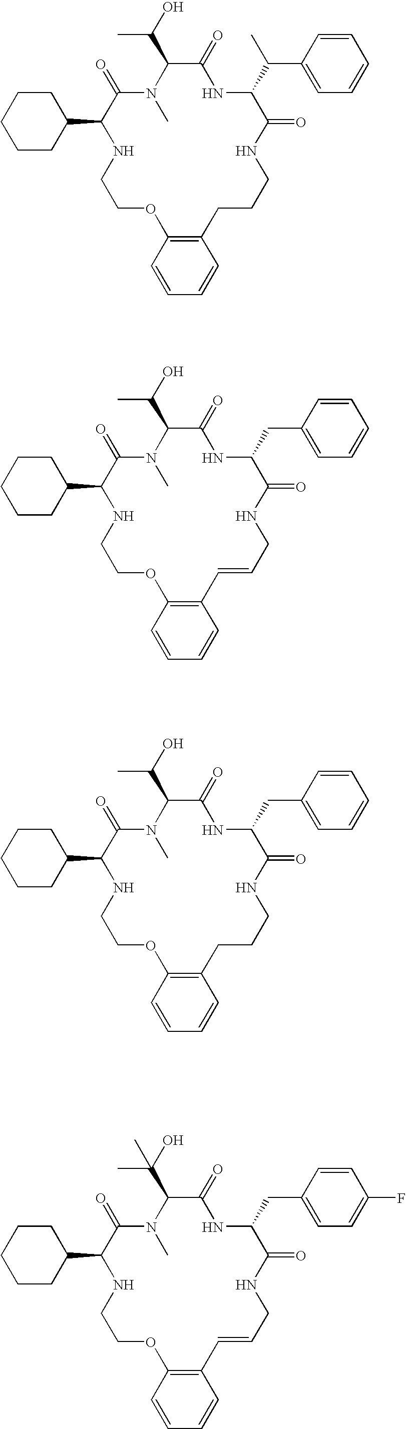

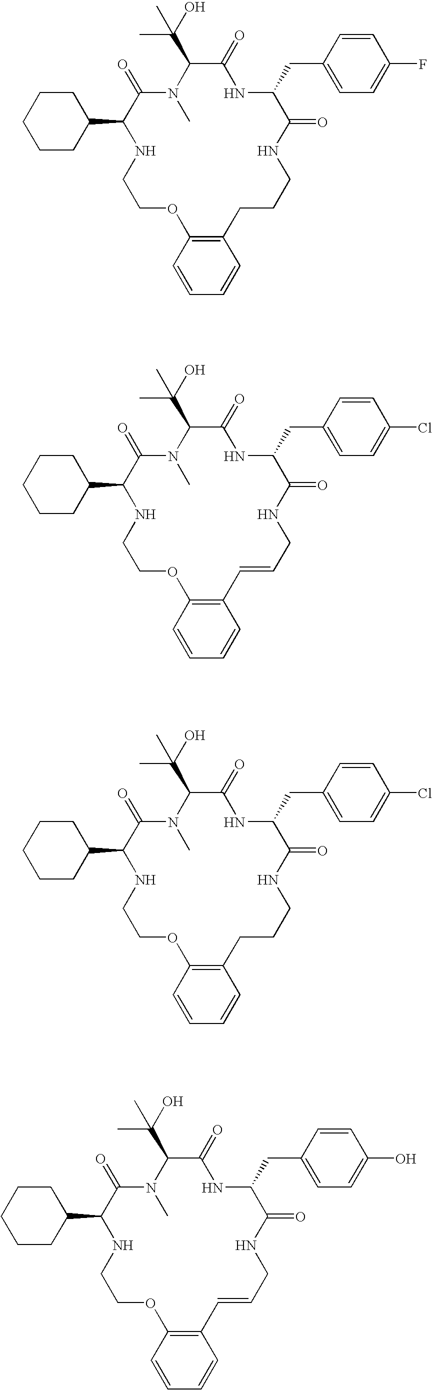

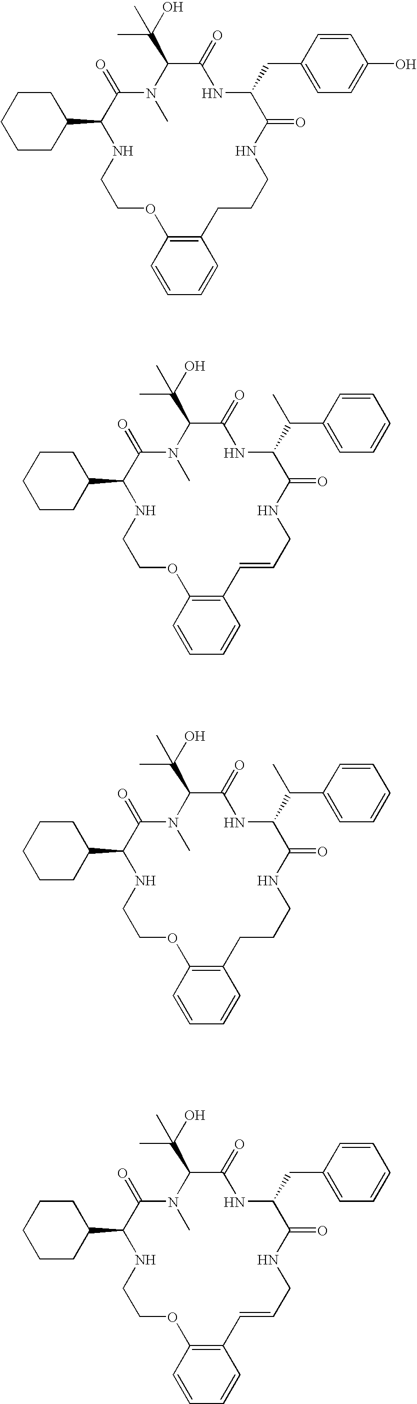

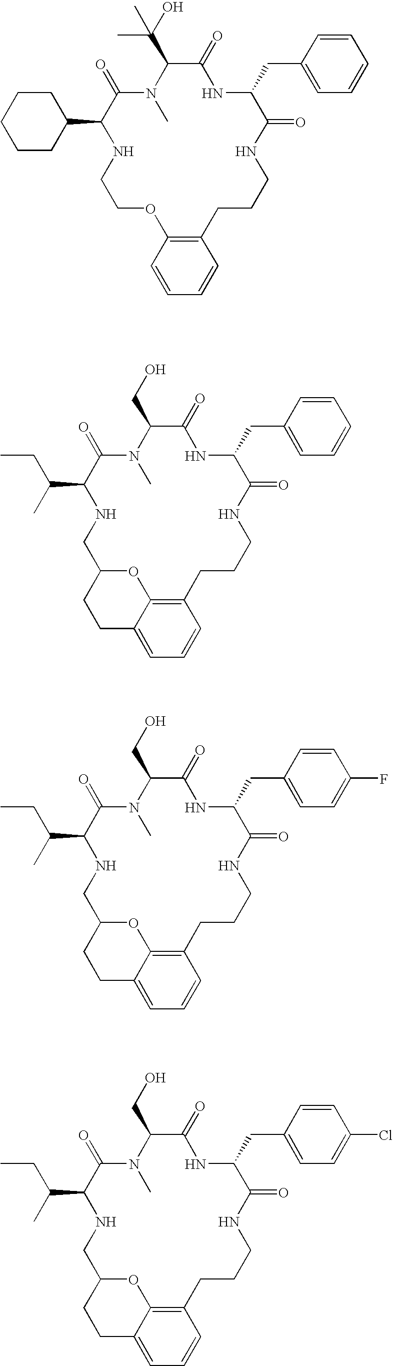

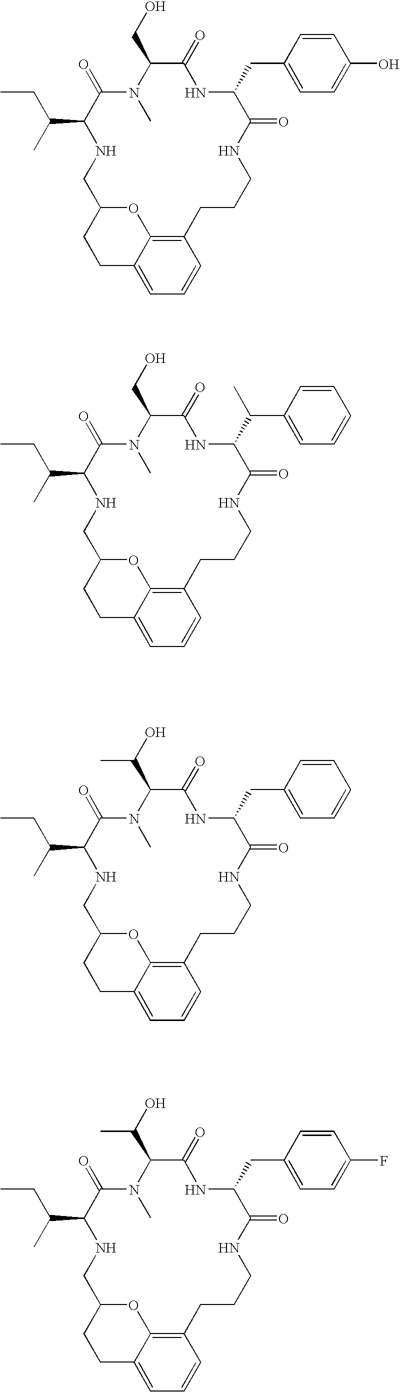

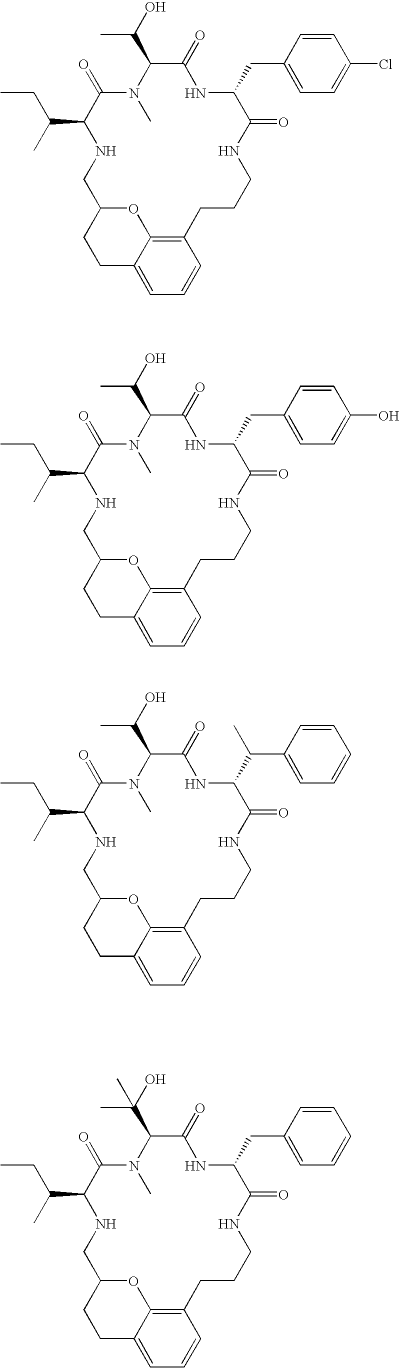

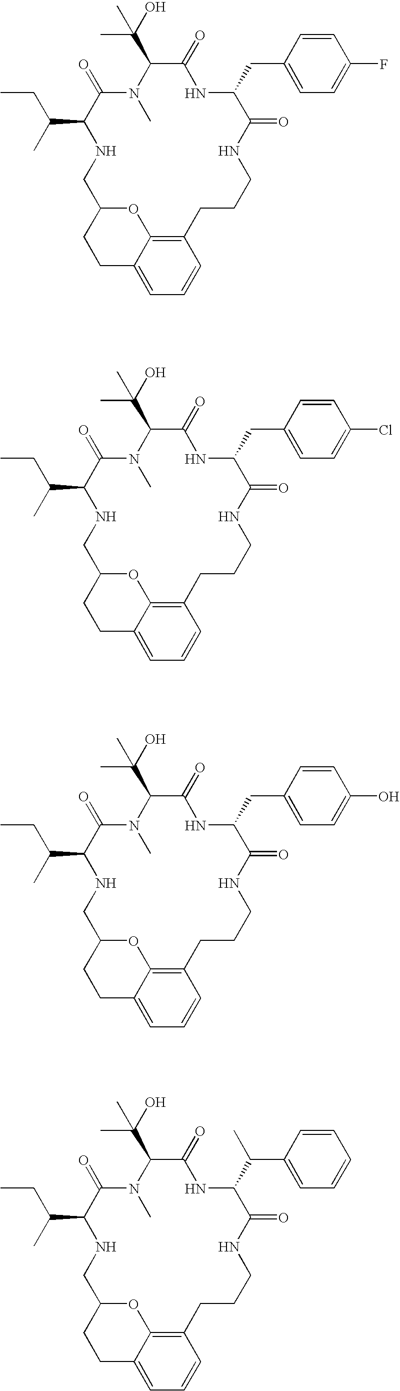

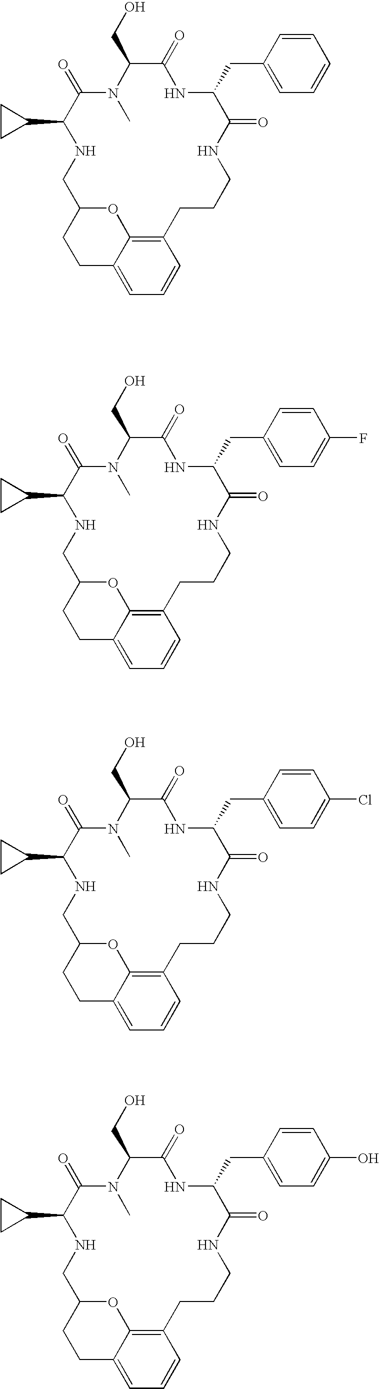

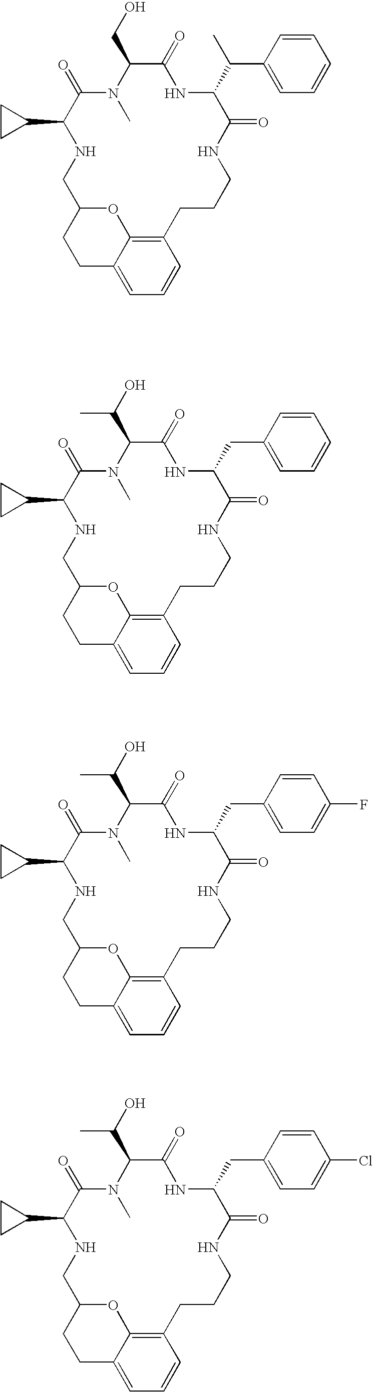

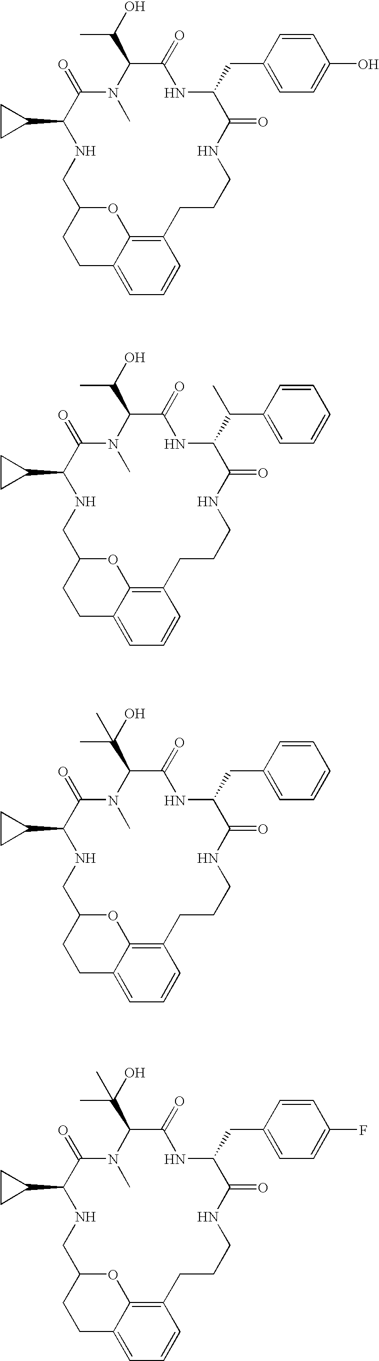

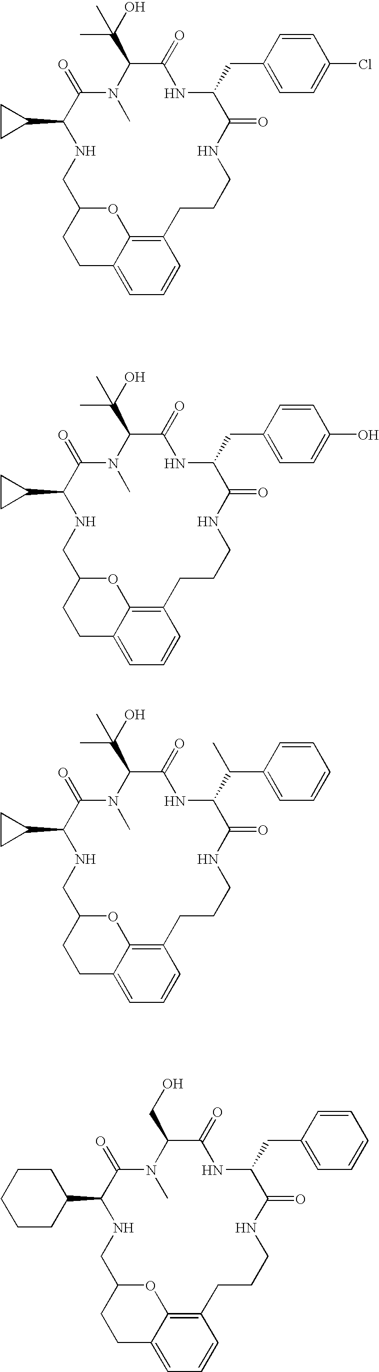

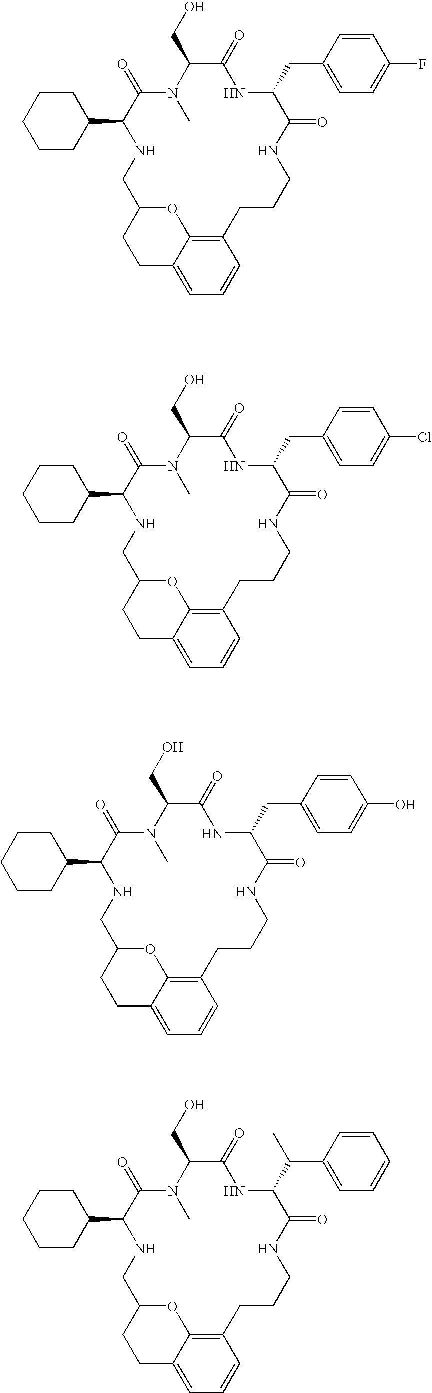

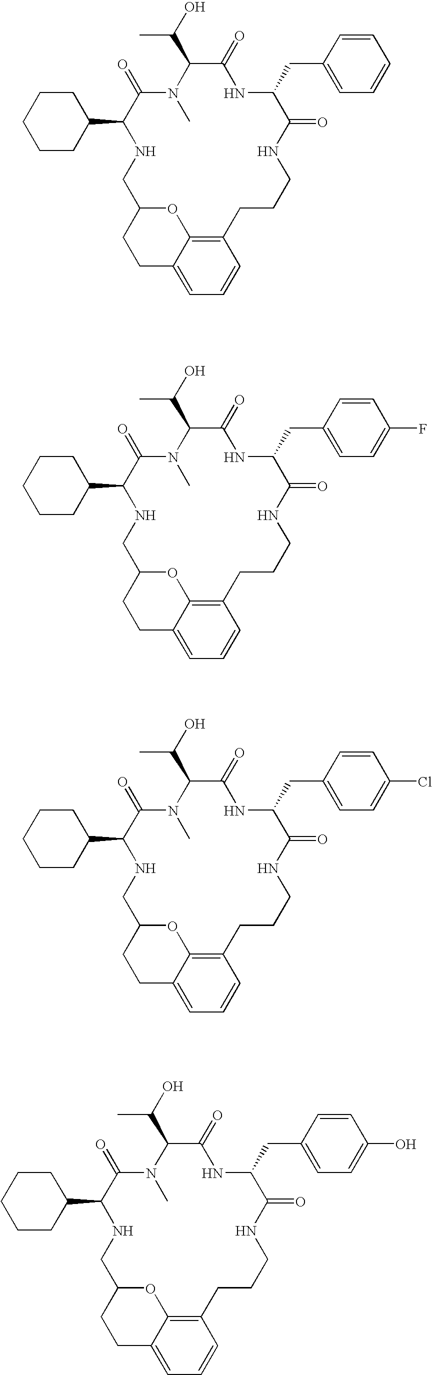

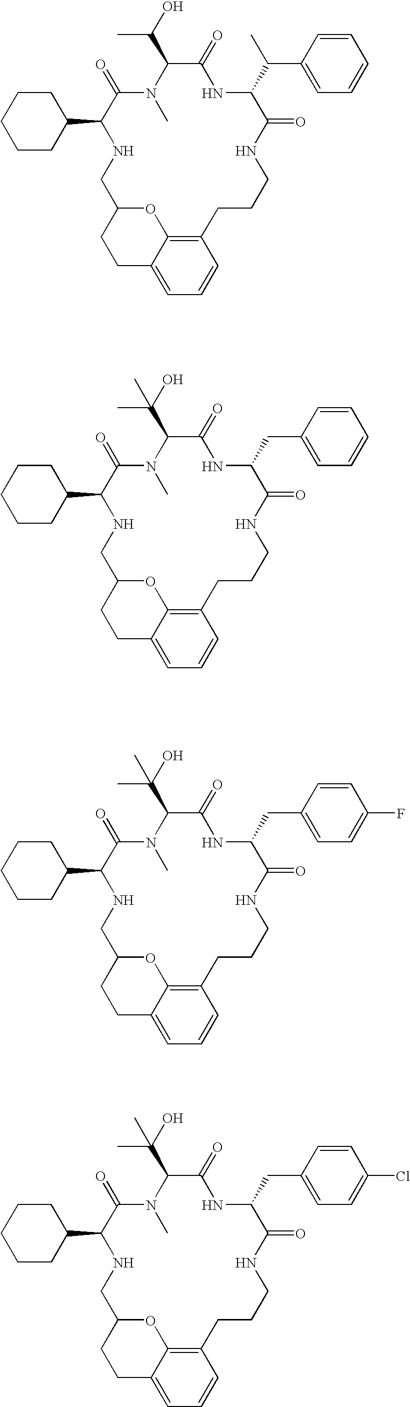

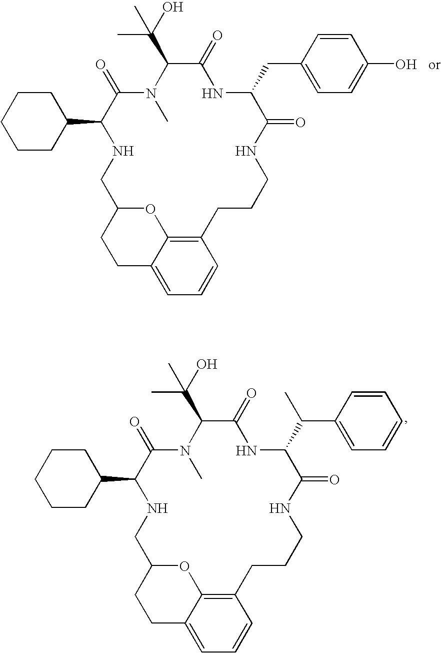

an optical isomer, enantiomer, diastereomer, racemate or stereochemical mixture thereof.

The present invention includes isolated compounds. An isolated compound refers to a compound that, in some embodiments, comprises at least 10%, at least 25%, at least 50% or at least 70% of the compounds of a mixture. In some embodiments, the compound, pharmaceutically acceptable salt thereof or pharmaceutical composition containing the compound exhibits a statistically significant binding and/or antagonist activity when tested in biological assays at the human ghrelin receptor.

In the case of compounds, salts, or solvates that are solids, it is understood by those skilled in the art that the inventive compounds, salts, and solvates may exist in different crystal or polymorphic forms, all of which are intended to be within the scope of the present invention and specified formulas.

The compounds of formula I herein disclosed have asymmetric centers. The inventive compounds may exist as single stereoisomers, racemates, and/or mixtures of enantiomers and/or diastereomers. All such single stereoisomers, racemates, and mixtures thereof are intended to be within the scope of the present invention. However, the inventive compounds are used in optically pure form. The terms “S” and “R” configuration as used herein are as defined by the IUPAC 1974 Recommendations for Section E, Fundamentals of Stereochemistry (Pure Appl. Chem. 1976, 45, 13-30).

Unless otherwise depicted to be a specific orientation, the present invention accounts for all stereoisomeric forms. The compounds may be prepared as a single stereoisomer or a mixture of stereoisomers. The non-racemic forms may be obtained by either synthesis or resolution. The compounds may, for example, be resolved into the component enantiomers by standard techniques, for example formation of diastereomeric pairs via salt formation. The compounds also may be resolved by covalently bonding to a chiral moiety. The diastereomers can then be resolved by chromatographic separation and/or crystallographic separation. In the case of a chiral auxiliary moiety, it can then be removed. As an alternative, the compounds can be resolved through the use of chiral chromatography. Enzymatic methods of resolution could also be used in certain cases.

As generally understood by those skilled in the art, an “optically pure” compound is one that contains only a single enantiomer. As used herein, the term “optically active” is intended to mean a compound comprising at least a sufficient excess of one enantiomer over the other such that the mixture rotates plane polarized light. The enantiomeric excess (e.e.) indicates the excess of one enantiomer over the other. Optically active compounds have the ability to rotate the plane of polarized light. In describing an optically active compound, the prefixes D and L or R and S are used to denote the absolute configuration of the molecule about its chiral center(s). The prefixes “d” and “l” or (+) and (−) are used to denote the optical rotation of the compound (i.e., the direction in which a plane of polarized light is rotated by the optically active compound). The “l” or (−) prefix indicates that the compound is levorotatory (i.e., rotates the plane of polarized light to the left or counterclockwise) while the “d” or (+) prefix means that the compound is dextrarotatory (i.e., rotates the plane of polarized light to the right or clockwise). The sign of optical rotation, (−) and (+), is not related to the absolute configuration of the molecule, R and S.

A compound of the invention having the desired pharmacological properties will be optically active and is comprised of at least 90% (80% e.e.), at least 95% (90% e.e.), at least 97.5% (95% e.e.) or at least 99% (98% e.e.) of a single isomer.

Likewise, many geometric isomers of double bonds and the like can also be present in the compounds disclosed herein, and all such stable isomers are included within the present invention unless otherwise specified. Also included in the invention are tautomers and rotamers of formula I.

The use of the following symbols at the right refers to substitution of one or more hydrogen atoms of the indicated ring

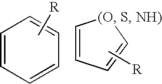

with the defined substituent R.

The use of the following symbol indicates a single bond or an optional double bond:

[symbol]

Embodiments of the present invention further provide intermediate compounds formed through the synthetic methods described herein to provide the compounds of formula I. The intermediate may possess utility as a therapeutic agent and/or reagent for further synthesis methods and reactions.

### 2. Synthetic Methods

The compounds of formula I can be synthesized using traditional solution synthesis techniques or solid phase chemistry methods. In either, the construction involves four phases: first, synthesis of the building blocks comprising recognition elements for the biological target receptor, plus one tether moiety, primarily for control and definition of conformation. These building blocks are assembled together, typically in a sequential fashion, in a second phase employing standard chemical transformations. The precursors from the assembly are then cyclized in the third stage to provide the macrocyclic structures. Finally, the post-cyclization processing fourth stage involving removal of protecting groups and optional purification provides the desired final compounds. Synthetic methods for this general type of macrocyclic structure are described in Intl. Pat. Appls. WO 01/25257, WO 2004/111077, WO 2005/012331, WO 2005/012332, WO 2006/009645 and WO 2006/009674 including purification procedures described in WO 2004/111077 and WO 2005/012331.

In some embodiments of the present invention, the macrocyclic compounds of formula I may be synthesized using solid phase chemistry on a soluble or insoluble polymer matrix as previously defined. For solid phase chemistry, a preliminary stage involving the attachment of the first building block, also termed “loading,” to the resin must be performed. The resin utilized for the present invention preferentially has attached to it a linker moiety, L. These linkers are attached to an appropriate free chemical functionality, usually an alcohol or amine, although others are also possible, on the base resin through standard reaction methods known in the art, such as any of the large number of reaction conditions developed for the formation of ester or amide bonds. Some linker moieties for the present invention are designed to allow for simultaneous cleavage from the resin with formation of the macrocycle in a process generally termed “cyclization-release.” (van Maarseveen, J. H. Solid phase synthesis of heterocycles by cyclization/cleavage methodologies. Comb. Chem. High Throughput Screen. 1998, 1, 185-214; Ian W. James, Linkers for solid phase organic synthesis. Tetrahedron 1999, 55, 4855-4946; Eggenweiler, H.-M. Linkers for solid-phase synthesis of small molecules: coupling and cleavage techniques. Drug Discovery Today 1998, 3, 552-560; Backes, B. J.; Ellman, J. A. Solid support linker strategies. Curr. Opin. Chem. Biol. 1997, 1, 86-93. Of particular utility in this regard for compounds of the invention is the 3-thiopropionic acid linker. Hojo, H.; Aimoto, S. Bull. Chem. Soc. Jpn. 1991, 64, 111-117; Zhang, L.; Tam, J. J. Am. Chem. Soc. 1999, 121, 3311-3320).

Such a process provides material of higher purity as only cyclic products are released from the solid support and minimal contamination with the linear precursor occurs as would happen in solution phase. After sequential assembly of all the building blocks and tether into the linear precursor using known or standard reaction chemistry for the formation of ester or amide bonds, base-mediated intramolecular attack on the carbonyl attached to this linker by an appropriate nucleophilic functionality that is part of the tether building block results in formation of the amide or ester bond that completes the cyclic structure as shown (Scheme 1). An analogous methodology adapted to solution phase can also be applied as would likely be preferable for larger scale applications.

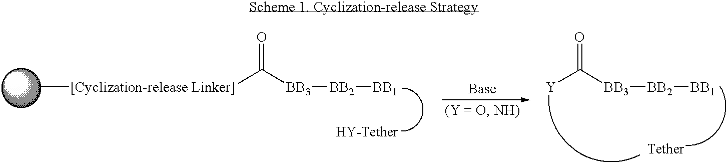

Although this description accurately represents the pathway for one of the methods of the present invention, the thioester strategy, another method of the present invention, that of ring-closing metathesis (RCM), proceeds through a modified route where the tether component is actually assembled during the cyclization step. However, in the RCM methodology as well, assembly of the building blocks proceeds sequentially, followed by cyclization (and release from the resin if solid phase). An additional post-cyclization processing step is required to remove particular byproducts of the RCM reaction, but the remaining subsequent processing is done in the same manner as for the thioester or analogous base-mediated cyclization strategy.

Moreover, it will be understood that steps including the methods provided herein may be performed independently or at least two steps may be combined. Additionally, steps including the methods provided herein, when performed independently or combined, may be performed at the same temperature or at different temperatures without departing from the teachings of the present invention.

Accordingly, the present invention provides methods of manufacturing the compounds of the present invention comprising (a) assembling building block structures, (b) chemically transforming the building block structures, (c) cyclizing the building block structures including a tether component, (d) removing protecting groups from the building block structures, and (e) optionally purifying the product obtained from step (d). In some embodiments, assembly of the building block structures may be sequential. In further embodiments, the synthesis methods are carried out using traditional solution synthesis techniques or solid phase chemistry techniques.

### A. Amino Acids

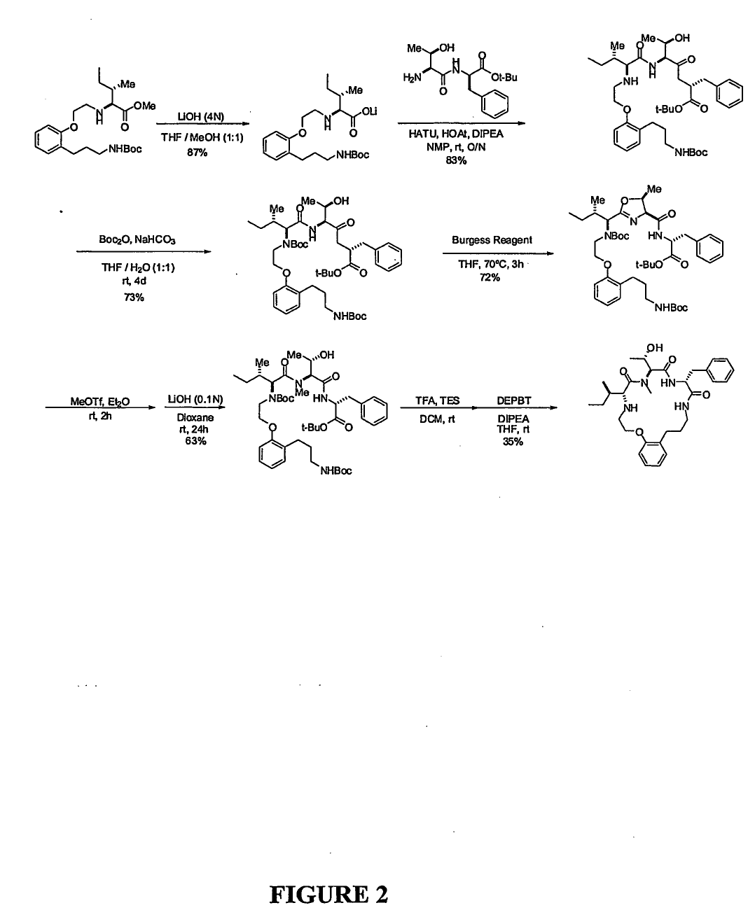

Amino acids, Boc- and Fmoc-protected amino acids and side chain protected derivatives, including those of N-methyl and unnatural amino acids, were obtained from commercial suppliers [for example Advanced ChemTech (Louisville, Ky., USA), Astatech (Princeton, N.J., USA), Bachem (Bubendorf, Switzerland), ChemInpex (Wood Dale, Ill., USA), Novabiochem (subsidiary of Merck KGaA, Darmstadt, Germany), PepTech (Burlington, Mass., USA), Synthetech (Albany, Oreg., USA)] or synthesized through standard methodologies known to those in the art. Ddz-amino acids were either obtained commercially from Orpegen (Heidelberg, Germany) or Advanced ChemTech (Louisville, Ky., USA) or synthesized using standard methods utilizing Ddz-OPh or Ddz-N₃. (Birr, C.; Lochinger, W.; Stahnke, G.; Lang, P. The α,α-dimethyl-3,5-dimethoxybenzyloxycarbonyl (Ddz) residue, an N-protecting group labile toward weak acids and irradiation. Justus Liebigs Ann. Chem. 1972, 763, 162-172). Bts-amino acids were synthesized by known methods. (Vedejs, E.; Lin, S.; Klapara, A.; Wang, J. “Heteroarene-2-sulfonyl Chlorides (BtsCl, ThsCl): Reagents for Nitrogen Protection and >99% Racemization-Free Phenylglycine Activation with SOCl₂.” J. Am. Chem. Soc. 1996, 118, 9796-9797; also WO 01/25257, WO 2004/111077) N-Alkyl amino acids, in particular, N-methyl amino acids are commercially available from multiple vendors (Bachem, Novabiochem, Advanced ChemTech, ChemImpex). In addition, N-alkyl amino acid derivatives were accessed via literature methods. (Hansen, D. W., Jr.; Pilipauskas, D. J. Org. Chem. 1985, 50, 945-950). allo-Threonine and β-hydroxyvaline can be synthesized by known procedures (Shao, H.; Goodman, M. An Enantiomeric Synthesis of allo-Threonines and β-Hydroxyvalines. J. Org. Chem. 1996, 61, 2582; Blaskovich, M. A.; Evindar, G.; Rose, N. G. W.; Wilkinson, S.; Luo, Y.; Lajoie, G. A. Stereoselective Synthesis of Threo and Erythro β-Hydroxy and β-Disubstituted-β-Hydroxy α-Amino Acids. J. Org. Chem. 1998, 63, 3631; Dettwiler; J. E. Lubell, W. D. Serine as Chiral Educt for the Practical Synthesis of Enantiopure N-Protected β-Hydroxyvaline. J. Org. Chem. 2003, 68, 177-179). Chiral isomers of β-methylphenylalanines and β-methyltyrosines can be accessed using literature methods. (Dharanipragada, R.; Van Hulle, K.; Bannister, A.; Bear, S.; Kennedy, L.; Hruby, V. J. Asymmetric Synthesis of Unusual Amino Acids: An Efficient Synthesis of Optically Pure Isomers of β-Methylphenylalanine. Tetrahedron 1992, 48, 4733-4748; Nicolas, E.; Russell, K. C.; Knollenberg, J.; Hruby, V. J. Efficient Method for the Total Asymmetric Synthesis of the Isomers of β-Methyltyrosine. J. Org. Chem. 1993, 59, 7565-7571). Incorporation of the allo-isomer of L-threonine (2S,3S) could also be accomplished from the syn-L-isomer (2S,3R) using the synthetic procedure as presented in FIG. 2 for compound 509, which is based upon a similar transformation used in the synthesis of the natural product ustiloxin D (Wandless, T. J.; et al. J. Am. Chem. Soc. 2003, 115, 6864-6865).

### B. Tethers

Tethers were obtained from the methods previously described in Intl. Pat. Appl. WO 01/25257, WO 2004/111077, WO 2005/012331, WO 2006/009645 and WO 2006/009674.

Exemplary tethers (T) include, but are not limited to, the following:

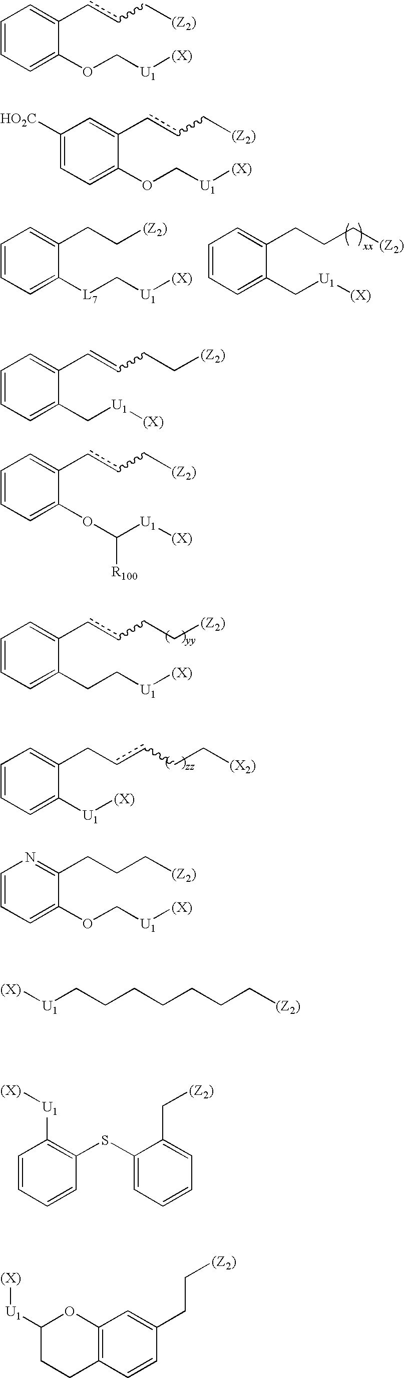

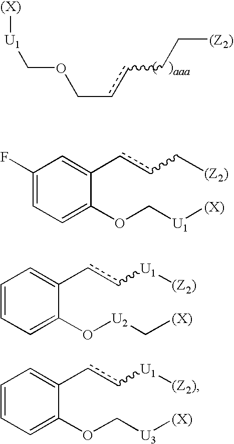

and intermediates in the manufacture thereof, wherein (Z₂) is the site of a covalent bond of T to Z₂, and Z₂ is defined above, and wherein (X) is the site of a covalent bond of T to X, and X is defined above; L₇ is —CH₂— or —O—; U₁ and U₃ are each independently —CR₁₀₁R₁₀₂— or —C(═O)—; U₂ is —CR₁₀₁R₁₀₂—; R₁₀₀ is lower alkyl; R₁₀₁ and R₁₀₂ are each independently hydrogen, lower alkyl or substituted lower alkyl; xx is 2 or 3; yy is 1 or 2; zz is 1 or 2; and aaa is 0 or 1.

### C. Solid Phase and Solution Phase Techniques

Specific solid phase techniques for the synthesis of the macrocyclic compounds of the invention have been described in WO 01/25257, WO 2004/111077, WO 2005/012331 and WO 2005/012332. Solution phase synthesis routes, including methods amenable to larger scale manufacture, were described in International Patent Application Publication Nos. WO 2006/009645 and WO 2006/009674.

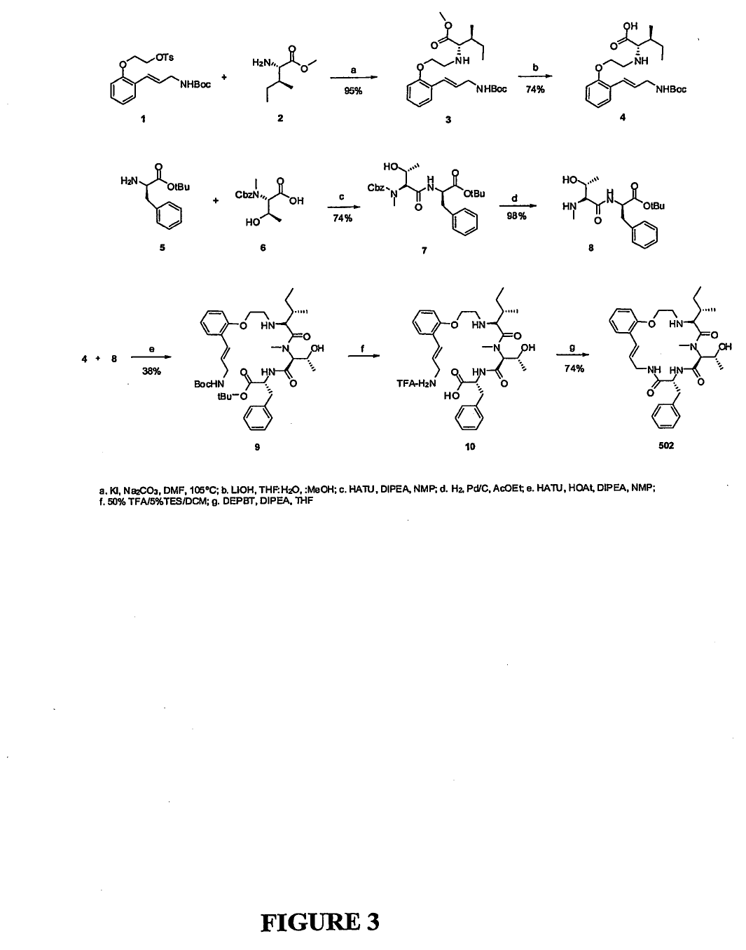

In particular, macrocyclic compounds of the invention can be made using a solution phase procedure as illustrated for representative compound 502 in FIG. 3. Synthetic yields for representative compounds of the invention are presented in Table 1.

TABLE 1
Synthetic Results for Representative Compounds of the Invention

| Compound | Structure | Yield |
|---|---|---|
| 152 | 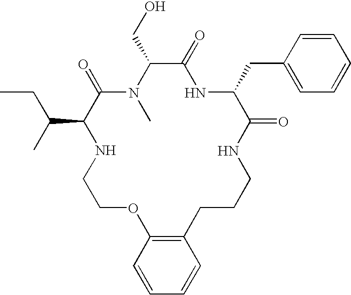 | 22.0% |
| 502 | 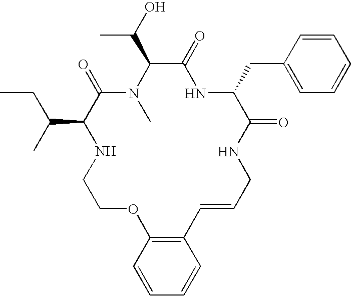 | 20.3% |
| 503 | 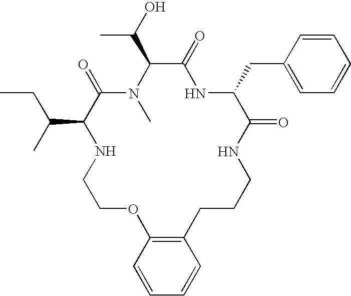 | 20.0% |
| 504 | 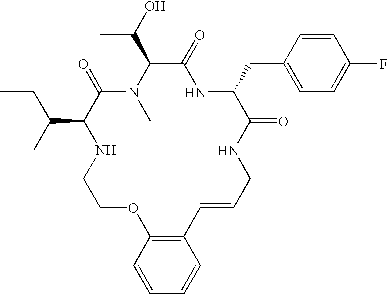 | 6.5% |
| 505 | 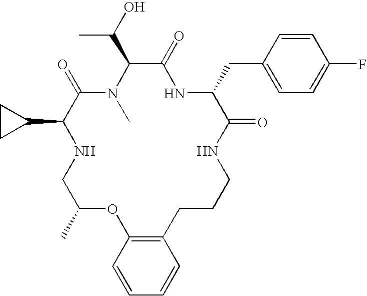 | 35.0% |
| 506 | 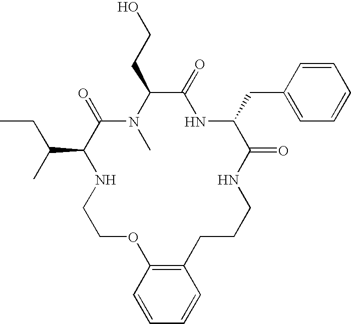 | 7.3% |
| 507 | 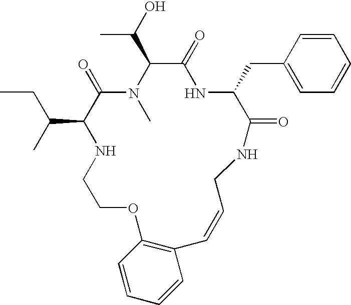 | 8.8% |
| 508 | 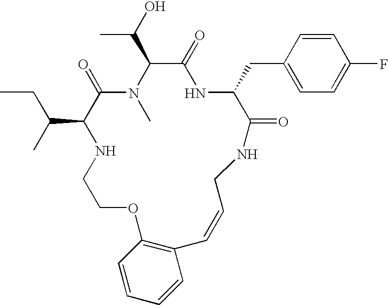 | 7.3% |
| 509 | 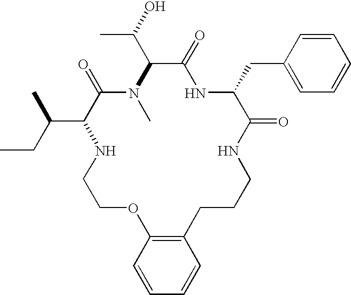 | 3.2% |

### D. Analytical Methods

Specific analytical techniques for the characterization of the macrocyclic compounds of the invention have been described in WO 01/25257, WO 2004/111077, WO 2005/012331 and WO 2005/012332.

Analytical data for some representative compounds of the invention are summarized in Table 2.

TABLE 2
Analytical Data for Representative Compounds of the Invention

| | Molecular | | |
|---|---|---|---|
| Compound | Formula | Molecular Weight | HPLC-MS |
| 152 | C₃₀H₄₂N₄O₅ | 538.7 | 539 |
| 502 | C₃₁H₄₂N₄O₅ | 550.7 | 551 |
| 503 | C₃₁H₄₄N₄O₅ | 552.7 | 553 |
| 504 | C₃₁H₄₁FN₄O₅ | 568.7 | 569 |
| 505 | C₃₁H₄₁FN₄O₅ | 568.7 | 569 |
| 506 | C₃₁H₄₄N₄O₅ | 552.70 | 553 |
| 507 | C₃₁H₄₂N₄O₅ | 550.69 | 551 |
| 508 | C₃₁H₄₁FN₄O₅ | 568.7 | 569 |
| 509 | C₃₁H₄₄N₄O₅ | 552.7 | 553 |

### 3. Biological Methods

The compounds of the present invention were evaluated for their ability to interact at the human ghrelin receptor utilizing a competitive radioligand binding assay, fluorescence assay or Aequorin functional assay as described in the examples below. Such methods can be conducted, if so desired, in a high throughput manner to permit the simultaneous evaluation of many compounds.

Specific assay methods for the human (GHS-R1a), swine and rat GHS-receptors (U.S. Pat. No. 6,242,199, Intl. Pat. Appl. Nos. WO 97/21730 and 97/22004), as well as the canine GHS-receptor (U.S. Pat. No. 6,645,726), and their use in generally identifying agonists and antagonists thereof are known.

Functional ghrelin antagonists can be identified utilizing the methods described in WO 2005/114180, while inverse agonists of the receptor can be assayed using the methods of WO 2004/056869.

Appropriate methods for determining the functional activity of compounds of the present invention that interact at the human ghrelin receptor are also described in the Examples below.

The in vivo efficacy of compounds of the present invention can be illustrated, for example, using animal models of obesity such as those described in the literature (WO 2004/056869; Nakazato, M.; Murakami, N.; Date, Y.; et al. A role for ghrelin in the central regulation of feeding. Nature 2001, 409, 194-198; Murakami, N.; Hayashida, T.; Kuroiwa, T.; et al. Role for central ghrelin in food intake and secretion profile of stomach ghrelin in rats. J. Endocrinol. 2002, 174, 283-288; Asakawa, A.; Inui, A.; Kaga, T.; et al. Antagonism of ghrelin receptor reduces food intake and body weight gain in mice. Gut 2003, 52, 947-952; Sun, Y.; Ahmed, S.; Smith, R. G. Deletion of ghrelin impairs neither growth nor appetite. Mol. Cell. Biol. 2003, 23, 7973-7981; Wortley, K. E.; Anderson, K. D.; Garcia, K.; et al. Genetic deletion of ghrelin does not decrease food intake but influences metabolic fuel preferences. Proc. Natl. Acad. Sci. USA 2004, 101, 8227-8232; Halem, H. A.; Taylor, J. E.; Dong, J. Z.; Shen, Y.; Datta, R.; Abizaid, A.; Diano, S.; Horvath, T.; Zizzari, P.; Bluet-Pajot, M.-T.; Epelbaum, J.; Culler, M. D. Novel analogs of ghrelin: physiological and clinical implications. Eur. J. Endocrinol. 2004, 151, S71-S75; Helmling, S.; Maasch, C.; Eulberg, D.; et al. Inhibition of ghrelin action in vitro and in vivo by an RNA-Spiegelmer. Proc. Natl. Acad. Sci. USA 2004, 101, 13174-13179; Shearman, L. P.; Wang, S. P.; Helmling, S.; et al. Ghrelin neutralization by a ribonucleic acid-SPM ameliorates obesity in diet-induced obese mice. Endocrinology 2006, 147, 1517-1526).

### 4. Pharmaceutical Compositions

The macrocyclic compounds of the present invention or pharmacologically acceptable salts thereof according to the invention may be formulated into pharmaceutical compositions of various dosage forms. To prepare the pharmaceutical compositions of the invention, one or more compounds, including optical isomers, enantiomers, diastereomers, racemates or stereochemical mixtures thereof, or pharmaceutically acceptable salts thereof as the active ingredient is intimately mixed with appropriate carriers and additives according to techniques known to those skilled in the art of pharmaceutical formulations.

A pharmaceutically acceptable salt refers to a salt form of the compounds of the present invention in order to permit their use or formulation as pharmaceuticals and which retains the biological effectiveness of the free acids and bases of the specified compound and that is not biologically or otherwise undesirable. Examples of such salts are described in Handbook of Pharmaceutical Salts: Properties, Selection, and Use, Wermuth, C. G. and Stahl, P. H. (eds.), Wiley-Verlag Helvetica Acta, Zürich, 2002 [ISBN 3-906390-26-8]. Examples of such salts include alkali metal salts and addition salts of free acids and bases. Examples of pharmaceutically acceptable salts, without limitation, include sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, xylenesulfonates, phenylacetates, phenylpropionates, phenylbutyrates, citrates, lactates, γ-hydroxybutyrates, glycollates, tartrates, methanesulfonates, ethane sulfonates, propanesulfonates, toluenesulfonates, naphthalene-1-sulfonates, naphthalene-2-sulfonates, and mandelates.

If an inventive compound is a base, a desired salt may be prepared by any suitable method known to those skilled in the art, including treatment of the free base with an inorganic acid, such as, without limitation, hydrochloric acid, hydrobromic acid, hydroiodic, carbonic acid, sulfuric acid, nitric acid, phosphoric acid, and the like, or with an organic acid, including, without limitation, formic acid, acetic acid, propionic acid, maleic acid, succinic acid, mandelic acid, fumaric acid, malonic acid, pyruvic acid, oxalic acid, stearic acid, ascorbic acid, glycolic acid, salicylic acid, pyranosidyl acid, such as glucuronic acid or galacturonic acid, alpha-hydroxy acid, such as citric acid or tartaric acid, amino acid, such as aspartic acid or glutamic acid, aromatic acid, such as benzoic acid or cinnamic acid, sulfonic acid, such as p-toluenesulfonic acid, methanesulfonic acid, ethanesulfonic acid, 2-hydroxyethanesulfonic acid, benzenesulfonic acid, cyclohexylaminosulfonic acid or the like.

If an inventive compound is an acid, a desired salt may be prepared by any suitable method known to the art, including treatment of the free acid with an inorganic or organic base, such as an amine (primary, secondary, or tertiary); an alkali metal or alkaline earth metal hydroxide; or the like. Illustrative examples of suitable salts include organic salts derived from amino acids such as glycine, lysine and arginine; ammonia, primary, secondary, and tertiary amines such as ethylenediamine, N,N′-dibenzylethylenediamine, diethanolamine, choline, and procaine, and cyclic amines, such as piperidine, morpholine, and piperazine; as well as inorganic salts derived from sodium, calcium, potassium, magnesium, manganese, iron, copper, zinc, aluminum, and lithium.

The carriers and additives used for such pharmaceutical compositions can take a variety of forms depending on the anticipated mode of administration. Thus, compositions for oral administration may be, for example, solid preparations such as tablets, sugar-coated tablets, hard capsules, soft capsules, granules, powders and the like, with suitable carriers and additives being starches, sugars, binders, diluents, granulating agents, lubricants, disintegrating agents and the like. Because of their ease of use and higher patient compliance, tablets and capsules represent the most advantageous oral dosage forms for many medical conditions.

Similarly, compositions for liquid preparations include solutions, emulsions, dispersions, suspensions, syrups, elixirs, and the like with suitable carriers and additives being water, alcohols, oils, glycols, preservatives, flavoring agents, coloring agents, suspending agents, and the like. Typical preparations for parenteral administration comprise the active ingredient with a carrier such as sterile water or parenterally acceptable oil including polyethylene glycol, polyvinyl pyrrolidone, lecithin, arachis oil or sesame oil, with other additives for aiding solubility or preservation may also be included. In the case of a solution, it can be lyophilized to a powder and then reconstituted immediately prior to use. For dispersions and suspensions, appropriate carriers and additives include aqueous gums, celluloses, silicates or oils.

The pharmaceutical compositions according to embodiments of the present invention include those suitable for oral, rectal, topical, inhalation (e.g., via an aerosol) buccal (e.g., sub-lingual), vaginal, topical (i.e., both skin and mucosal surfaces, including airway surfaces), transdermal administration and parenteral (e.g., subcutaneous, intramuscular, intradermal, intraarticular, intrapleural, intraperitoneal, intrathecal, intracerebral, intracranially, intraarterial, or intravenous), although the most suitable route in any given case will depend on the nature and severity of the condition being treated and on the nature of the particular active agent which is being used.

Compositions for injection will include the active ingredient together with suitable carriers including propylene glycol-alcohol-water, isotonic water, sterile water for injection (USP), emulPhor™-alcohol-water, cremophor-EL™ or other suitable carriers known to those skilled in the art. These carriers may be used alone or in combination with other conventional solubilizing agents such as ethanol, propylene glycol, or other agents known to those skilled in the art.

Where the macrocyclic compounds of the present invention are to be applied in the form of solutions or injections, the compounds may be used by dissolving or suspending in any conventional diluent. The diluents may include, for example, physiological saline, Ringer's solution, an aqueous glucose solution, an aqueous dextrose solution, an alcohol, a fatty acid ester, glycerol, a glycol, an oil derived from plant or animal sources, a paraffin and the like. These preparations may be prepared according to any conventional method known to those skilled in the art.

Compositions for nasal administration may be formulated as aerosols, drops, powders and gels. Aerosol formulations typically comprise a solution or fine suspension of the active ingredient in a physiologically acceptable aqueous or non-aqueous solvent. Such formulations are typically presented in single or multidose quantities in a sterile form in a sealed container. The sealed container can be a cartridge or refill for use with an atomizing device. Alternatively, the sealed container may be a unitary dispensing device such as a single use nasal inhaler, pump atomizer or an aerosol dispenser fitted with a metering valve set to deliver a therapeutically effective amount, which is intended for disposal once the contents have been completely used. When the dosage form comprises an aerosol dispenser, it will contain a propellant such as a compressed gas, air as an example, or an organic propellant including a fluorochlorohydrocarbon or fluorohydrocarbon.

Compositions suitable for buccal or sublingual administration include tablets, lozenges and pastilles, wherein the active ingredient is formulated with a carrier such as sugar and acacia, tragacanth or gelatin and glycerin.

Compositions for rectal administration include suppositories containing a conventional suppository base such as cocoa butter.

Compositions suitable for transdermal administration include ointments, gels and patches.

Other compositions known to those skilled in the art can also be applied for percutaneous or subcutaneous administration, such as plasters.

Further, in preparing such pharmaceutical compositions comprising the active ingredient or ingredients in admixture with components necessary for the formulation of the compositions, other conventional pharmacologically acceptable additives may be incorporated, for example, excipients, stabilizers, antiseptics, wetting agents, emulsifying agents, lubricants, sweetening agents, coloring agents, flavoring agents, isotonicity agents, buffering agents, antioxidants and the like. As the additives, there may be mentioned, for example, starch, sucrose, fructose, lactose, glucose, dextrose, mannitol, sorbitol, precipitated calcium carbonate, crystalline cellulose, carboxymethylcellulose, dextrin, gelatin, acacia, EDTA, magnesium stearate, talc, hydroxypropylmethylcellulose, sodium metabisulfite, and the like.

In some embodiments, the composition is provided in a unit dosage form such as a tablet or capsule.

In further embodiments, the present invention provides kits including one or more containers comprising pharmaceutical dosage units comprising an effective amount of one or more compounds of the present invention.

The present invention further provides prodrugs comprising the compounds described herein. The term “prodrug” is intended to mean a compound that is converted under physiological conditions or by solvolysis or metabolically to a specified compound that is pharmaceutically active. The “prodrug” can be a compound of the present invention that has been chemically derivatized such that, (i) it retains some, all or none of the bioactivity of its parent drug compound, and (ii) it is metabolized in a subject to yield the parent drug compound. The prodrug of the present invention may also be a “partial prodrug” in that the compound has been chemically derivatized such that, (i) it retains some, all or none of the bioactivity of its parent drug compound, and (ii) it is metabolized in a subject to yield a biologically active derivative of the compound. Known techniques for derivatizing compounds to provide prodrugs can be employed. Such methods may utilize formation of a hydrolyzable coupling to the compound.

The present invention further provides that the compounds of the present invention may be administered in combination with a therapeutic agent used to prevent and/or treat metabolic and/or endocrine disorders, cardiovascular disorders, obesity and obesity-associated disorders, gastrointestinal disorders, genetic disorders, hyperproliferative disorders and inflammatory disorders. Exemplary agents include analgesics including opioid analgesics, anesthetics, antifungals, antibiotics, antiinflammatories, including nonsteroidal anti-inflammatory agents, anthelmintics, antiemetics, antihistamines, antihypertensives, antipsychotics, antiarthritics, antitussives, antivirals, cardioactive drugs, cathartics, chemotherapeutic agents such as DNA-interactive agents, antimetabolites, tubulin-interactive agents, hormonal agents, and agents such as asparaginase or hydroxyurea, corticoids (steroids), antidepressants, depressants, diuretics, hypnotics, minerals, nutritional supplements, parasympathomiimetics, hormones such as corticotrophin releasing hormone, adrenocorticotropin, growth hormone releasing hormone, growth hormone, thyrptropin-releasing hormone and thyroid stimulating hormone, sedatives, sulfonamides, stimulants, sympathomimetics, tranquilizers, vasoconstrictors, vasodilators, vitamins and xanthine derivatives.

Subjects suitable to be treated according to the present invention include, but are not limited to, avian and mammalian subjects, and are preferably mammalian. Mammals of the present invention include, but are not limited to, canines, felines, bovines, caprines, equines, ovines, porcines, rodents (e.g. rats and mice), lagomorphs, primates, humans, and the like, and mammals in utero. Any mammalian subject in need of being treated according to the present invention is suitable. Human subjects are preferred. Human subjects of both genders and at any stage of development (i.e., neonate, infant, juvenile, adolescent, adult) can be treated according to the present invention.

Illustrative avians according to the present invention include chickens, ducks, turkeys, geese, quail, pheasant, ratites (e.g., ostrich) and domesticated birds (e.g., parrots and canaries), and birds in ovo.

The present invention is primarily concerned with the treatment of human subjects, but the invention can also be carried out on animal subjects, particularly mammalian subjects such as mice, rats, dogs, cats, livestock and horses for veterinary purposes, and for drug screening and drug development purposes.

In therapeutic use for treatment of conditions in mammals (i.e. humans or animals) for which an antagonist or inverse agonist of the ghrelin receptor is effective, the compounds of the present invention or an appropriate pharmaceutical composition thereof may be administered in an effective amount. Since the activity of the compounds and the degree of the therapeutic effect vary, the actual dosage administered will be determined based upon generally recognized factors such as age, condition of the subject, route of delivery and body weight of the subject. The dosage will be from about 0.1 to about 100 mg/kg, administered orally 1-4 times per day. In addition, compounds may be administered by injection at approximately 0.01-20 mg/kg per dose, with administration 1-4 times per day. Treatment could continue for weeks, months or longer. Determination of optimal dosages for a particular situation is within the capabilities of those skilled in the art.

### 5. Methods of Use

The compounds of the present invention can be used for the prevention and treatment of a range of medical conditions including, but not limited to, metabolic and/or endocrine disorders, cardiovascular disorders, obesity and obesity-associated disorders, gastrointestinal disorders, genetic disorders, hyperproliferative disorders, inflammatory disorders and combinations thereof where the disorder may be the result of multiple underlying maladies.

Metabolic and/or endocrine disorders include, but are not limited to, obesity and diabetes, in particular, type II diabetes. Cardiovascular disorders include, but are not limited to, hypertension and dyslipidemia. Hyperproliferative disorders include, but are not limited to, tumors, cancers, and neoplastic tissue, which further include disorders such as breast cancers, osteosarcomas, angiosarcomas, fibrosarcomas and other sarcomas, leukemias, lymphomas, sinus tumors, ovarian, uretal, bladder, prostate and other genitourinary cancers, colon, esophageal and stomach cancers and other gastrointestinal cancers, lung cancers, myelomas, pancreatic cancers, liver cancers, kidney cancers, endocrine cancers, skin cancers, and brain or central and peripheral nervous (CNS) system tumors, malignant or benign, including gliomas and neuroblastomas. Obesity and obesity-associated disorders include, but are not limited to, retinopathy, hyperphasia and disorders involving regulation of food intake and appetite control in addition to obesity being characterized as a metabolic and/or endocrine disorder. Gastrointestinal disorders include, but are not limited to, irritable bowel syndrome, dyspepsia, opioid-induced bowel dysfunction and gastroparesis. Inflammatory disorders include, but are not limited to, general inflammation, arthritis, for example, rheumatoid arthritis and osteoarthritis, and inflammatory bowel disease. The compounds of the present invention can further be used to prevent and/or treat cirrhosis and chronic liver disease. As used herein, “treatment” is not necessarily meant to imply cure or complete abolition of the disorder or symptoms associated therewith.

The compounds of the present invention can further be utilized for the preparation of a medicament for the treatment of a range of medical conditions including, but not limited to, metabolic and/or endocrine disorders, cardiovascular disorders, obesity and obesity-associated disorders, gastrointestinal disorders, genetic disorders, hyperproliferative disorders and inflammatory disorders.

Further embodiments of the present invention will now be described with reference to the following examples. It should be appreciated that these examples are for the purposes of illustrating embodiments of the present invention, and do not limit the scope of the invention.

### EXAMPLE 1

### Competitive Radioligand Binding Assay

### Ghrelin Receptor

The competitive binding assay at the human growth hormone secretagogue receptor (HGHS-R1a) was carried out analogously to assays described in the literature. (Bednarek M A et al. Structure-function studies on the new growth hormone-releasing peptide ghrelin: minimal sequence of ghrelin necessary for activation of growth hormone secretagogue receptor 1a; J. Med. Chem. 2000, 43, 4370-4376; Palucki, B. L. et al. Spiro(indoline-3,4′-piperidine) growth hormone secretagogues as ghrelin mimetics; Bioorg. Med. Chem. Lett. 2002, 11, 1955-1957).

### Materials

Membranes (GHS-R/HEK 293) were prepared from HEK-293 cells stably transfected with the human ghrelin receptor (hGHS-R1a). These membranes were provided by PerkinElmer BioSignal (#RBHGHSM, lot#1887) and utilized at a quantity of 0.71 μg/assay point.
1. [¹²⁵]-Ghrelin (PerkinElmer, #NEX-388); final concentration: 0.0070-0.0085 nM
2. Ghrelin (Bachem, #H-4864); final concentration: 1 μM
3. Multiscreen Harvest plates-GF/C (Millipore, #MAHFC1H60)
4. Deep-well polypropylene titer plate (Beckman Coulter, #267006)
5. TopSeal-A (PerkinElmer, #6005185)
6. Bottom seal (Millipore, #MATAH0P00)
7. MicroScint-0 (PerkinElmer, #6013611)
8. Binding Buffer: 25 mM Hepes (pH 7.4), 1 mM CaCl₂, 5 mM MgCl₂, 2.5 mM EDTA, 0.4% BSA

### Assay Volumes

Competition experiments were performed in a 300 μl filtration assay format.
1. 220 μL of membranes diluted in binding buffer
2. 40 μL of compound diluted in binding buffer
3. 40 μl of radioligand ([¹²⁵I]-Ghrelin) diluted in binding buffer
   Final test concentrations (N=1) for compounds of the present invention: 10, 1, 0.5, 0.2, 0.1, 0.05, 0.02, 0.01, 0.005, 0.002, 0.001 μM.

### Compound Handling

Compounds were provided frozen on dry ice at a stock concentration of 10 mM diluted in 100% DMSO and stored at −80° C. until the day of testing. On the test day, compounds were allowed to thaw at rt overnight and then diluted in assay buffer according to the desired test concentrations. Under these conditions, the maximal final DMSO concentration in the assay was 0.1%.

### Assay Protocol

In deep-well plates, 220 μL of diluted cell membranes (final concentration: 0.71 μg/well) were combined with 40 μL of either binding buffer (total binding, N=5), 1 μM ghrelin (non-specific binding, N=3) or the appropriate concentration of test compound N=2 for each test concentration). The reaction was initiated by addition of 40 μL of [¹²⁵I]-ghrelin (final conc. 0.0070-0.0085 nM) to each well. Plates were sealed with TopSeal-A, vortexed gently and incubated at rt for 30 min. The reaction was arrested by filtering samples through Multiscreen Harvest plates (pre-soaked in 0.5% polyethyleneimine) using a Tomtec Harvester, washed 9 times with 500 μL of cold 50 mM Tris-HCl (pH 7.4, 4° C.), and then plates were air-dried in a fumehood for 30 min. A bottom seal was applied to the plates prior to the addition of 25 μL of MicroScint-0 to each well. Plates were than sealed with TopSeal-A and counted for 30 sec per well on a TopCount Microplate Scintillation and Luminescence Counter (PerkinElmer) using a count delay of 60 sec. Results were expressed as counts per minute (cpm).

Data were analyzed by GraphPad Prism (GraphPad Software, San Diego, Calif.) using a variable slope non-linear regression analysis. Kᵢ values were calculated using a K_{d} value of 0.01 nM for [¹²⁵I]-ghrelin (previously determined during membrane characterization).

Dₘₐₓ values were calculated using the following formula:

$${D}_{max} = 1 - \frac{\begin{matrix}test concentration with maximal \\ displacement - non \text{-} specific binding\end{matrix}}{total binding - non \text{-} specific binding} \times 100$$

where total and non-specific binding represent the cpm obtained in the absence or presence of 1 μM ghrelin, respectively.

Results for the examination of representative compounds of the present invention are presented below in Table 3.

TABLE 3
Binding Activity for Representative Compounds of the Invention

| | | Activity |
|---|---|---|
| Compound | Structure | (Kᵢ,)* |
| 2 | 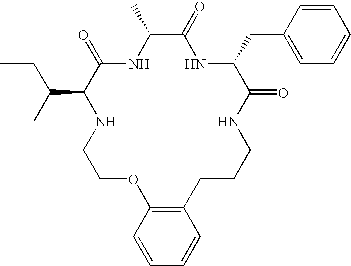 | B |
| 14 | 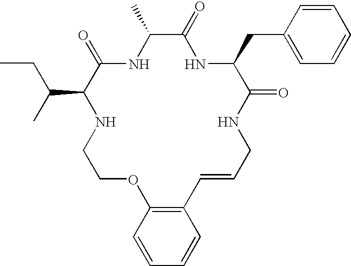 | B |
| 59 | 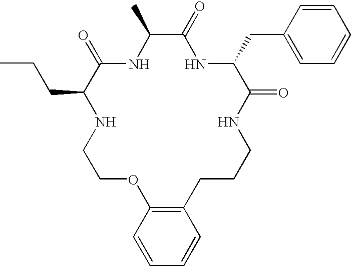 | C |
| 60 | 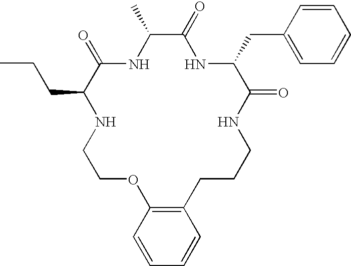 | B |
| 79 | 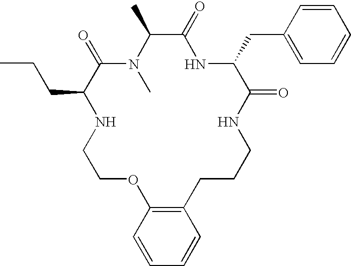 | B |
| 93 | 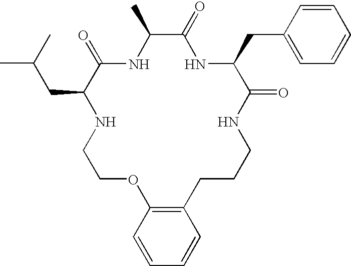 | C |
| 94 | 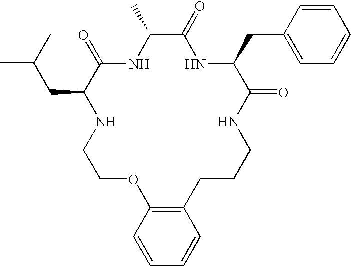 | C |
| 151 | 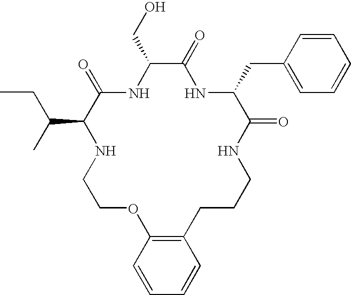 | C |
| 152 | 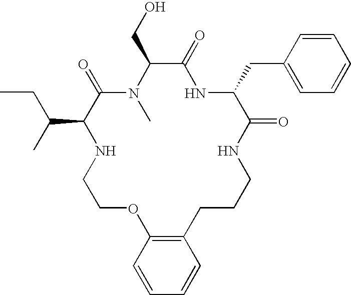 | B |
| 215 | 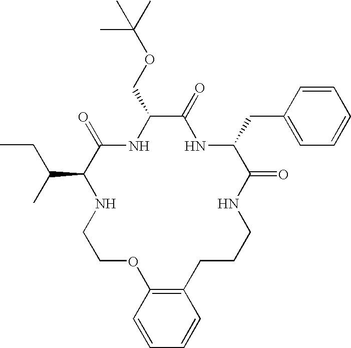 | C |
| 501 | 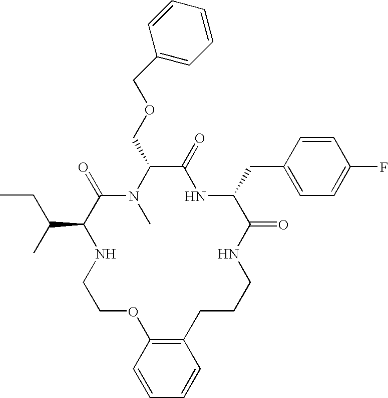 | B |
| 502 | 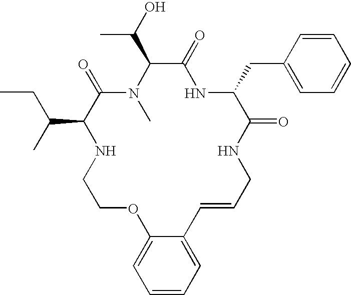 | A |
| 503 | 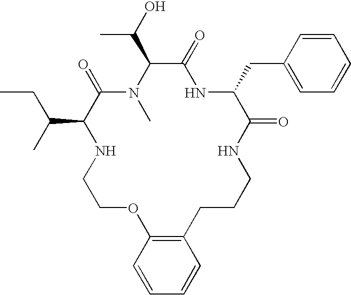 | A |
| 504 | 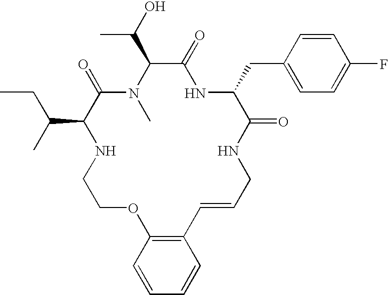 | A |
| 505 | 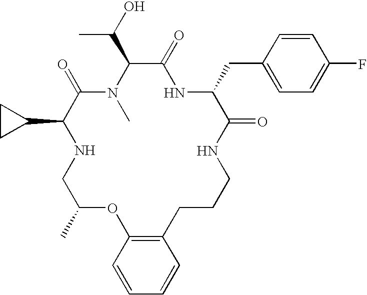 | B |
| 506 | 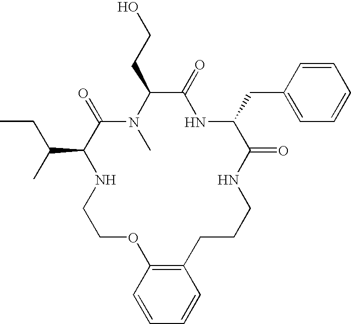 | B |
| 507 | 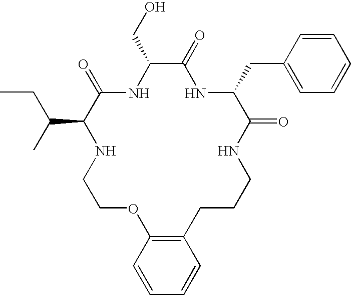 | B |
| 508 | 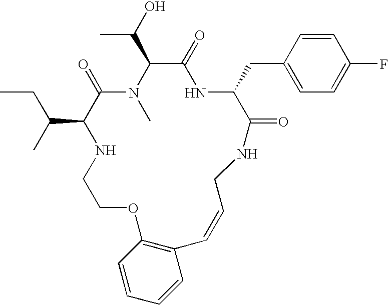 | A |
| 509 | 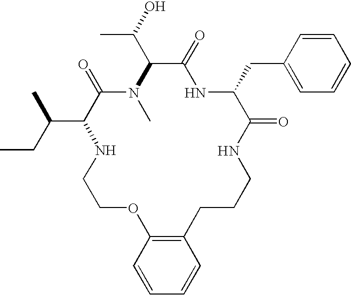 | B |

*Activity is expressed as follows: A: Kᵢ = 1-100 nM, B: Kᵢ = 100-1000 nM, C: Kᵢ = 1000-10,000 nM.

### EXAMPLE 2

### Fluorescence Functional Assay

### Ghrelin Receptor

### Equipment

1. ImageTrak Epi-Fluorescence system (Perkin-Elmer)
2. MultiDrop TiterTek
3. CO₂ incubators: 5% CO₂, humidified, 37° C.

### Materials

1. Hanks' BSS without phenol red (Life Technologies)
2. Hepes buffer
3. Probenecid (Sigma)
4. FLIPR Calcium-3 Assay Kit (Molecular Devices #R-8091)
5. Falcon cell culture 96-well black/clear bottom plates
6. 0.05% trypsin-EDTA
7. Cells: HEK293 cells expressing GHS-R1a receptor (Perkin-Elmer BioSignal) were grown in DMEM (Dulbecco's Modified Eagles Medium) with 10% FBS, 1% sodium pyruvate, 1% NEAA and 400 μg/mL geneticin
8. Ghrelin (reference agonist; Bachem, #H-4864)
9. [D-Lys³]-GHRP-6 (reference antagonist, Phoenix #031-22)
10. Assay buffer: HBSS—20 mM Hepes containing 2.5 mM probenecid and 0.1% BSA (bovine serum albumin); pH 7.4

### Compound Handling

Stock solutions of compounds (10 mM in 100% DMSO) were provided frozen on dry ice and stored at −80° C. prior to use. From the stock solution, mother solutions were made at a concentration of 100 μM by 100-fold dilution in 26% DMSO. Assay plates were then prepared by appropriate dilution in assay buffer.

Final Test Concentrations (N=10) for Test Compounds (agonist):

### 1, 0.3, 0.1, 0.03, 0.01, 0.003, 0.001, 0.0003, 0.0001, 0.00003 μM.

Final Test Concentrations (N=10) for Test Compounds (antagonist):

### 10, 3, 1, 0.3, 0.1, 0.03, 0.01, 0.003, 0.001, 0.0003 μM.

### Cell Preparation

Cells were maintained in culture as indicated above. The cells were harvested at a confluency of 70-90% the day before the experiment. Growth medium was removed and the cells rinsed briefly with PBS without Ca⁺² and Mg⁺². 0.05% Trypsin was added and the plates incubated at 37° C. for 5 min to detach the cells. DMEM medium supplemented with 10% FBS was added to inactivate the trypsin and determine the cell concentration. The inoculum was adjusted to a final concentration of 200 cells/mL and dispensed at 200 μL per well into a 96-well block plate. The plates were incubated at 37° C. overnight. The cellular confluence must be between 70-95% on the day of the experiment.

### Assay Protocol

The plates were removed from the incubator and the media removed by inversion of the plates. Calcium-3 dye, 50 μL, was loaded and then incubated for 1 h at 37° C. The plate was again inverted and then 25 μL of assay buffer added. The plates were then transferred to the ImageTrak system for analysis. For agonist testing, after reading for ten (10) sec, 25 μL of 2× test compound or control was injected into the assay plate. Fluorescence was monitored for an additional 50 sec. A reading was taken every two (2) seconds for a total of 30 readings per assay point.

For antagonist testing, after reading for ten (10) sec, 12.5 μL of 3× test compound or control was injected into the assay plate and allowed to react for three (3) min. At that time, 4 nM ghrelin (corresponds to EC₈₀) was injected and fluorescence was monitored for an additional 60 sec. A reading was taken every two (2) seconds for a total of 125 readings per data point.

### Analysis and Expression of Results

For agonists, values obtained for each assay point reflect Max-Min of fluorescence readings where Max represents the maximal value of the 30 readings taken and Min represents the minimum value observed before injection of the compound from the first five readings. Concentration response curves were analyzed using GraphPad Prism (GraphPad Software, San Diego, Calif.) by non-linear regression analysis (sigmoidal dose-response). EC₅₀ values are calculated using GraphPad.

Eₘₐₓ values were calculated using the following formula:

$${E}_{max} = \frac{ \begin{matrix}counts at the concentration of compound \\ with maximum response - Basal\end{matrix} }{Ago ( {E}_{max} ) - Basal} \times 100$$

where Basal and Ago(Eₘₐₓ), represent the average counts obtained in the absence or presence of 1 μM ghrelin, respectively.

For antagonists, values obtained for each assay point reflect Max-Min of fluorescence readings where Max represents the maximal value obtained after injection of ghrelin at EC₈₀ and Min represents the minimum value observed before injection of the compound from the first five readings. Concentration response curves were analyzed using GraphPad Prism (GraphPad Software, San Diego, Calif.) by non-linear regression analysis (sigmoidal dose-response). IC₅₀ values are calculated using GraphPad.

Iₘₐₓ values were calculated using the following formula:

$${I}_{max} = \frac{ \begin{matrix}counts at concentration of compound \\ with maximum response - Ago ( {EC}_{80} )\end{matrix} }{Basal - Ago ( {EC}_{80} )} \times 100$$

where Basal and Ago(EC₈₀) represent the average counts obtained in the absence or presence of 5 nM ghrelin at the second addition step, respectively.

### EXAMPLE 3

### Aequorin Functional Assay

### Ghrelin Receptor

The functional activity of compounds of the invention found to bind to the GHS-R1a receptor can be determined using the method described below. (LePoul, E.; et al. Adaptation of aequorin functional assay to high throughput screening. J. Biomol. Screen. 2002, 7, 57-65; Bednarek, M. A.; et al. Structure-function studies on the new growth hormone-releasing peptide ghrelin: minimal sequence of ghrelin necessary for activation of growth hormone secretagogue receptor 1a. J. Med. Chem. 2000, 43, 4370-4376; Palucki, B. L.; et al. Spiro(indoline-3,4′-piperidine) growth hormone secretagogues as ghrelin mimetics. Bioorg. Med. Chem. Lett. 2001, 11, 1955-1957).

### Materials

Membranes were prepared using AequoScreen™ (EUROSCREEN, Belgium) cell lines expressing the human ghrelin receptor (cell line ES-410-A; receptor accession #60179). This cell line is constructed by transfection of the human ghrelin receptor into CHO-K1 cells co-expressing G_{α16} and the mitochondrially targeted Aequorin (Ref #ES-WT-A5).
1. Ghrelin (reference agonist; Bachem, #H-4864)
2. Assay buffer: DMEM (Dulbecco's Modified Eagles Medium) containing 0.1% BSA (bovine serum albumin; pH 7.0.
3. Coelenterazine (Molecular Probes, Leiden, The Netherlands)

### Final Test Concentrations (N=8) for Compounds:

### 10, 1, 0.3, 0.1, 0.03, 0.01, 0.003, 0.001 μM.

### Compound Handling

Stock solutions of compounds (10 mM in 100% DMSO) were provided frozen on dry ice and stored at −20° C. prior to use. From the stock solution, mother solutions were made at a concentration of 500 μM 20-fold dilution in 26% DMSO. Assay plates were then prepared by appropriate dilution in DMEM medium containing 0.1% BSA. Under these conditions, the maximal final DMSO concentration in the assay was <0.6%.

### Cell Preparation

AequoScreen™ cells were collected from culture plates with Ca²⁺ and Mg²⁺-free phosphate buffered saline (PBS) supplemented with 5 mM EDTA, pelleted for 2 minutes at 1000×g, re-suspended in DMEM-Ham's F12 containing 0.1% BSA at a density of 5×10⁶ cells/ml and incubated overnight at room temperature in the presence of 5 μM coelenterazine. After loading, cells were diluted with assay buffer to a concentration of 5×10⁵ cells/ml.

### Assay Protocol

For agonist testing, 50 μl of the cell suspension was mixed with 50 μl of the appropriate concentration of test compound or ghrelin (reference agonist) in 96-well plates (duplicate samples). Ghrelin (reference agonist) is tested at several concentrations concurrently with the test compounds in order to validate the experiment. The emission of light resulting from receptor activation in response to ghrelin or test compounds was recorded using the Hamamatsu FDSS 6000 reader (Hamamatsu Photonics K.K., Japan).

For antagonist testing, an approximate EC₈₀ concentration of ghrelin (i.e. 3.7 nM; 100 μL) was injected onto the cell suspension containing the test compounds (duplicate samples) 15-30 minutes after the end of agonist testing and the consequent emission of light resulting from receptor activation was measured as described in the paragraph above.

### Analysis and Expression of Results

Results are expressed as Relative Light Units (RLU). Concentration response curves were analyzed using GraphPad Prism (GraphPad Software, San Diego, Calif.) by non-linear regression analysis (sigmoidal dose-response) based on the equation E=Eₘₐₓ/(1+EC₅₀/c)n where E is the measured RLU value at a given agonist concentration (C), Eₘₐₓ is the maximal response, EC₅₀ is the concentration producing 50% stimulation and n is the slope index. For agonist testing, results for each concentration of test compound were expressed as percent activation relative to the signal induced by ghrelin at a concentration equal to the EC₈₀ (i.e. 3.7 nM). EC₅₀, Hill slope and % Eₘₐₓ values are reported.

For antagonist testing, results for each concentration of test compound were expressed as percent inhibition relative to the signal induced by ghrelin at a concentration equal to the EC₈₀.

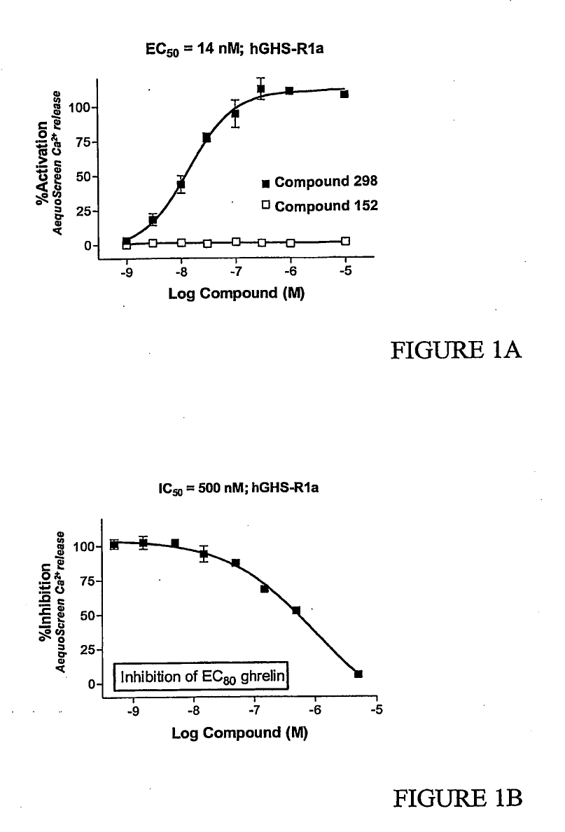

In the assay of Example 3, representative compounds of the invention demonstrated activity as provided in Table 4. Results for compound 152 are shown in FIG. 1. Further, compound 152 demonstrates no receptor activation in comparison to known ghrelin agonists. In the assay of Example 2, compound 502 produced a functional Kᵢ of level B.

TABLE 4
Functional Activity of Representative Compounds of the Invention

| | Activity |
|---|---|
| Compound | (IC₅₀)* |
| 152 | B |
| 502 | A |
| 503 | A |
| 504 | A |
| 505 | B |
| 506 | B |
| 507 | C |
| 508 | B |
| 509 | C |

*Activity is expressed as follows: A: IC₅₀ = 1-100 nM, B: IC₅₀ = 100-1500 nM, C: IC₅₀ > 1500 nM

### EXAMPLE 4

### Inverse Agonist Assay

The inverse agonist activity at the ghrelin receptor for compounds of the invention can be determined using the methods described in Intl. Pat. Appl. Publ. No. WO 2004/056869 and Holst, B.; Cygankiewicz, A.; Halkjaer, T.; Ankersen, A.; Schwartz, T. W. High constitutive signaling of the ghrelin receptor—identification of a potent inverse agonist. Mol. Endocrinol. 2003, 17, 2201-2210. The results for representative compounds of the invention are provided in Table 5.

TABLE 5
Inverse Agonist Activity of Representative
Compounds of the Invention

| Compound | Activity (IC₅₀)* |
|---|---|
| 152 | D |
| 502 | A |
| 503 | B |
| 504 | B |
| 505 | B |
| 506 | D |

*Activity is expressed as follows: A: IC₅₀ = 1-100 nM, B: IC₅₀ = 100-1000 nM, C: IC₅₀ = 1000-10,000 nM, D: IC₅₀ > 10,000 nM.

The foregoing is illustrative of the present invention and is not to be construed as limiting thereof. The invention is defined by the following claims, with equivalents of the claims to be included therein.

What is claimed is:

1. A compound of the formula (I): 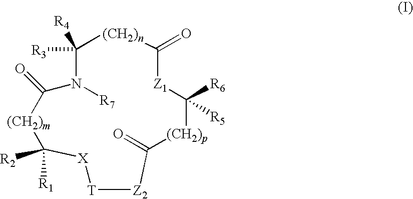
or an optical isomer, enantiomer, diastereomer, racemate or stereochemical mixture thereof, wherein:
X is NR₁₃, wherein R₁₃ is hydrogen, C₁₋₄ alkyl or R₁₂ and R₂ together form a 3-, 4, 5-, 6- or 7-membered heterocyclic ring, wherein the ring optionally comprises an O, S or additional N atom in the ring and is optionally substituted with R₈, wherein R₈ is a substituted cycloalkyl, a substituted fused cycloalkyl, a heterocyclic, a substituted heterocyclic, an aryl, a substituted aryl, a heteroaryl or a substituted heteroaryl ring for hydrogen atoms on two adjacent atoms;
Z₁ is NR₁₁, wherein R₁₁ is hydrogen, C₁₋₄ alkyl or R₁₁ together with R₃ form a 4-, 5-, 6-, 7- or 8-membered heterocyclic ring, wherein the ring optionally comprises an O, S or additional N atom in the ring and is optionally substituted with R₈ as defined previously;
Z₂ is NH;
m, n and p are each 0;
R₁ and R₆ are each independently hydrogen;
R₂ is —(CH₂)ₛCH₃, —CH(CH₃)(C₂)ₜCH₃, —(CH₂)ᵤCH(CH₃)₂, —C(CH₃)₃, —(CH₂)ᵥ—R₁₄, —CH(OR₁₅)CH₃, cycloalkyl, or substituted cycloalkyl, wherein s is 1, 2, 3, 4 or 5; t is 1, 2 or 3; u is 0, 1, 2 or 3; and v is 0, 1, 2, 3 or 4; R₁₄ is aryl, substituted aryl, heteroaryl, substituted heteroaryl, cycloalkyl or substituted cycloalkyl; R₁₅ is hydrogen, alkyl, substituted alkyl, cycloalkyl, substituted cycloalkyl, heterocyclic, substituted heterocyclic, acyl, amino acyl, sulfonyl, carboxyalkyl, carboxyaryl, amido, aryl, substituted aryl, heteroaryl or substituted heteroaryl; or, alternatively, R₂ and R₁₃ together form a 3-, 4-, 5-, 6- or 7-membered heterocyclic ring, wherein the ring optionally comprises an O, S or additional N atom in the ring and is optionally substituted with R % as defined previously;
R₃ and R₄ are each independently hydrogen or an amino acid side chain comprising —CH₃, —CH₂CH₃, —CH(CH₃)₂, —CR₁₇ₐR_{17b}(OR₁₆)—; or alternatively, R₃ and 4 together or R₃ and R₇ together form a 3-, 4-, 5-, 6- or 7-membered ring, respectively, optionally comprising an O or S atom in the ring and optionally substituted with R₈ as defined previously; or alternatively, R₃ and R₁₁ together form a 4-, 5-, 6-, 7- or 8-membered heterocyclic ring, wherein the ring optionally comprises an O, S or additional N atom in the ring and optionally substituted with R₈ as defined previously;
wherein:
R₁₆ is hydrogen, alkyl, substituted alkyl, cycloalkyl, substituted cycloalkyl, heterocyclic, substituted heterocyclic, acyl, amino acyl, sulfonyl, carboxyalkyl, carboxyaryl, amido, aryl, substituted aryl, heteroaryl and substituted heteroaryl; and
R₁₇ₐ and R_{17b} are each independently hydrogen, —CH₃, —CH₂CH₃—CH(CH₃)₂ or —C(CH₃)₃;
R₅ is an amino acid side chain comprising —(CH₂)_{w}CH₃, —CH(CH₃)(CH₂)ₓCH₃, —(CH₂)_{y}CH(CH₃)₂, —C(CH₃)₃, —(CH₂)_{z1}—R₁₈ₐ, —(CR₁₁₀R₁₁₁)_{z2}—R_{18b} wherein w is 2, 3, 4 or 5; x is 1, 2 or 3; y is 0, 1, 2 or 3; z1 is 0, 1, 2, 3 or 4; z2 is 0, 1 or 2; R₁₈ₐ and R_{18b} are each independently aryl, substituted aryl, heteroaryl, substituted heteroaryl, cycloalkyl and substituted cycloalkyl; R₁₁₀ and R₁₁₁ are each independently hydrogen C₁-C₄ alkyl, hydroxyl, amino or fluoro, with the proviso that at least one of R₁₁₀ and R₁₁₁ is not hydrogen;
R₇ is hydrogen, C₁-C₄ alkyl or R₇ and R₃ together form a 3-, 4-, 5-, 6- or 7-membered ring, respectively, optionally comprising an O or S atom in the ring and optionally substituted with R₈ as defined below;
R₈ is substituted for one or more hydrogen atoms on the 3-, 4-, 5-, 6- or 7-membered ring structure and is independently selected from the group consisting of alkyl, substituted alkyl, cycloalkyl, substituted cycloalkyl, heterocyclic, substituted heterocyclic, aryl, substituted aryl, heteroaryl, substituted heteroaryl, hydroxy, alkoxy, aryloxy, oxo, amino, halogen, formyl, acyl, carboxy, carboxyalkyl, carboxyaryl, amido, carbamoyl, guanidino, ureido, amidino, mercapto, sulfinyl, sulfonyl and sulfonamide, or, alternatively, R₈ is a fused cycloalkyl, a substituted fused cycloalkyl, a fused heterocyclic, a substituted fused heterocyclic group, a fused aryl, a substituted fused aryl, a fused heteroaryl or a substituted fused heteroaryl ring substituted for hydrogen atoms on two adjacent atoms; and
T is a bivalent radical of formula IV:
-U-(CH₂)_{d}-W-Y-Z-(CH₂)ₑ— (IV)
wherein d and e are each independently 0, 1, 2, 3, 4 or 5; Y and Z are each optionally present; U is —CR₂₁R₂₂—, or —C(═O)— and is bonded to X of formula I; W, Y and Z are each —O—, —NR₂₃—, —S—, —SO—, —SO₂—, —C(═O)—O—, —O—C(═O)—, —C(═O)—NH—; —NH—C(═O)—, —SO₂—NH—, —NH—SO₂—, —CR₂₄R₂₅—, —CH═CH— with the configuration Z or E, —C≡C—, or the ring structures below: 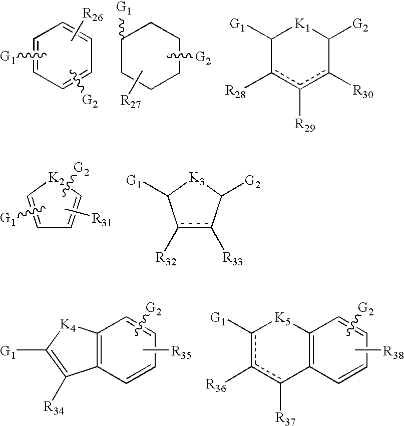
wherein G₁ and G₂ are each independently a covalent bond or a bivalent radical selected from the group consisting of —O—, —NR₃₉—, —S—, —SO—, —SO₂—, —C(═O)—, —C(═O)—O—, —O—C(═O)—, —C(═O)NH—, —NH—C(═O)—, —SO₂—NH—, —NH—SO₂—, —CR₄₀R₄₁—, —CH═CH— with the configuration Z or E, and —C≡C—; with G₁ being bonded closest to the group U; wherein any carbon atom in the rings not otherwise defined, is optionally replaced by N, with the proviso that the ring cannot contain more than four N atoms; K₁, K₂, K₃, K₄ and K₅ are each independently O, NR₄₂ or S, wherein R₄₂ is defined below;
R₂₁ and R₂₂ are each independently hydrogen, lower alkyl, or substituted lower alkyl, or R₂₁ and R₂₂ together form a 3- to 12-membered cyclic ring optionally comprising one or more heteroatoms selected from the group consisting of O, S and N, wherein the ring is optionally substituted with R₈ as defined previously;
R₂₃, R₃₉ and R₄₂ are each independently hydrogen, alkyl, substituted alkyl, cycloalkyl, substituted cycloalkyl, heterocyclic, substituted heterocyclic, aryl, substituted aryl, heteroaryl, substituted heteroaryl, formyl, acyl, carboxyalkyl, carboxyaryl, amido, amidino, sulfonyl or sulfonamido;
R₂₄ and R₂₅ are each independently hydrogen, lower alkyl, substituted lower alkyl, R_{AA}, wherein R_{AA} is a side chain of a typical or unusual amino acid, or R₂₄ and R₂₅ together form a 3- to 12-membered cyclic ring optionally comprising one or more heteroatoms selected from the group consisting of O, S and N; or one of R₂₄ or R₂₅ is hydroxy, alkoxy, aryloxy, amino, mercapto, carbamoyl, amidino, ureido or guanidino while the other is hydrogen, lower alkyl or substituted lower alkyl, except when the carbon to which R₂₄ and R₂₅ are bonded is also bonded to another heteroatom;
R₂₆ is optionally present and, when present, is substituted for one or more hydrogen atoms on the indicated ring and each is independently selected from the group consisting of halogen, trifluoromethyl, alkyl, substituted alkyl, cycloalkyl, substituted cycloalkyl, heterocyclic, substituted heterocyclic, aryl, substituted aryl, heteroaryl, substituted heteroaryl, hydroxy, alkoxy, aryloxy, amino, formyl, acyl, carboxy, carboxyalkyl, carboxyaryl, amido, carbamoyl, guanidino, ureido, amidino, cyano, nitro, mercapto, sulfinyl, sulfonyl and sulfonamido;
R₂₇ is optionally present and, when present, is substituted for one or more hydrogen atoms on the indicated ring and each is independently selected from the group consisting of alkyl, substituted alkyl, cycloalkyl, substituted cycloalkyl, heterocyclic, substituted heterocyclic, aryl, substituted aryl, heteroaryl, substituted heteroaryl, hydroxy, alkoxy, aryloxy, oxo, amino, formyl, acyl, carboxy, carboxyalkyl, carboxyaryl, amido, carbamoyl, guanidino, ureido, amidino, mercapto, sulfinyl, sulfonyl and sulfonamido;
R₂₈, R₂₉, R₃₀, R₃₂, R₃₃, R₃₄, R₃₆ and R₃₇ are each optionally present and when no double bond is present to the carbon atom to which it is bonded in the ring, two groups are optionally present, and, when present, is substituted for one hydrogen present in the ring, or when no double bond is present to the carbon atom to which it is bonded in the ring, is substituted for one or both of the two hydrogen atoms present on the ring and each is independently selected from the group consisting of alkyl, substituted alkyl, cycloalkyl, substituted cycloalkyl, heterocyclic, substituted heterocyclic, aryl, substituted aryl, heteroaryl, substituted heteroaryl, hydroxy, alkoxy, aryloxy, oxo, amino, formyl, acyl, carboxy, carboxyalkyl, carboxyaryl, amido, carbamoyl, guanidino, ureido, amidino, mercapto, sulfinyl, sulfonyl, sulfonamide and, only if a double bond is present to the carbon atom to which it is bonded, halogen;
R₃₁, R₃₅ and R₃₈ are each optionally present and, when present, are substituted for one or more hydrogen atoms on the indicated ring and each is independently selected from the group consisting of halogen, trifluoromethyl, alkyl, substituted alkyl, cycloalkyl, substituted cycloalkyl, heterocyclic, substituted heterocyclic, aryl, substituted aryl, heteroaryl, substituted heteroaryl, hydroxy, alkoxy, aryloxy, amino, formyl, acyl, carboxy, carboxyalkyl, carboxyaryl, amido, carbamoyl, guanidino, ureido, amidino, cyano, nitro, mercapto, sulfinyl, sulfonyl and sulfonamido; and
R₄₀ and R₄₁ are each independently hydrogen, lower alkyl, substituted lower alkyl, R_{AA} as defined above, or R₄₀ and R₄₁ together form a 3- to 12-membered cyclic ring optionally comprising one or more heteroatoms selected from the group consisting of O, S and N wherein the ring is optionally substituted with R₈ as defined previously, or one of R₄₀ and R₄₁ is hydroxy, alkoxy, aryloxy, amino, mercapto, carbamoyl, amidino, ureido or guanidino, while the other is hydrogen, lower alkyl or substituted lower alkyl, except when the carbon to which R₄₀ and R₄₁ are bonded is also bonded to another heteroatom;
with the proviso that T is not an amino acid residue, dipeptide fragment, tripeptide fragment or higher order peptide fragment comprising standard amino acids;

2. The compound of claim 1, wherein T is selected from one of the following structures: 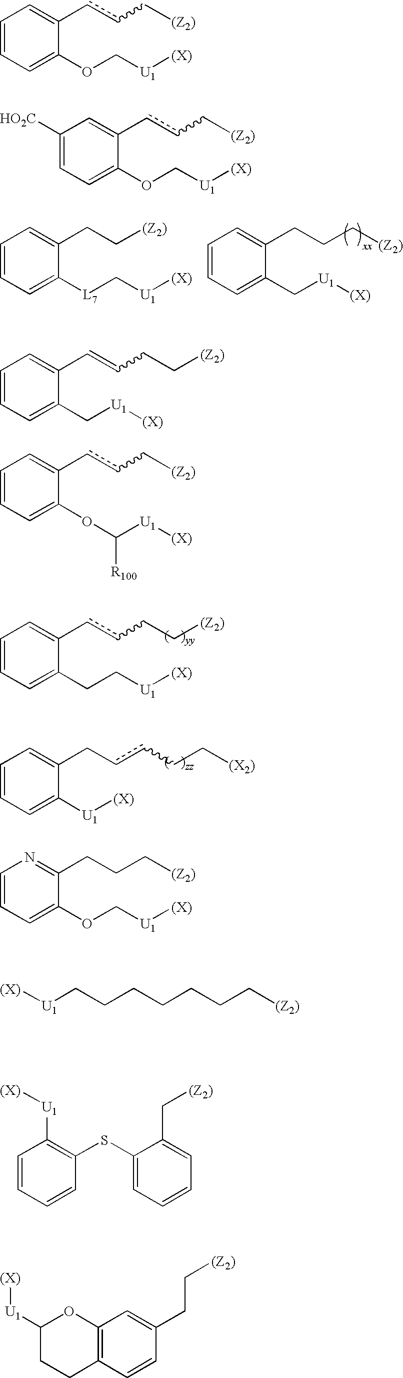 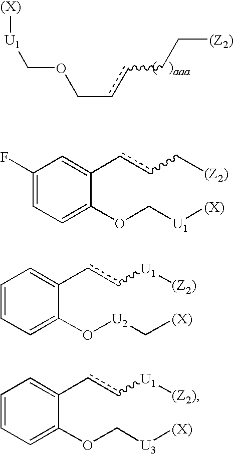
wherein (Z₂) is the site of a covalent bond of T to Z₂, and Z₂ is defined above, and wherein (X) is the site of a covalent bond of T to X, and X is defined above;
U₁ and U₃ are each independently —CR₁₀₁R₁₀₂— or —(═O)—;
U₂ is —CR₁₀₁R₁₀₂—
R₁₀₀ is lower alkyl;
R₁₀₁ and R₁₀₂ are each independently hydrogen, lower alkyl or substituted lower alkyl;
L₇ is —CH₂— or —O—;
xx is 2 or 3;
yy is 1 or 2;
zz is 1 or 2; and
aaa is 0 or 1.

3. The compound of claim 1, wherein:
R₃ is H;
R₄ is —CR₄₃ₐR_{43b}(OR₄₄) where R₄₃ₐ and R_{43b} are each independently hydrogen or lower alkyl and R₄₄ is hydrogen, alkyl, substituted alkyl, aryl, substituted aryl, or acyl; and
R₇ is hydrogen or lower alkyl.

4. A compound of one of the following structures: 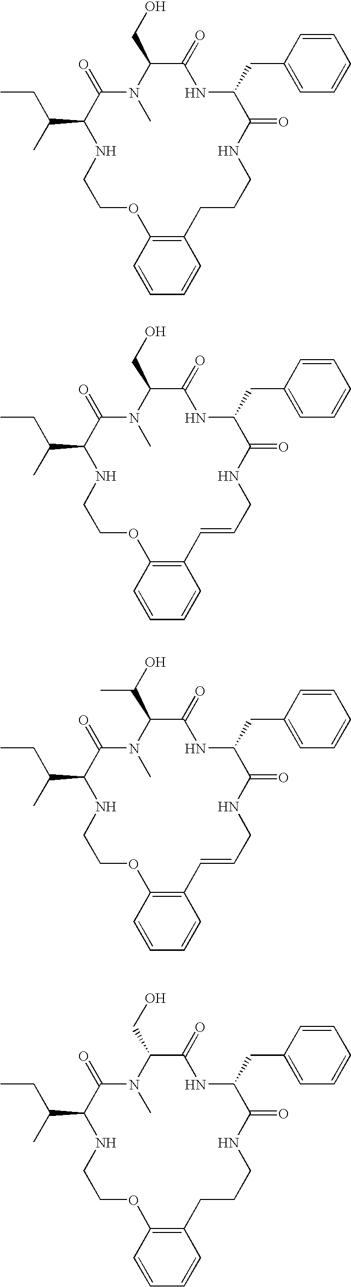 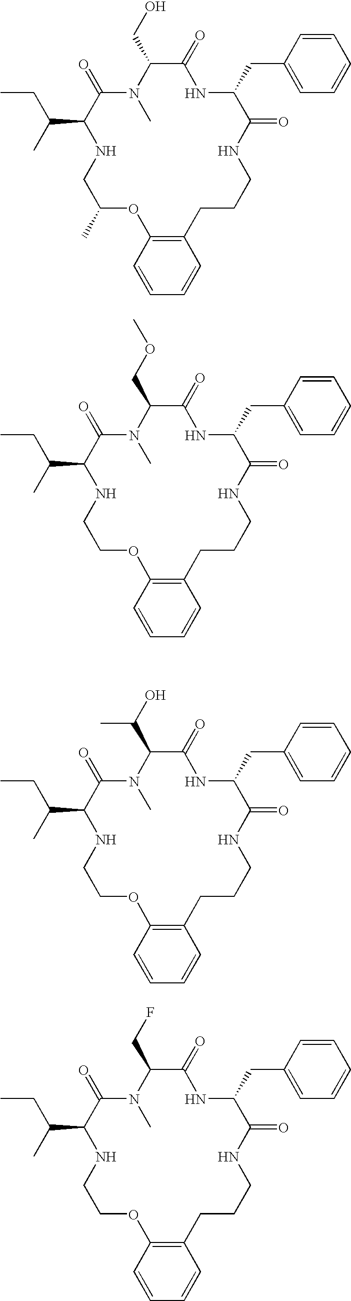 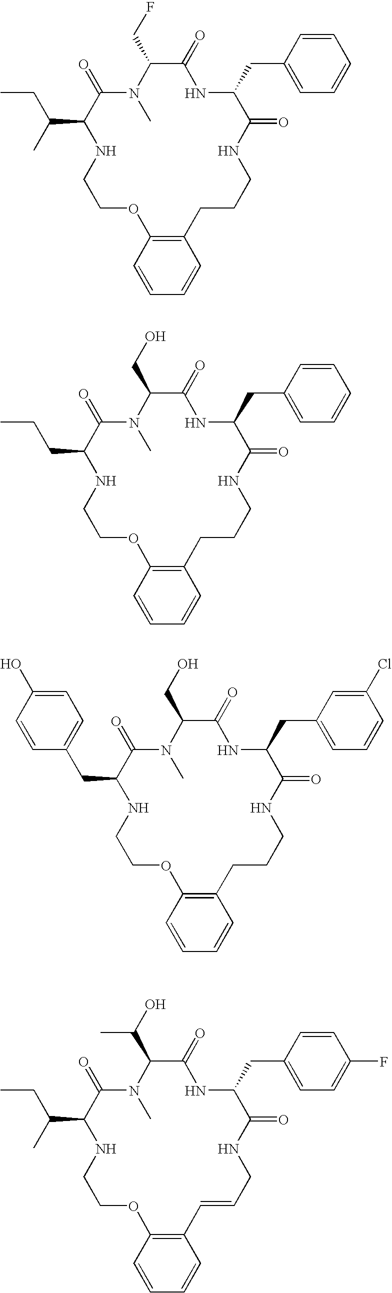 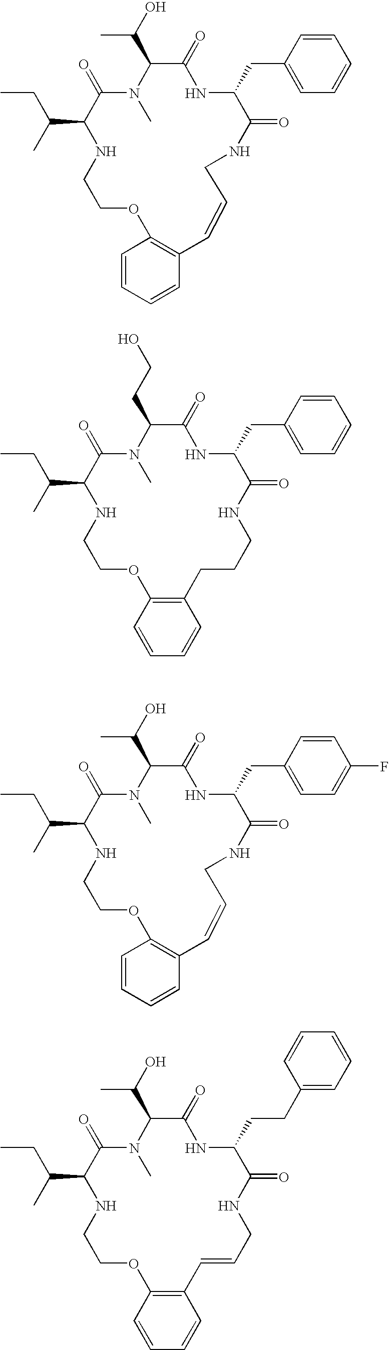 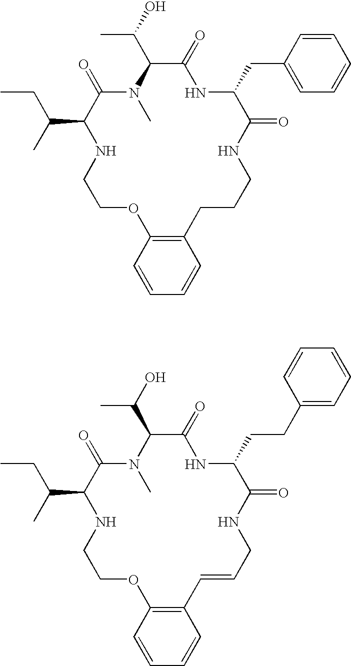 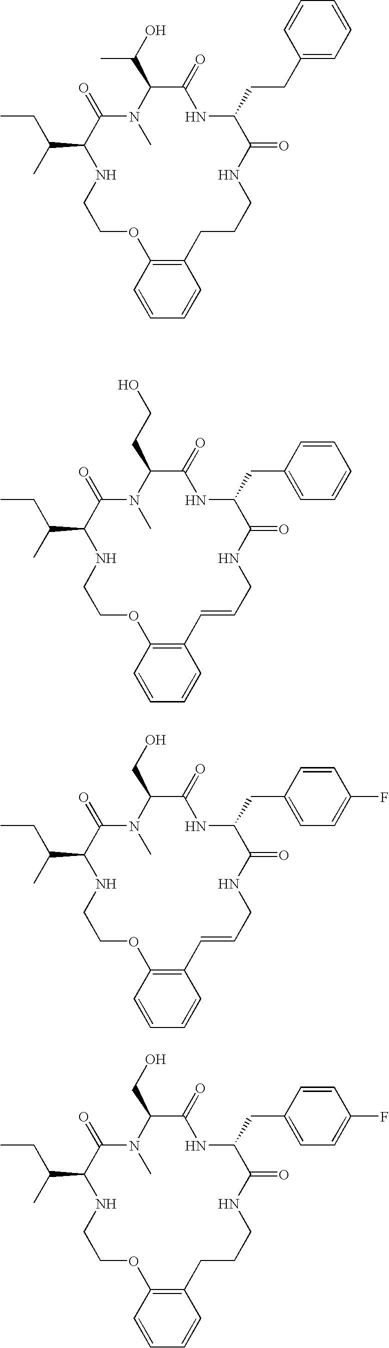 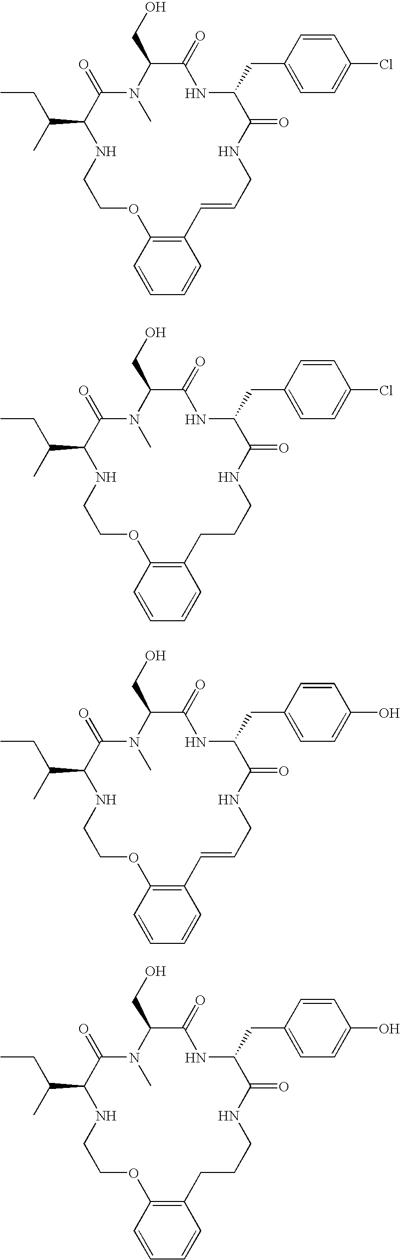 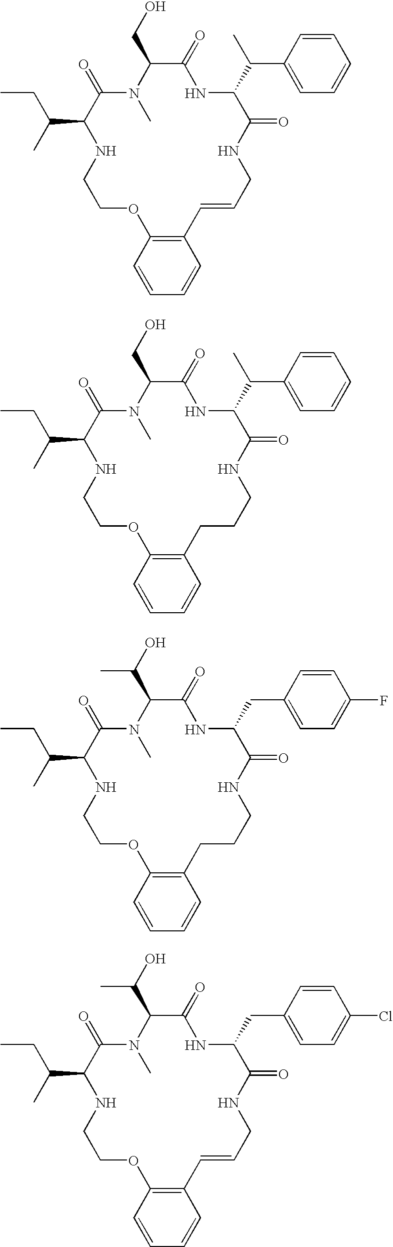 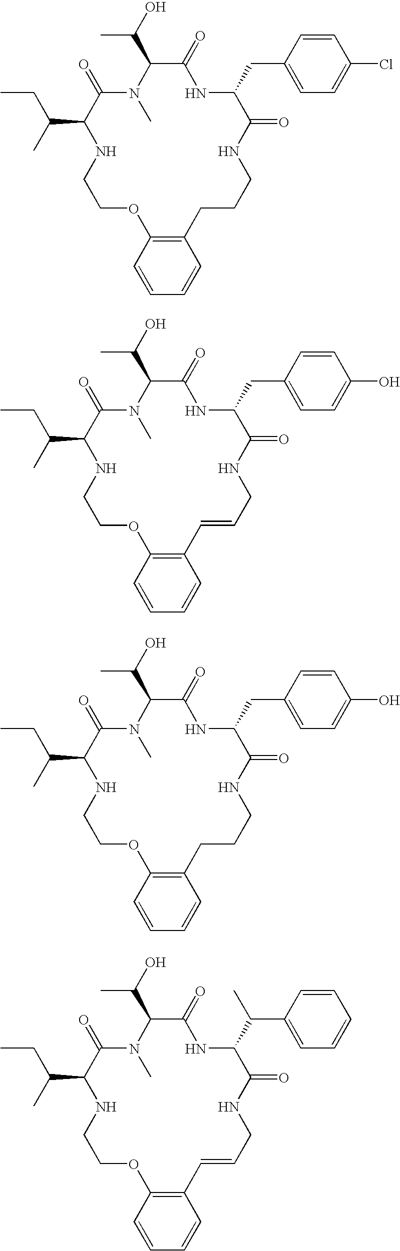 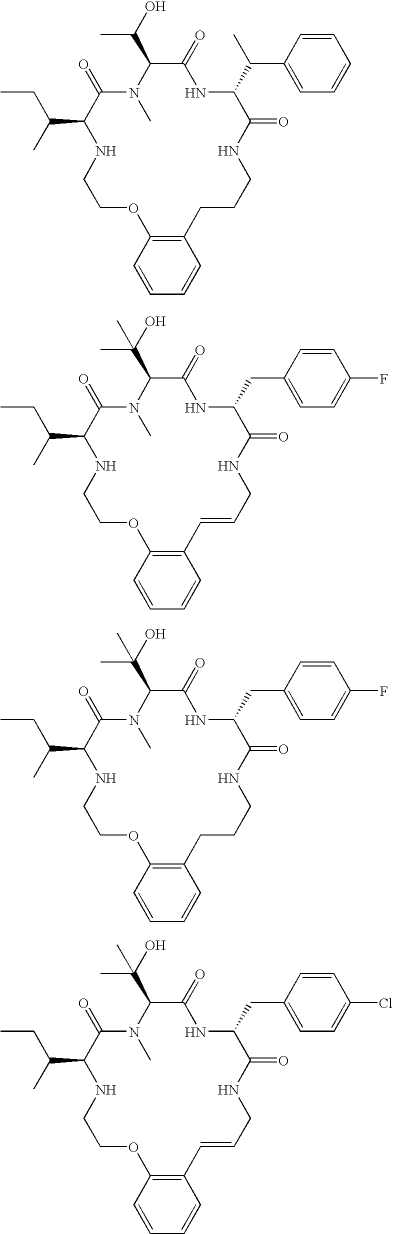 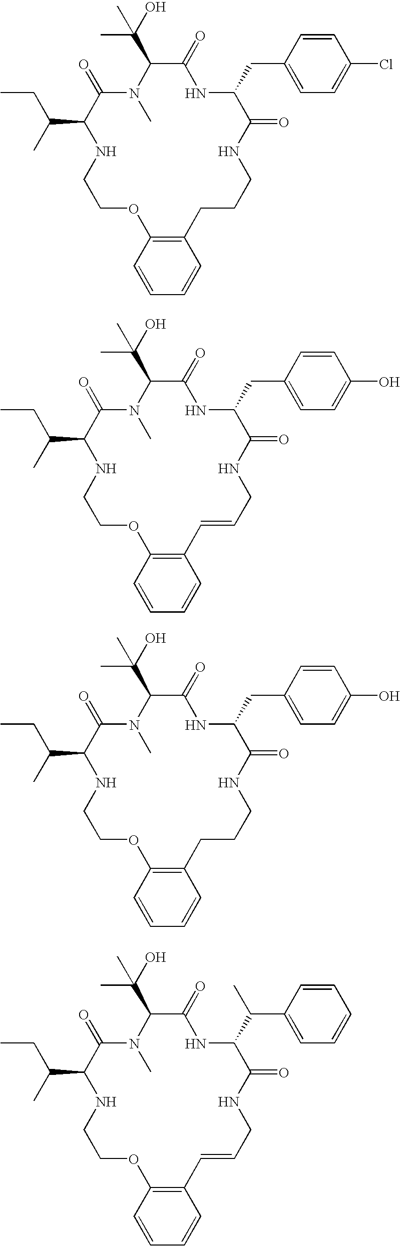 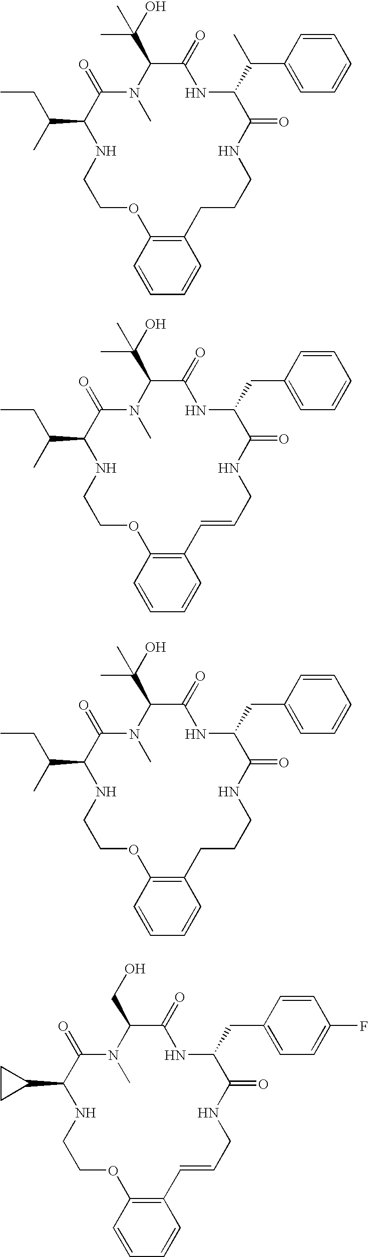 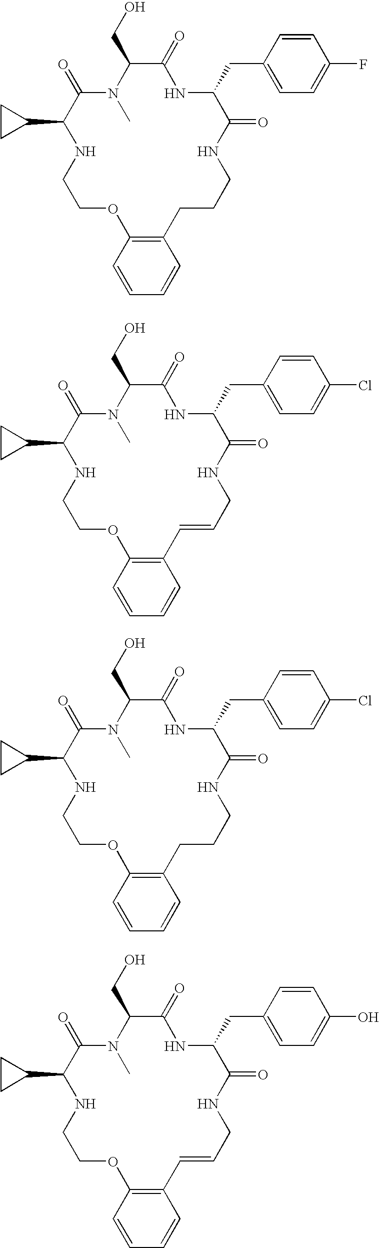 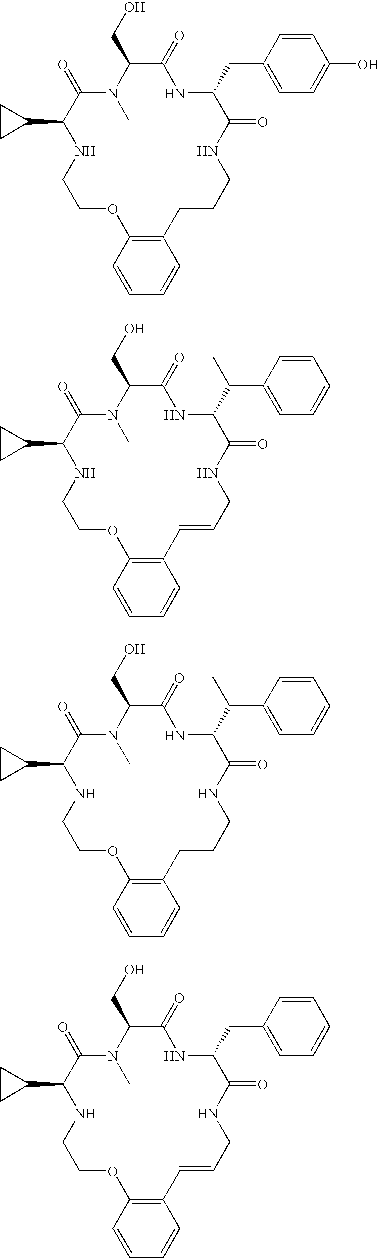 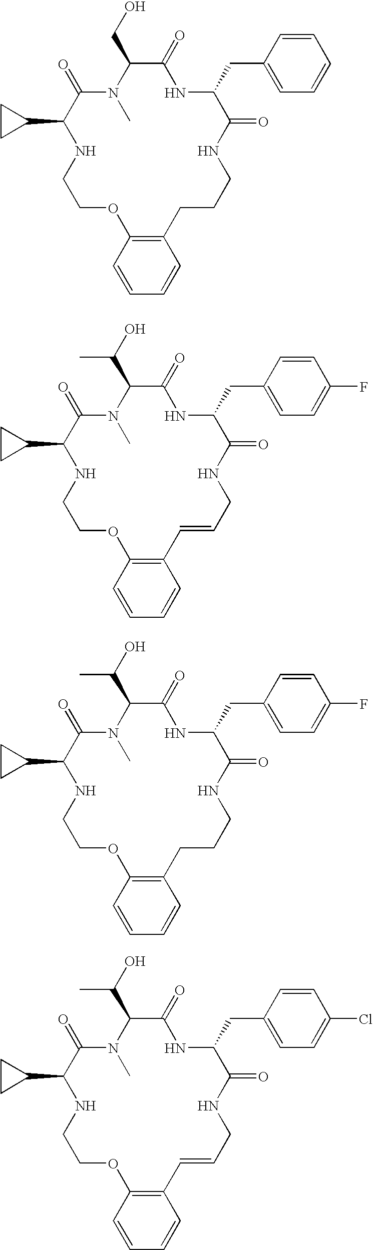 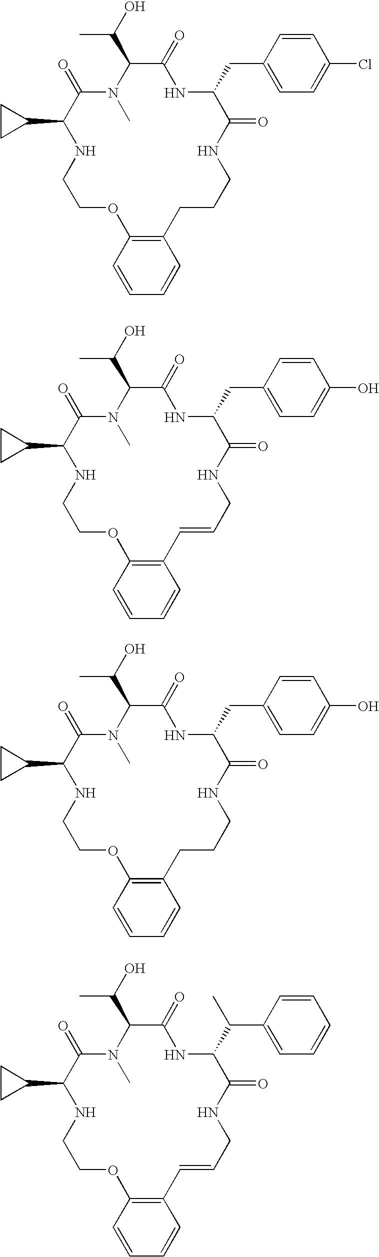 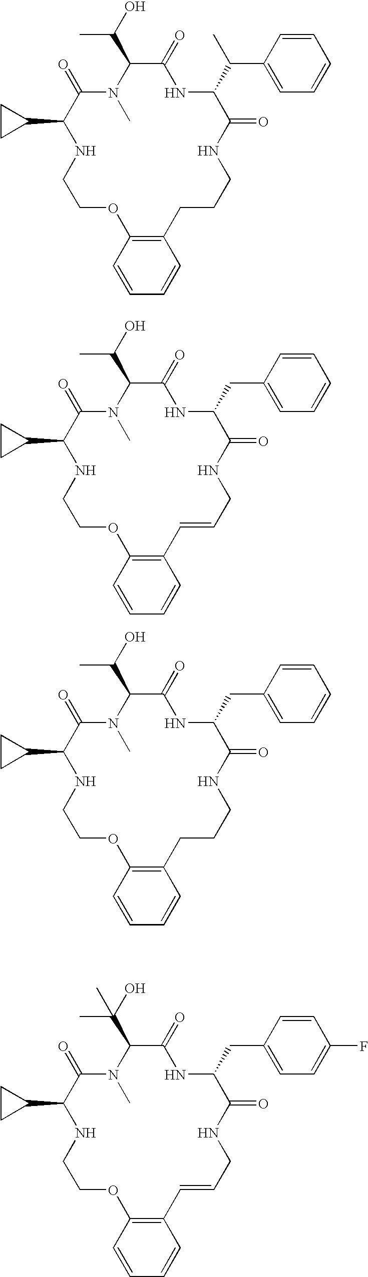 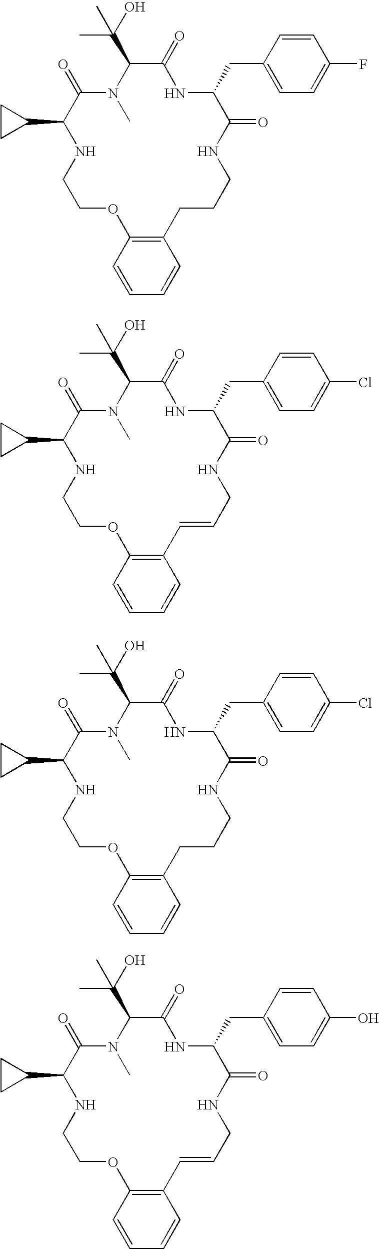 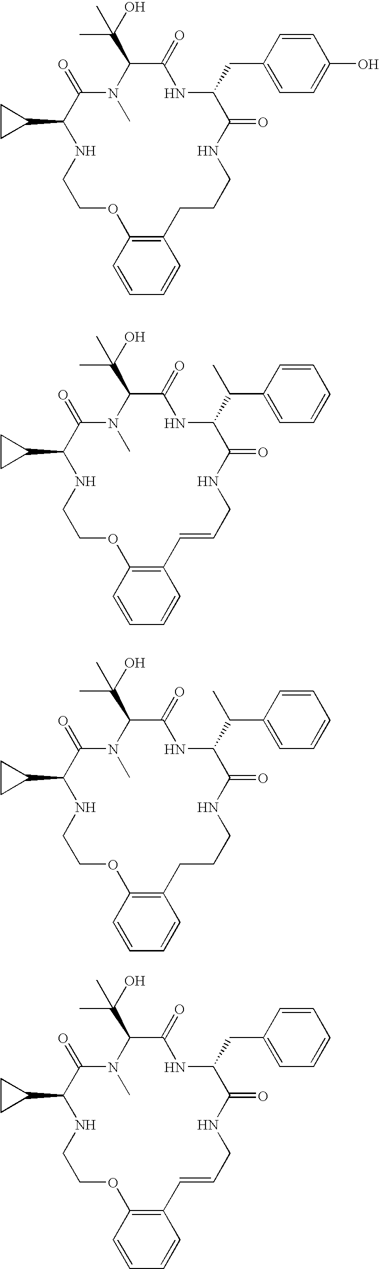 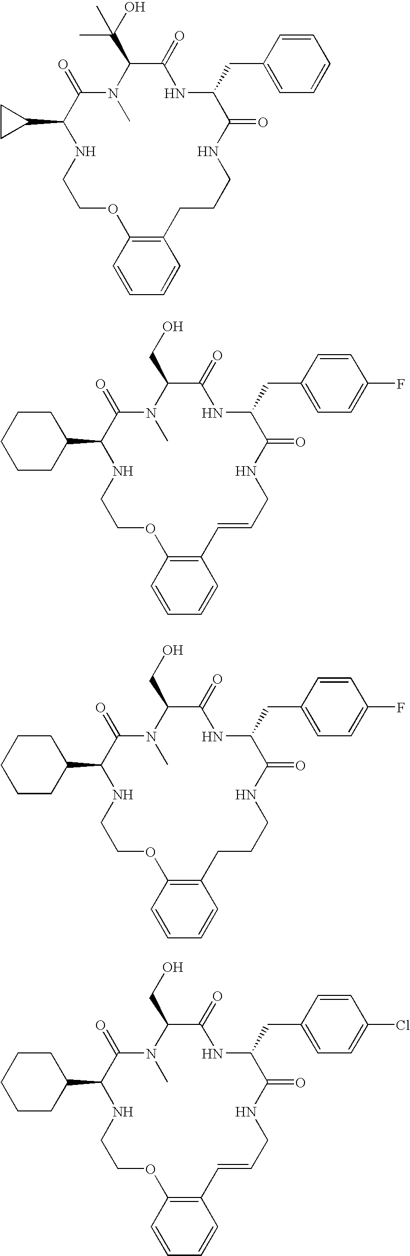 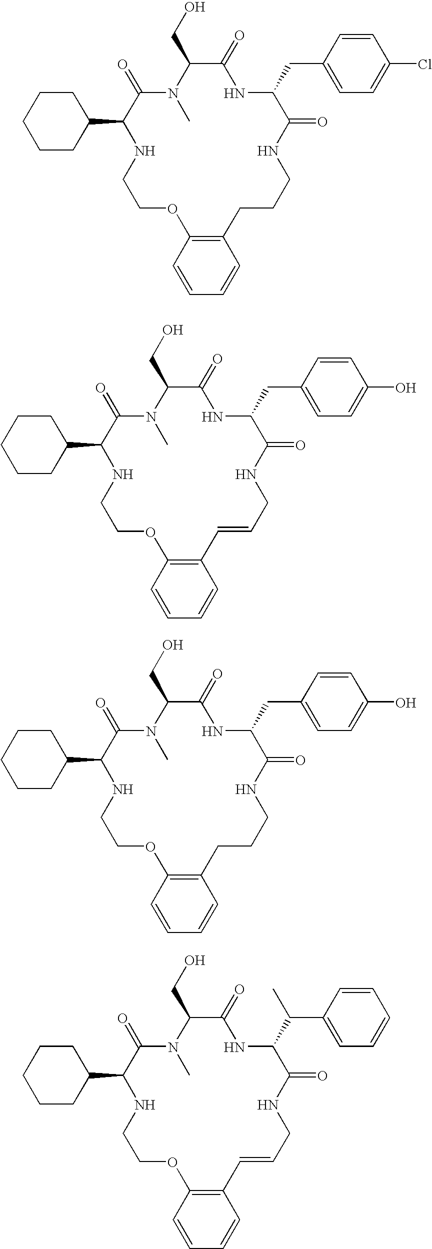 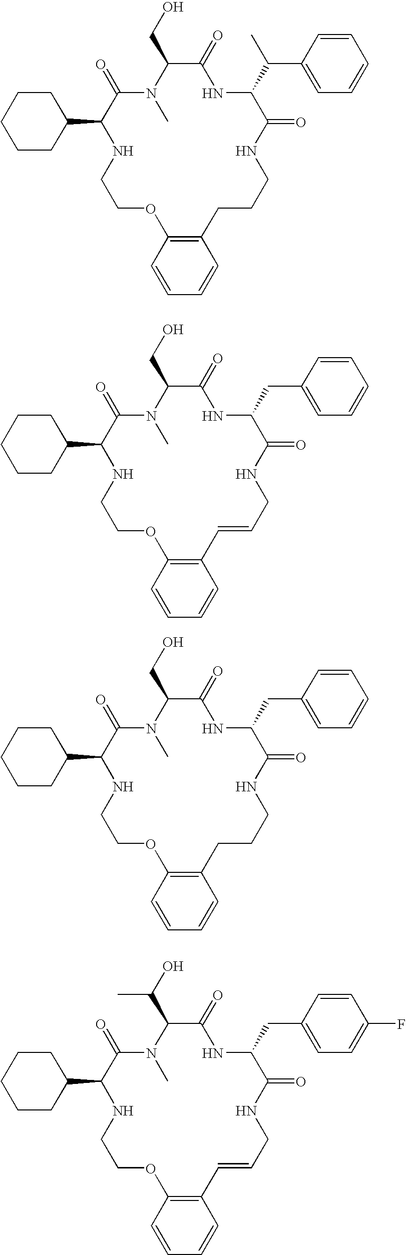 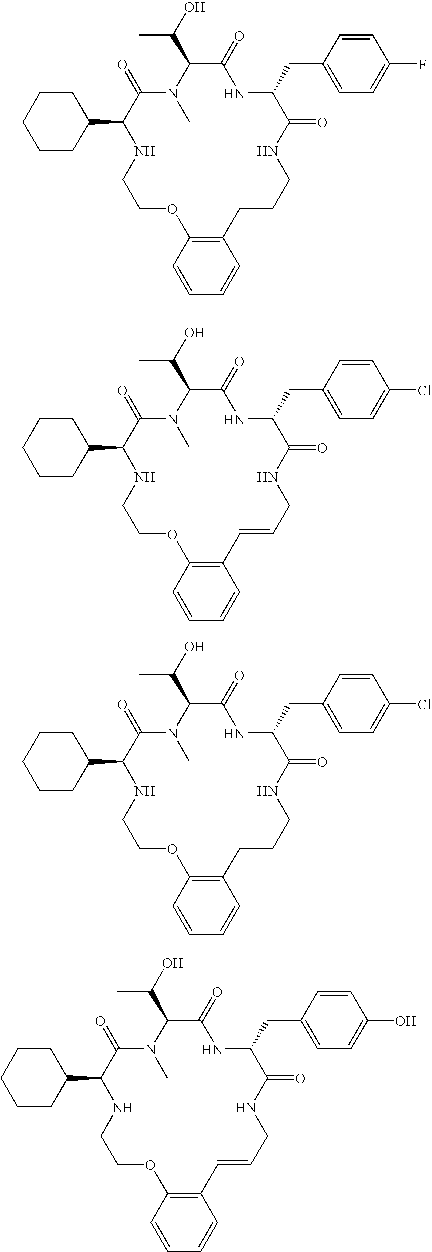 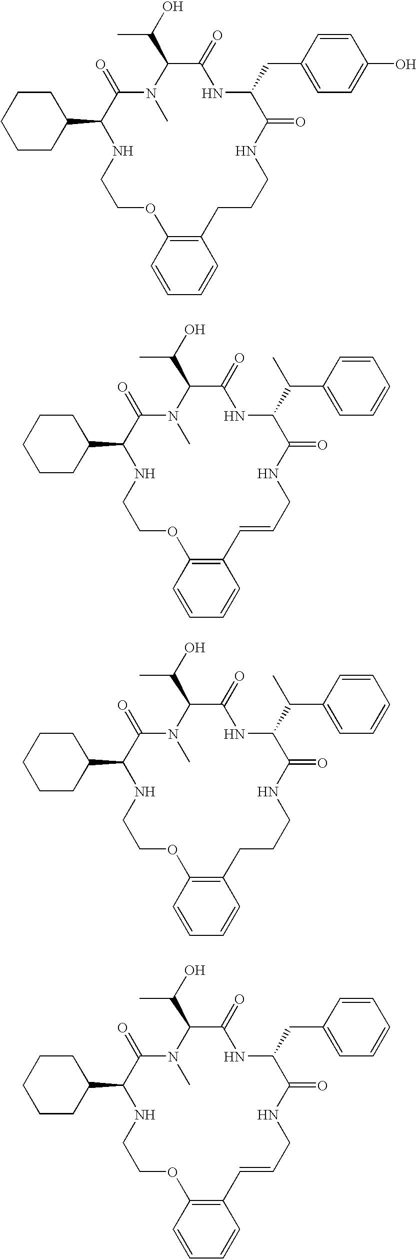 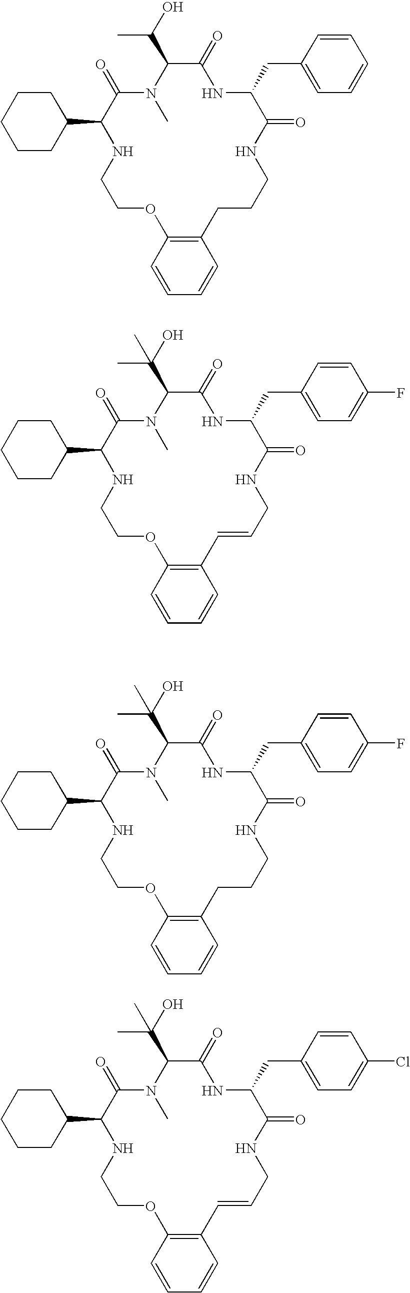 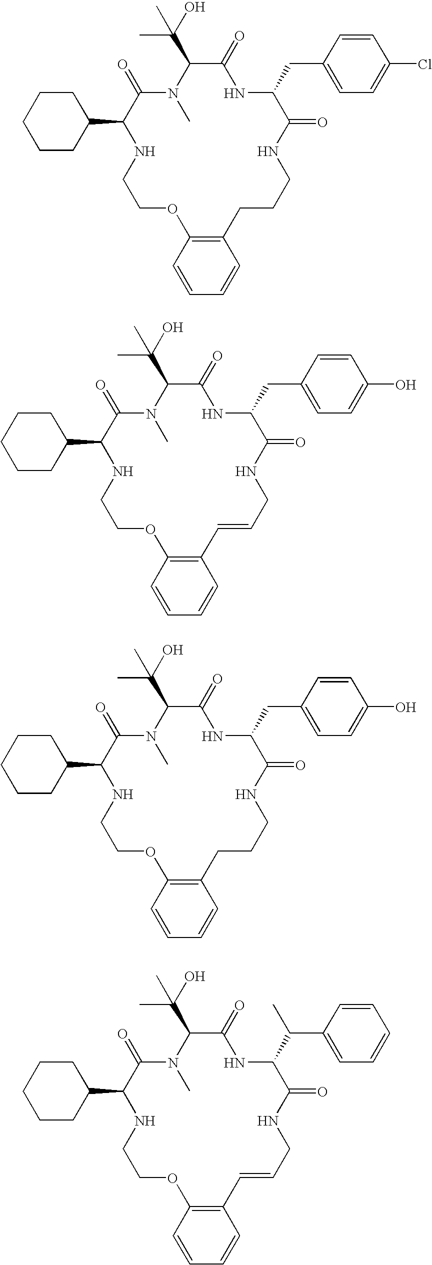 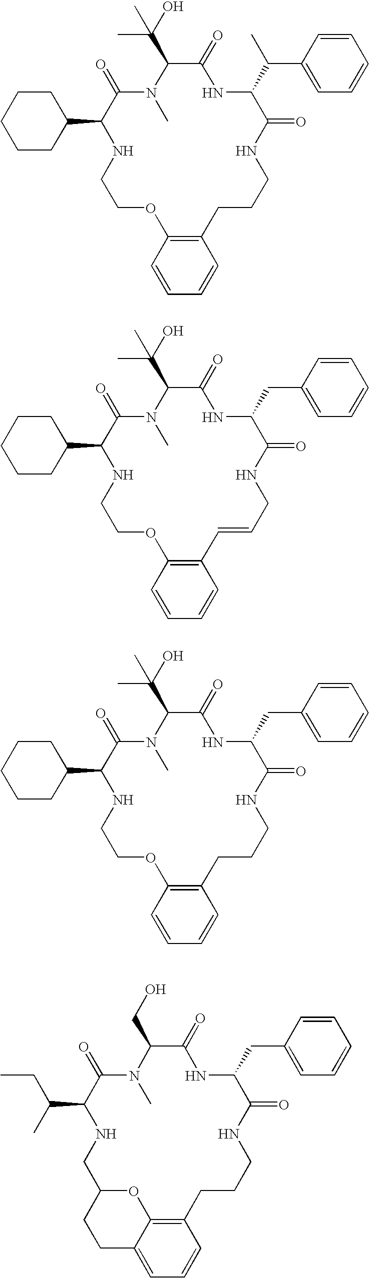 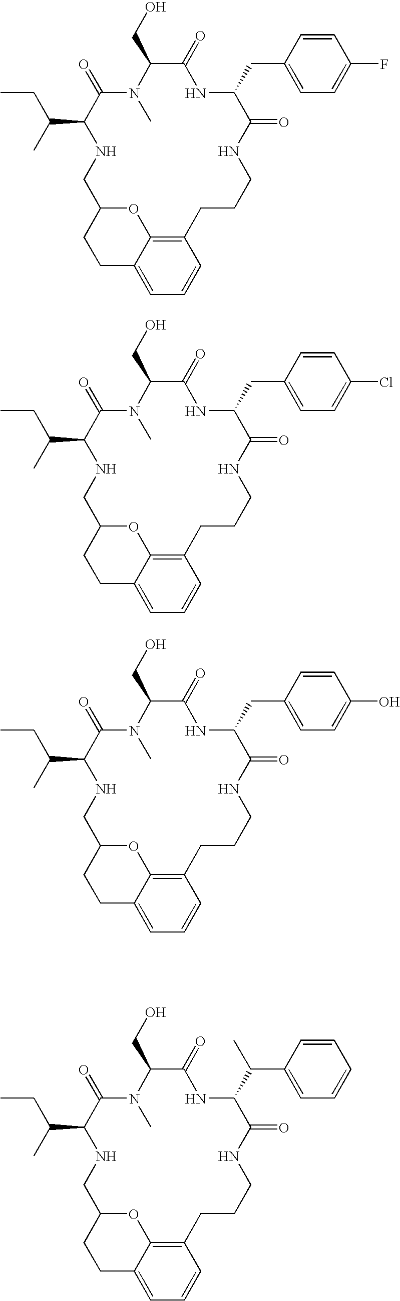 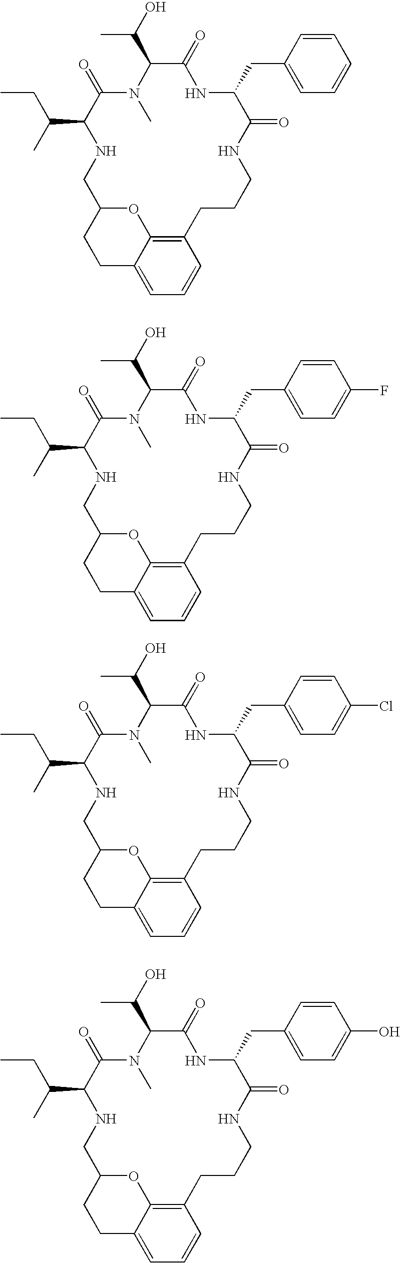 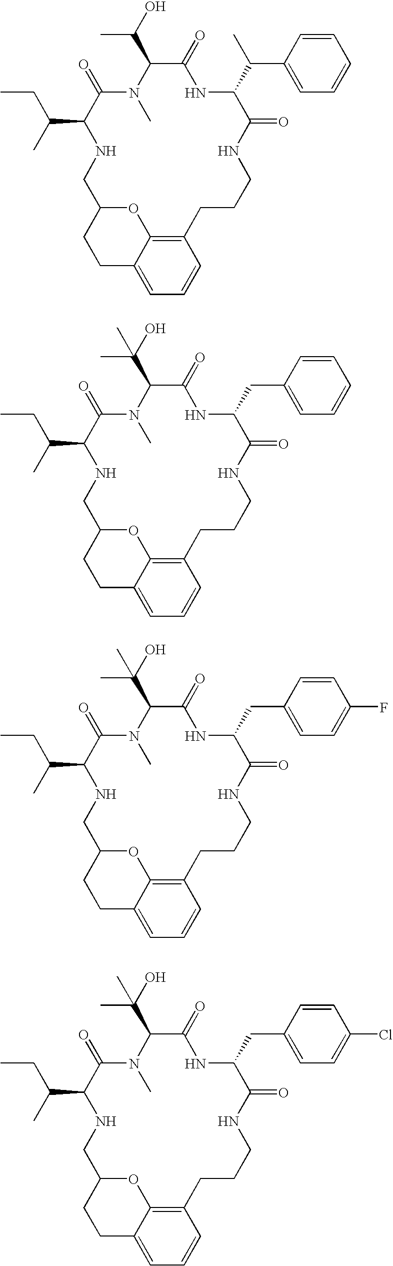 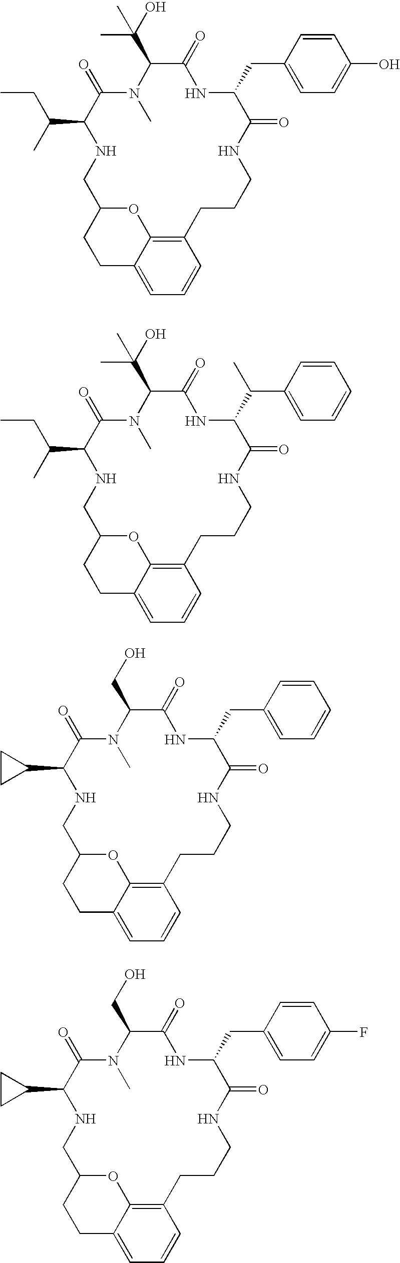 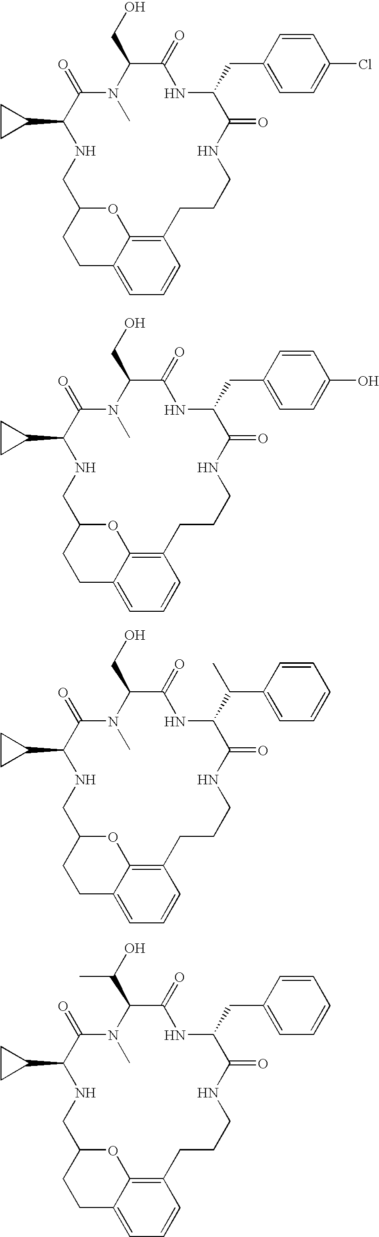 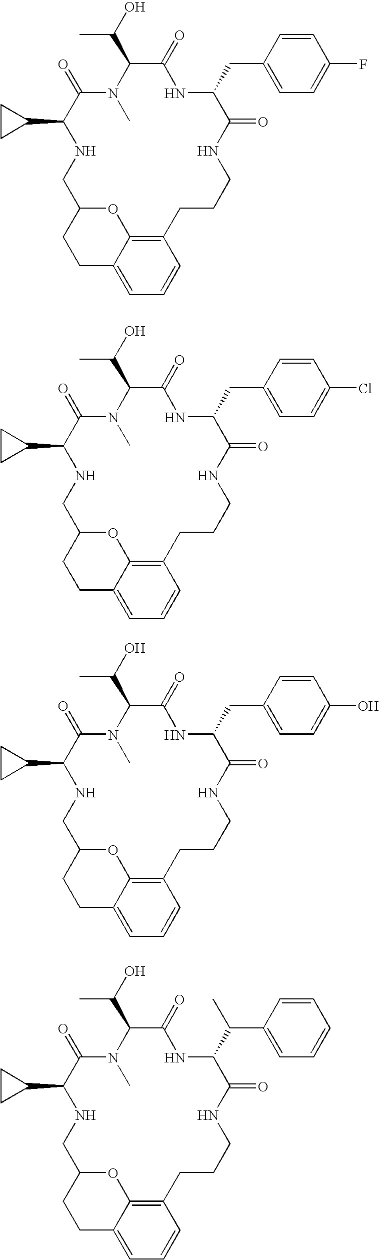 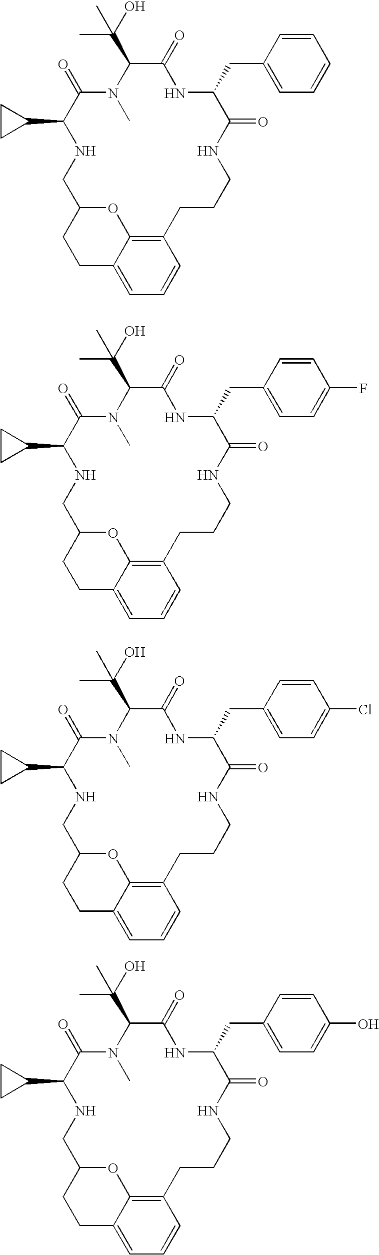 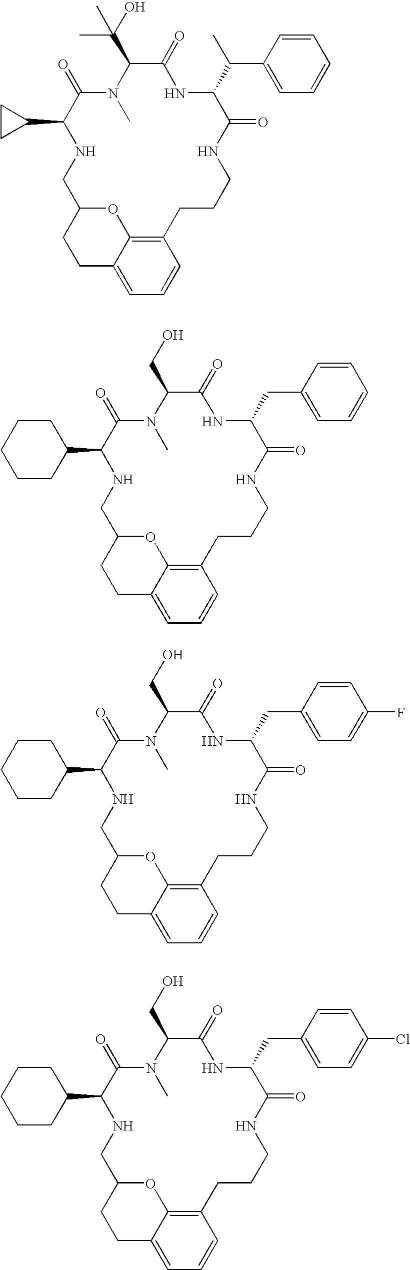 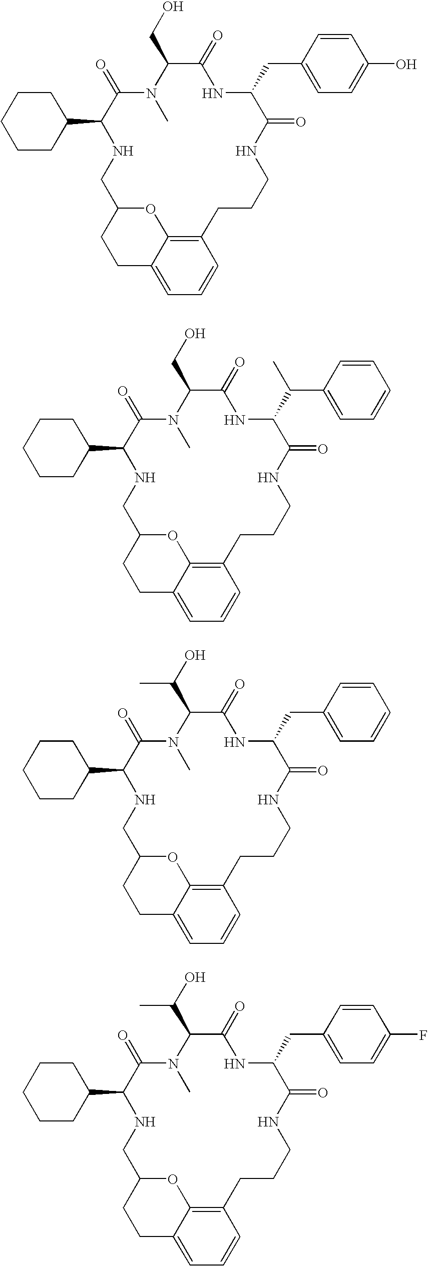 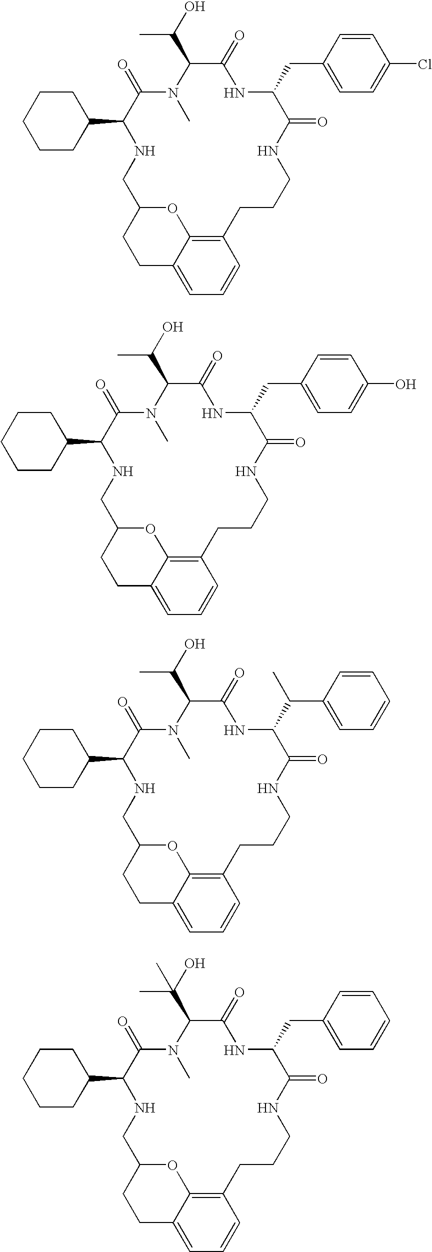 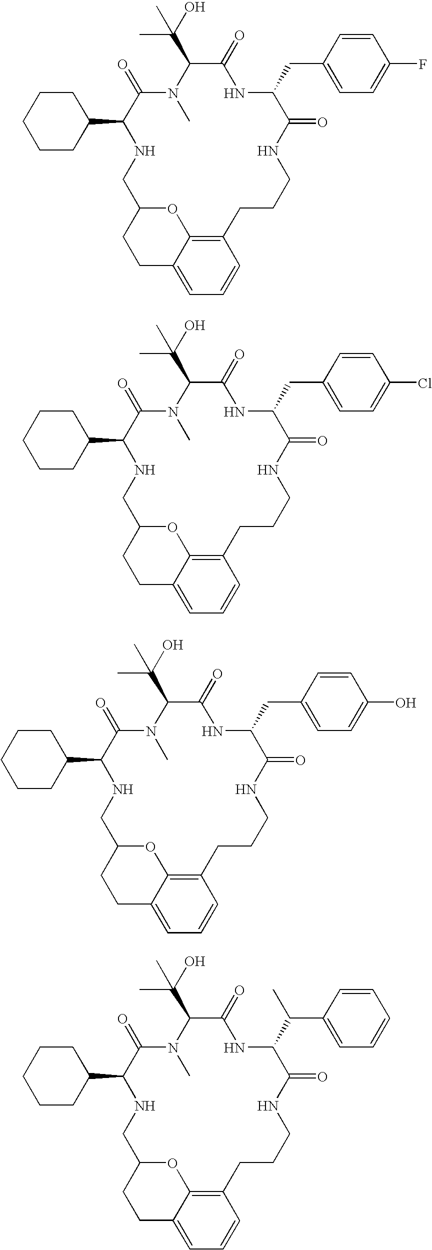
or
an optical isomer, enantiomer, diastereomer, racemate or stereochemical mixture thereof.

5. A pharmaceutical composition comprising:
(a) a compound of claim 1; and
(b) a pharmaceutically acceptable carrier, excipient or diluent.

6. A pharmaceutical composition comprising:
(a) a compound of claim 4; and
(b) a pharmaceutically acceptable carrier, excipient or diluent.

7. A kit comprising one or more containers comprising pharmaceutical dosage units further comprising an effective amount of one or more compounds having the following structure: 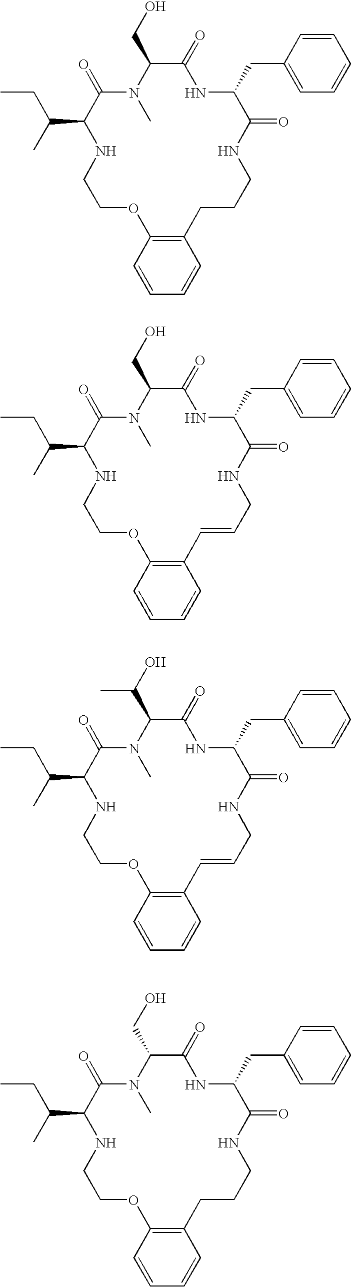 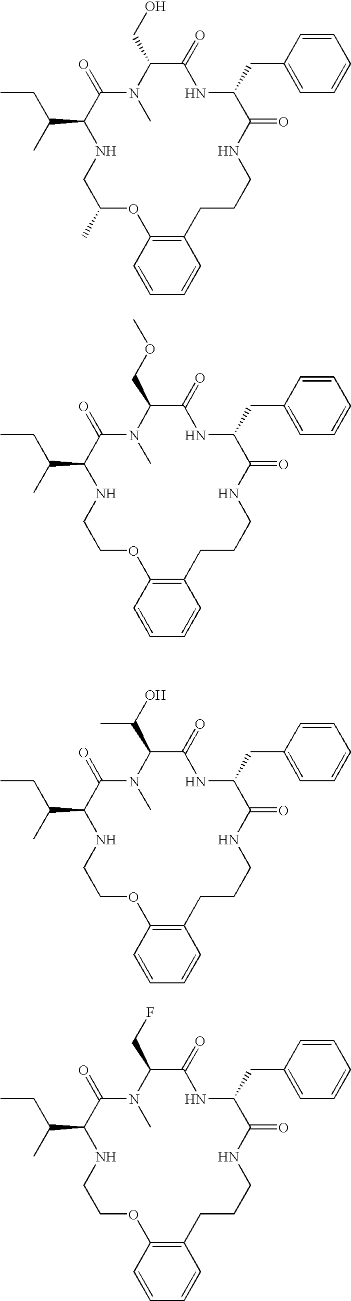 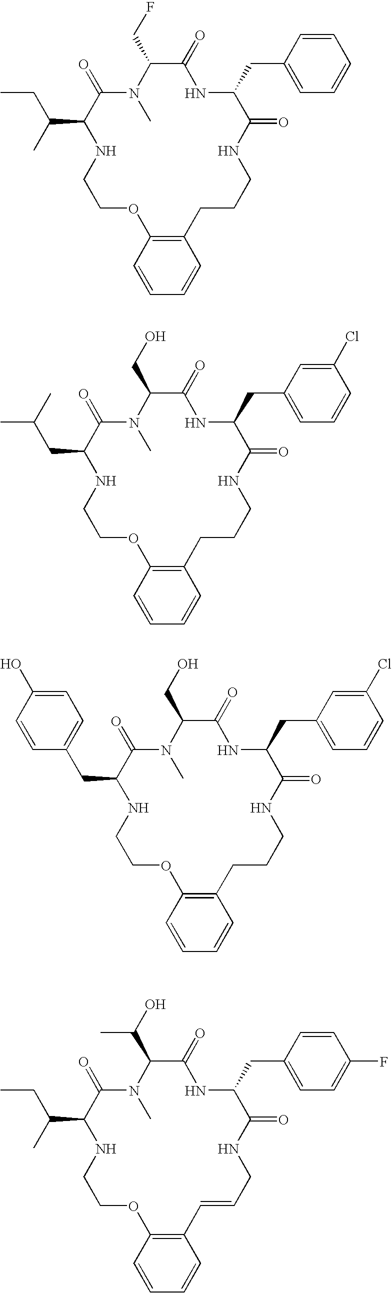 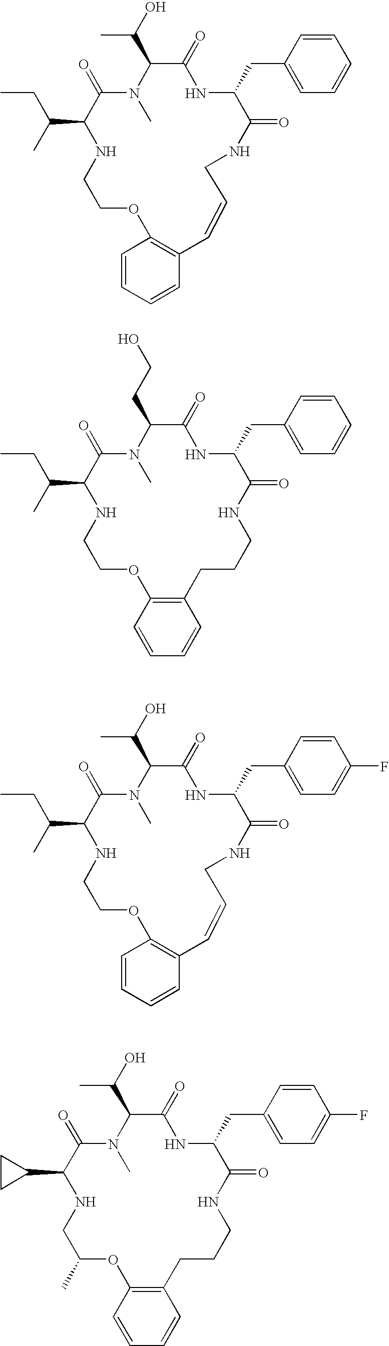 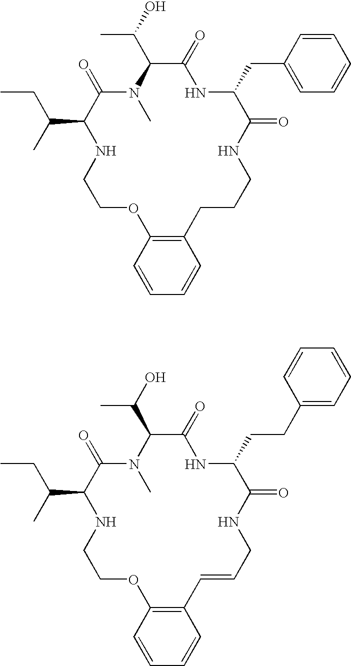 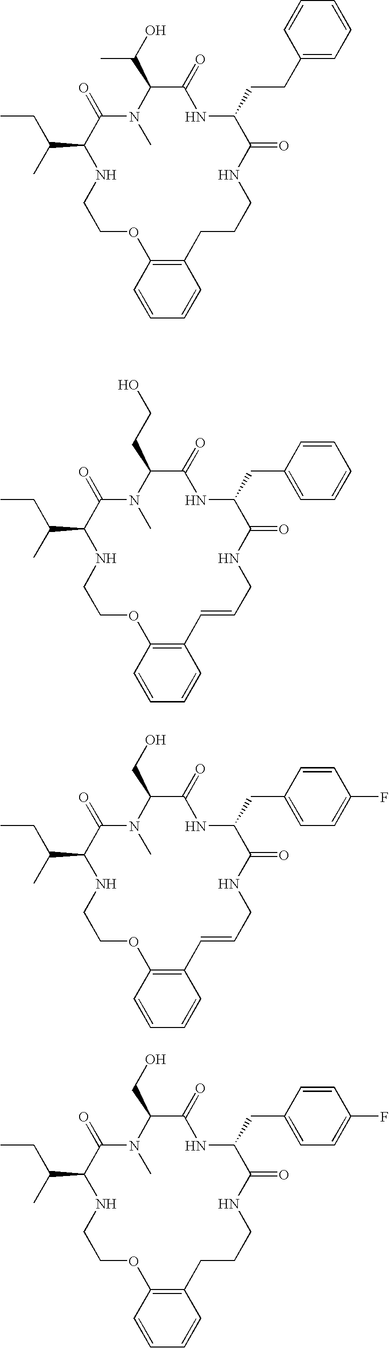 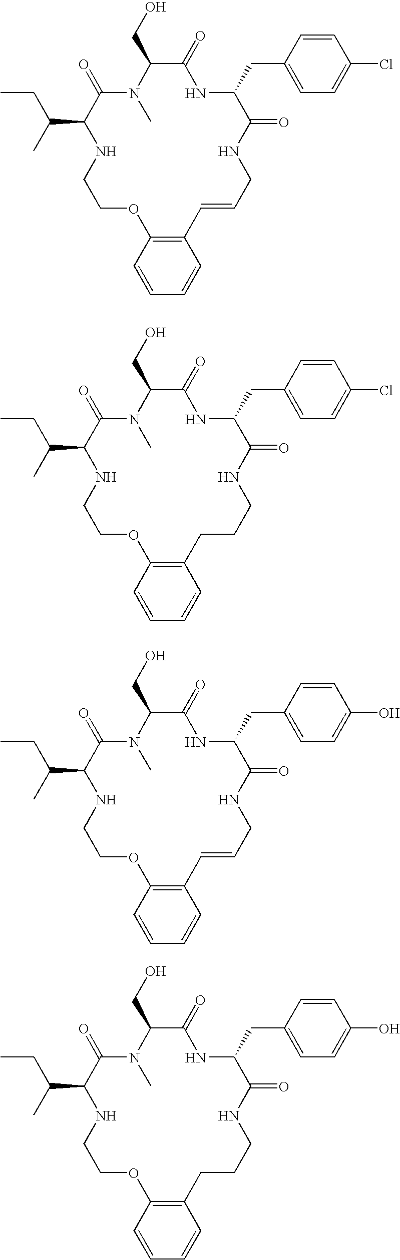 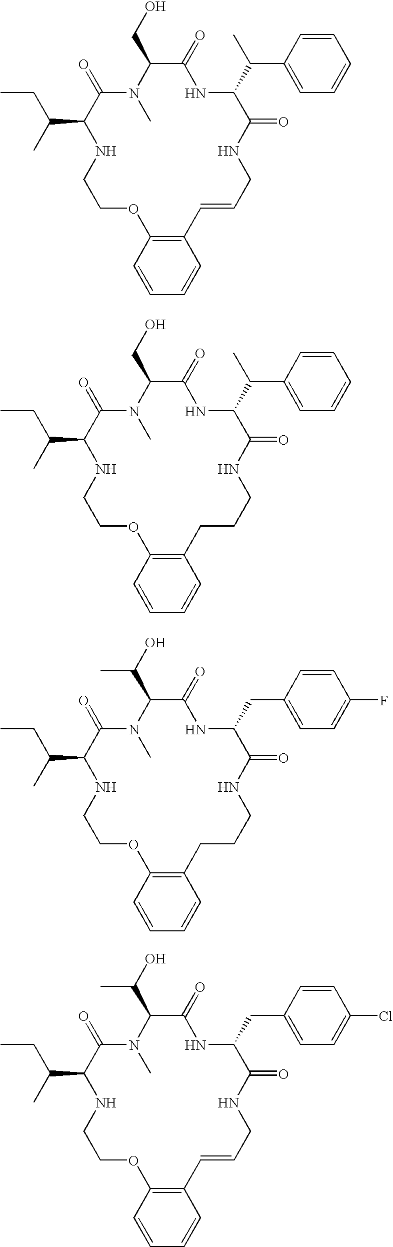 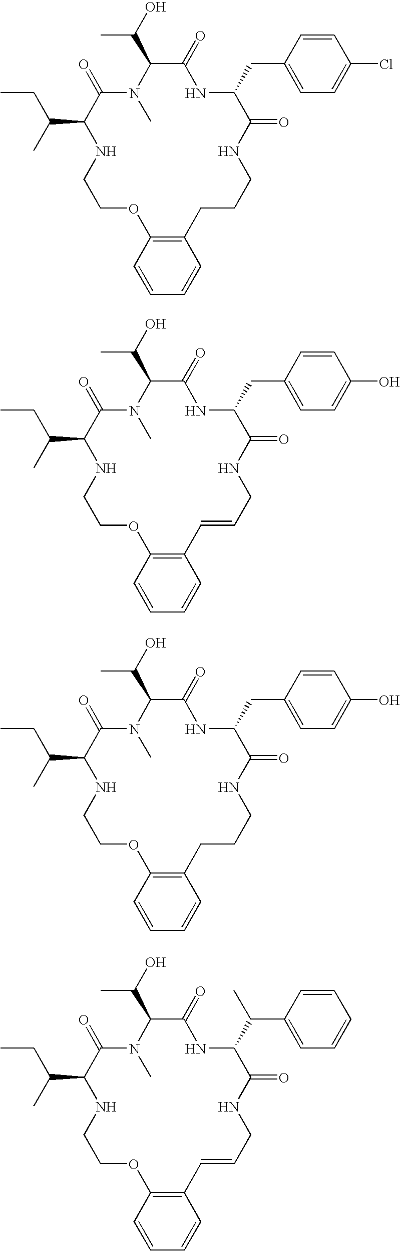 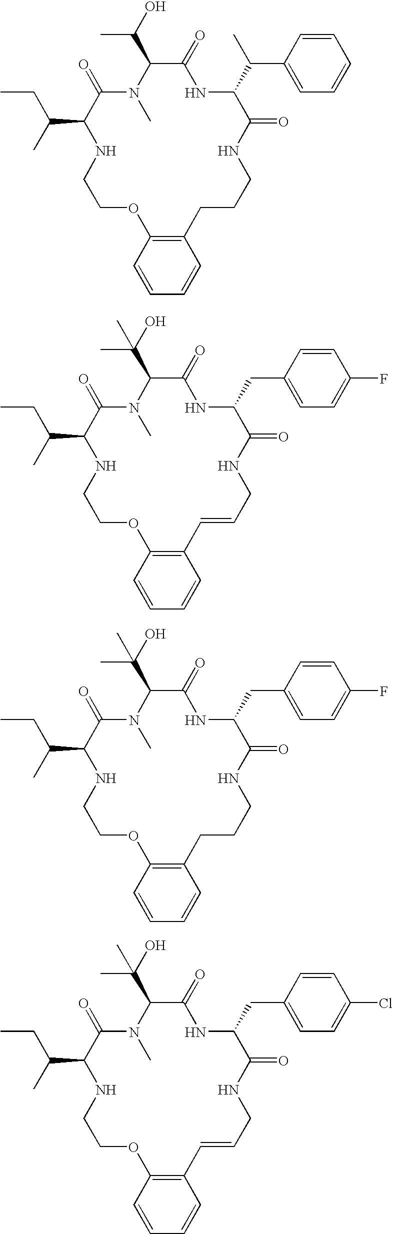 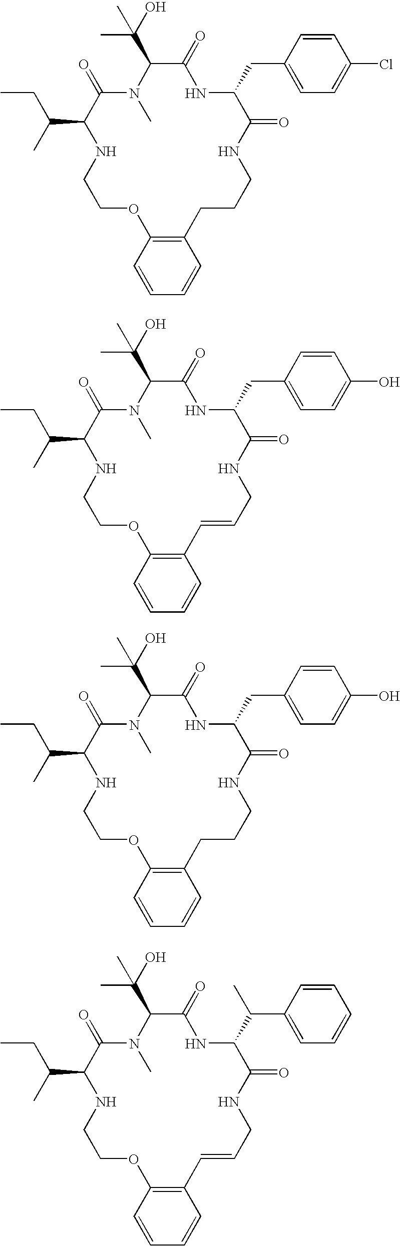 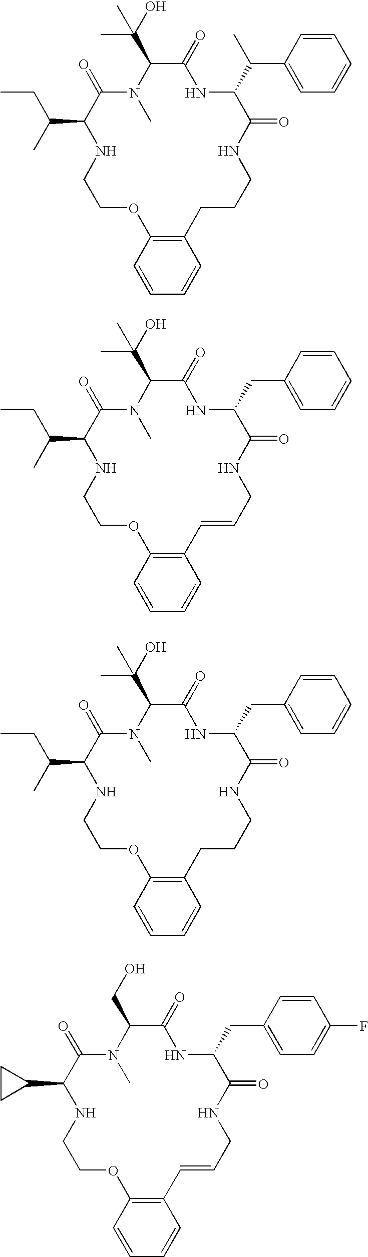 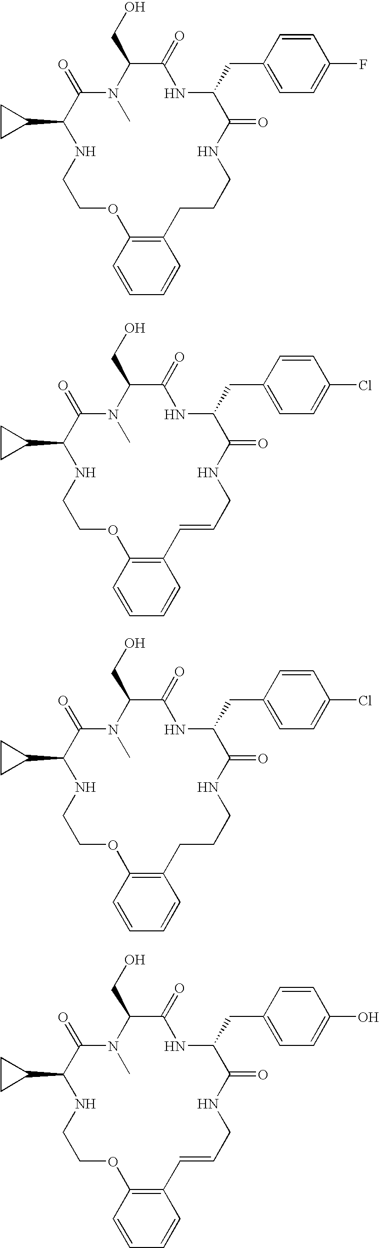 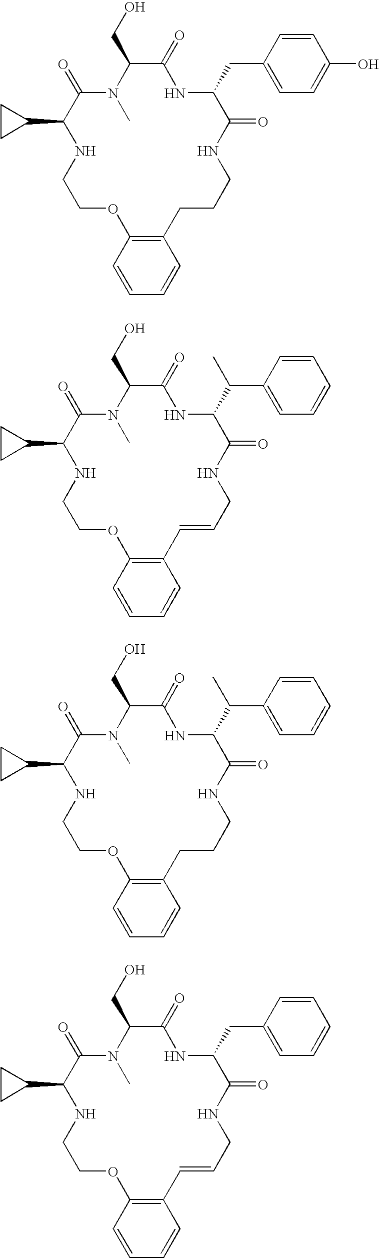 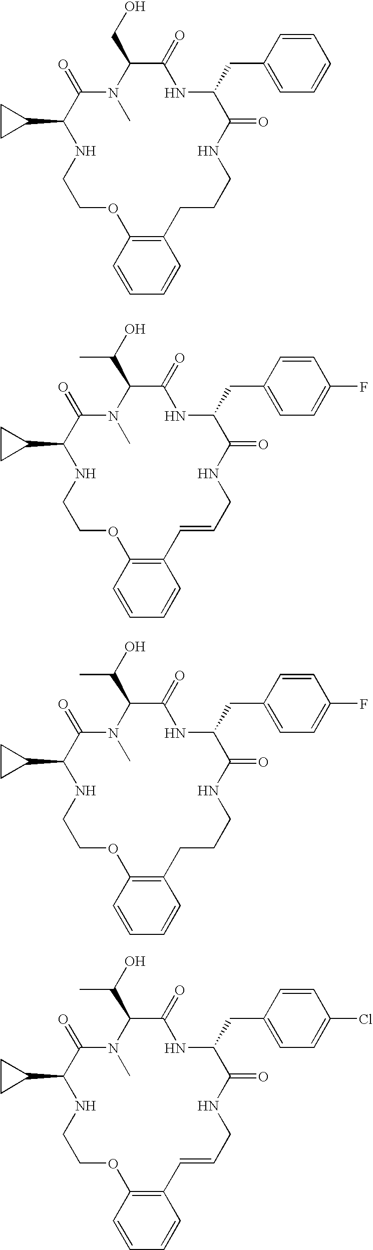 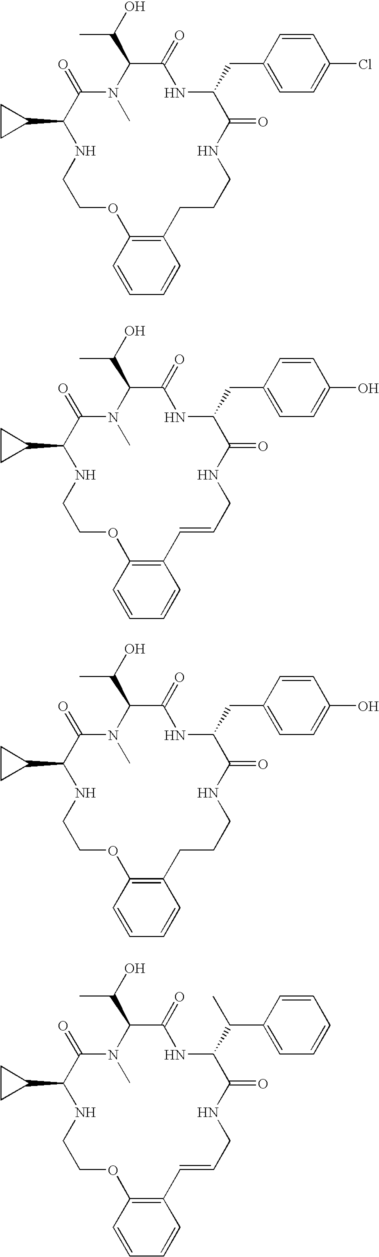 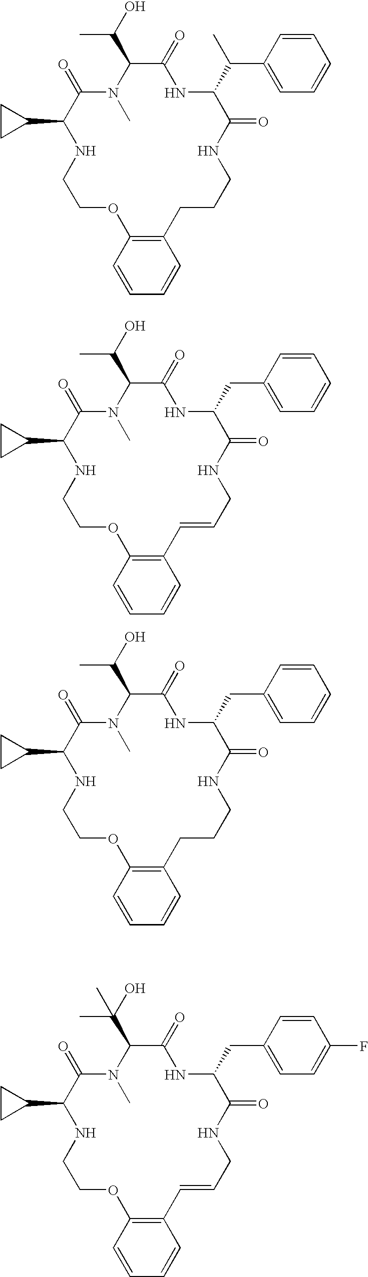 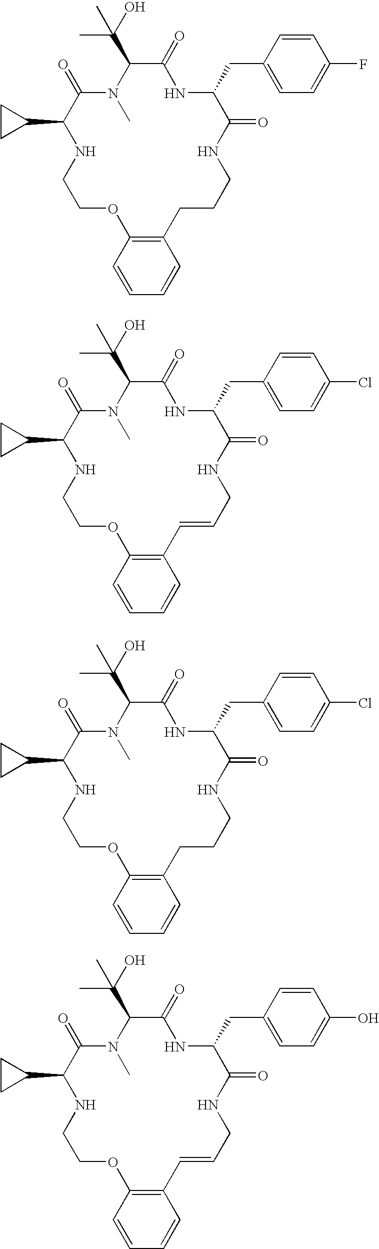 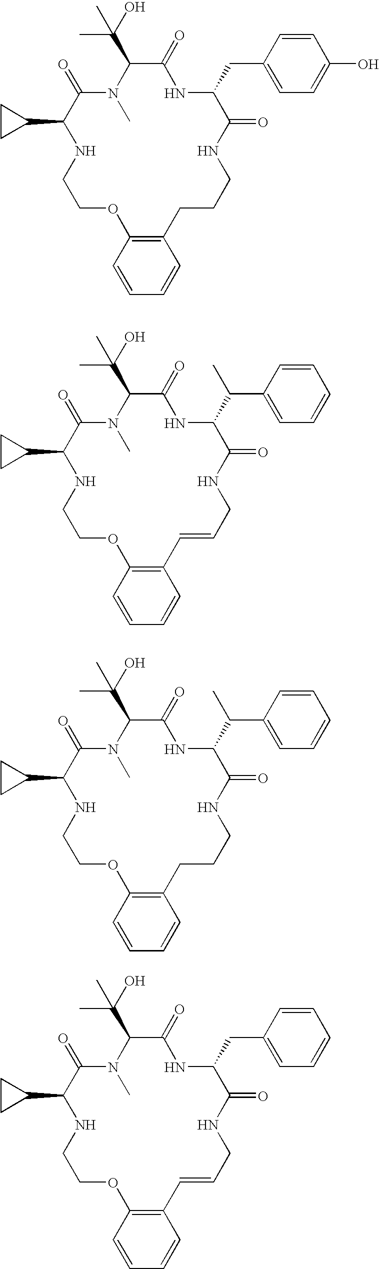 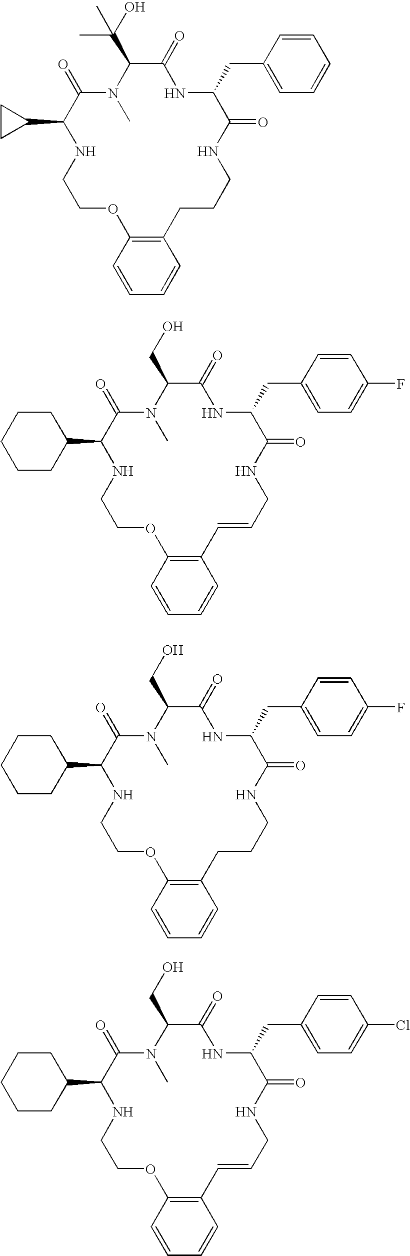 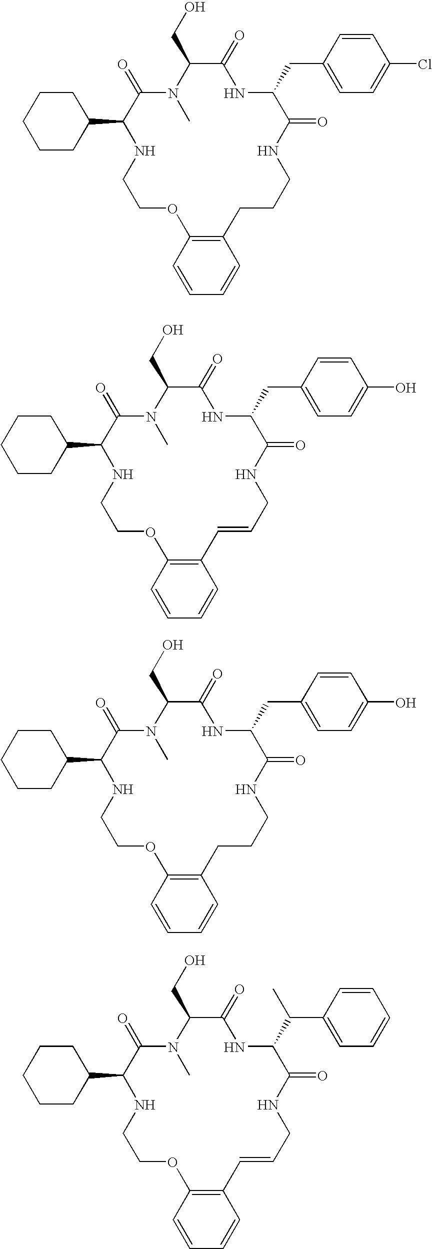 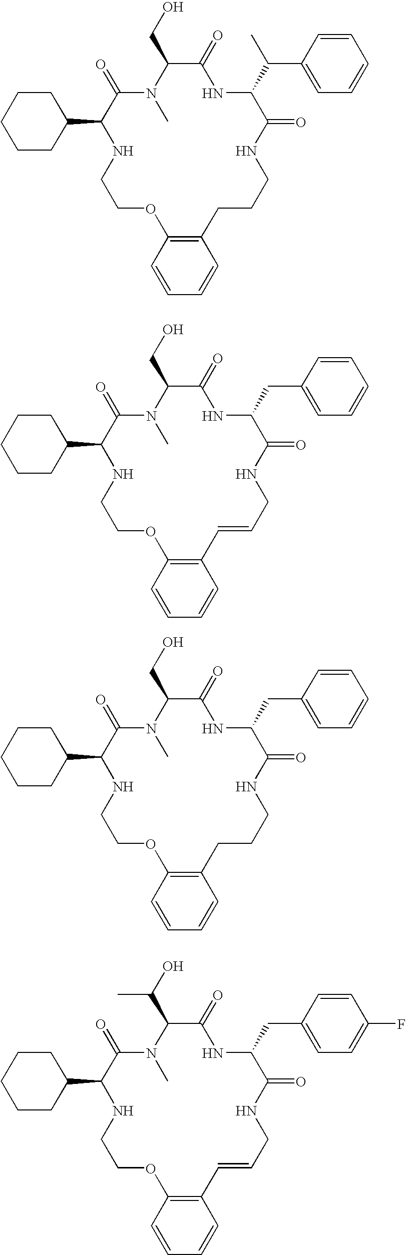 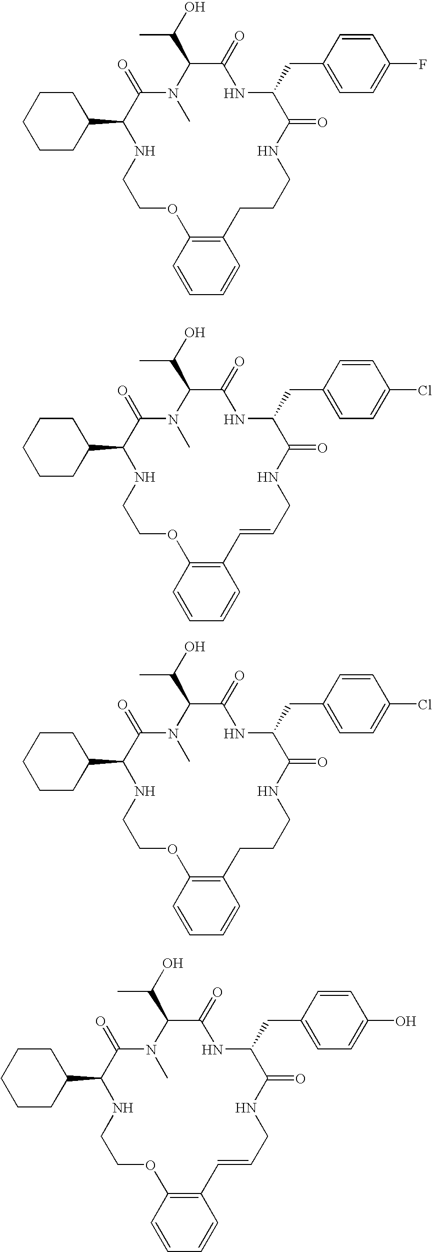 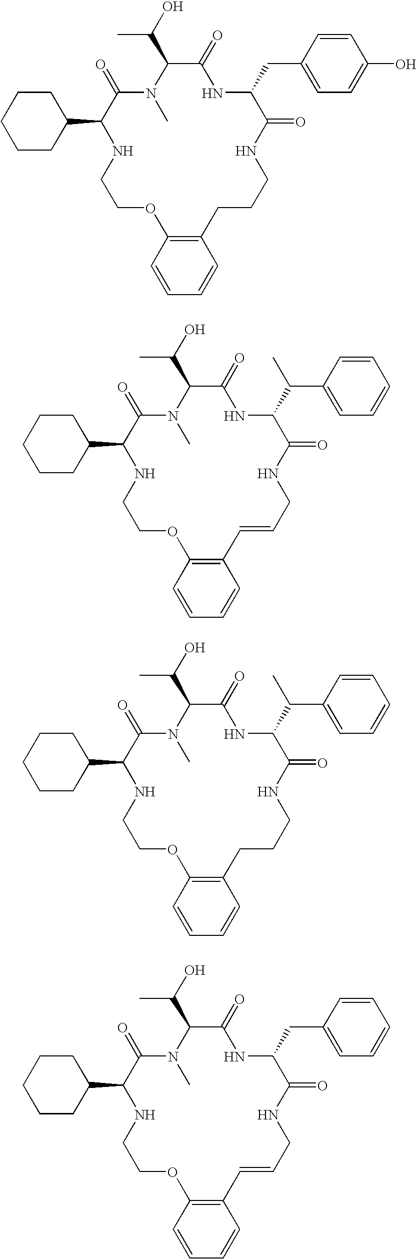 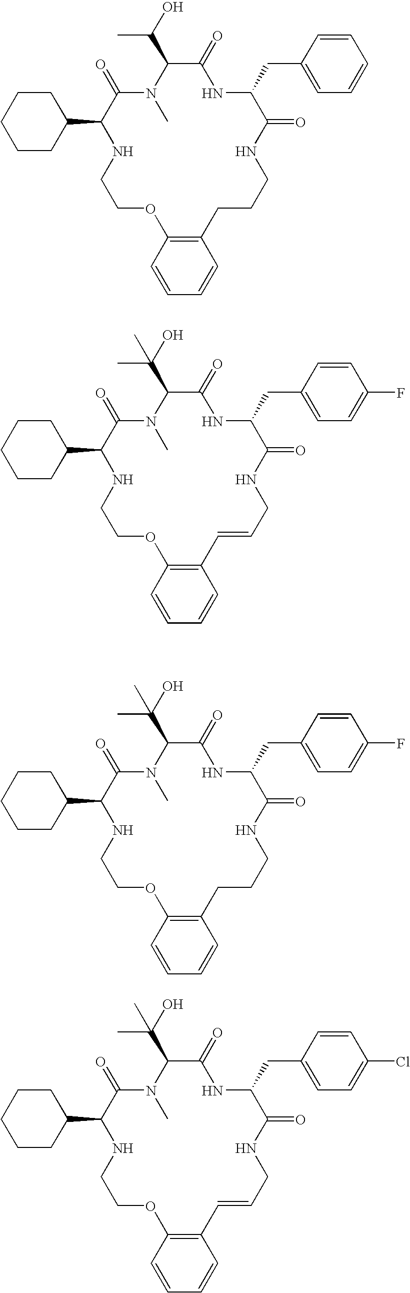 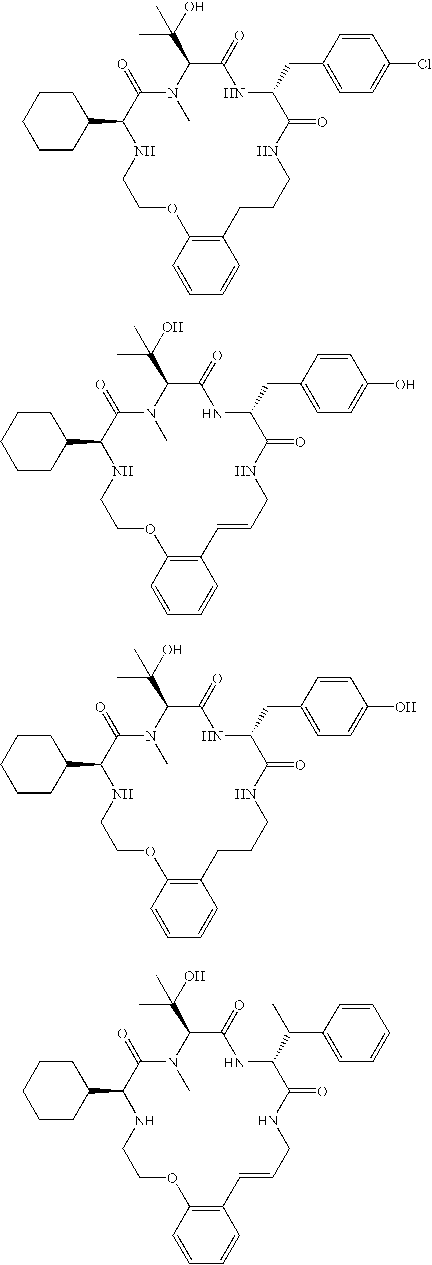 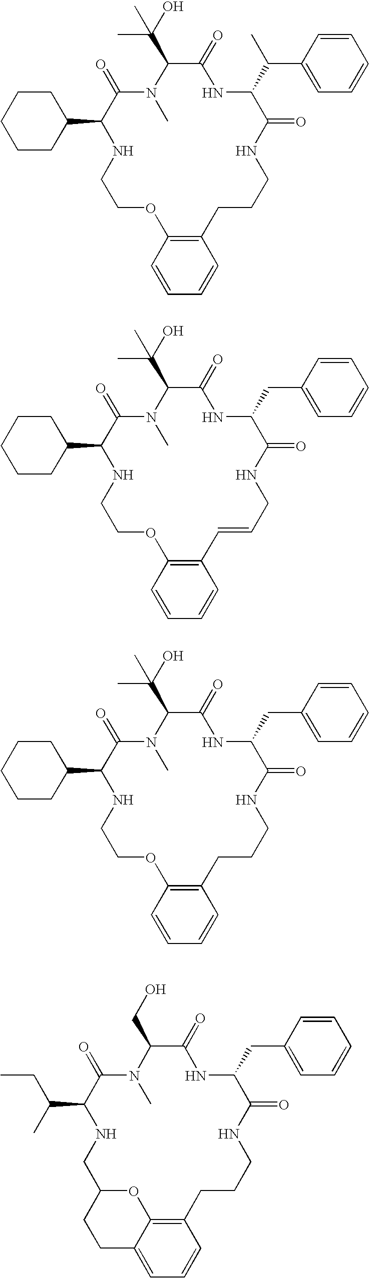 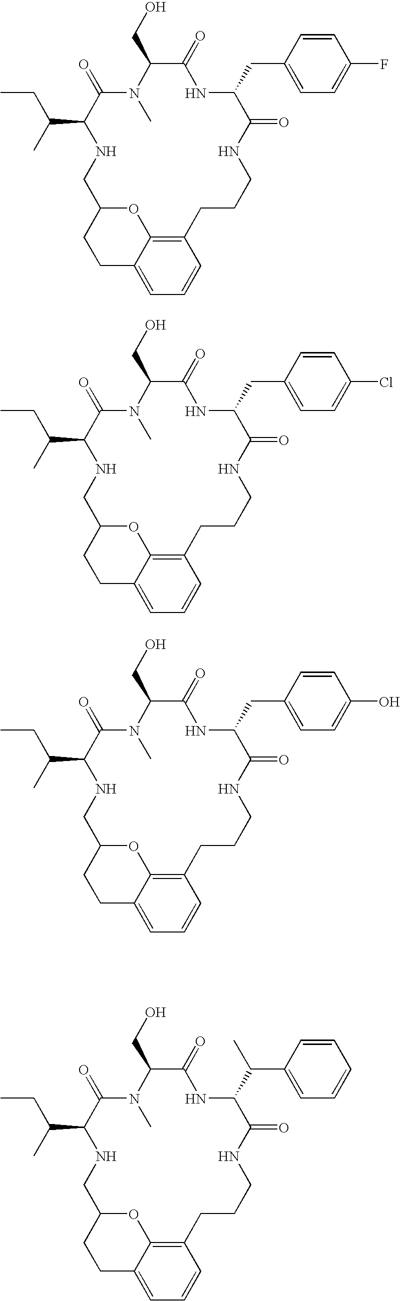 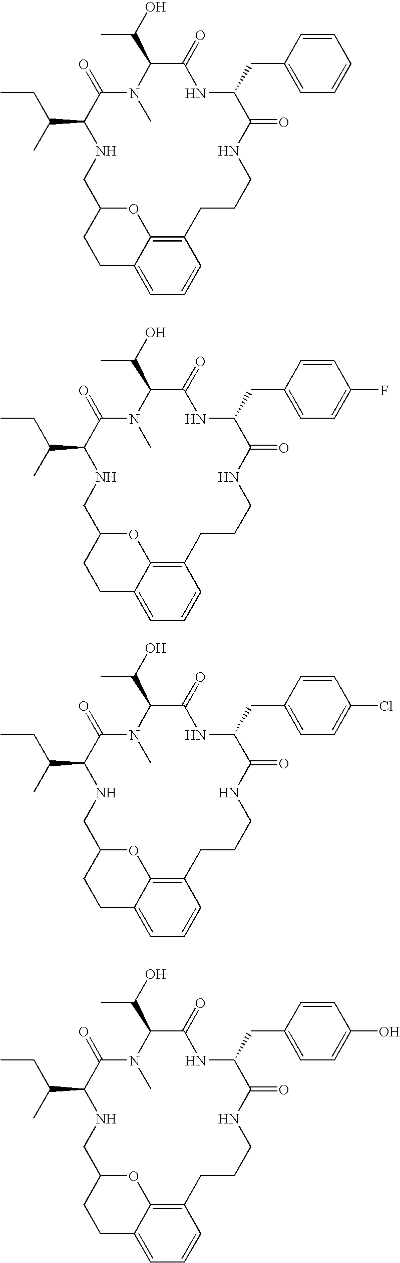 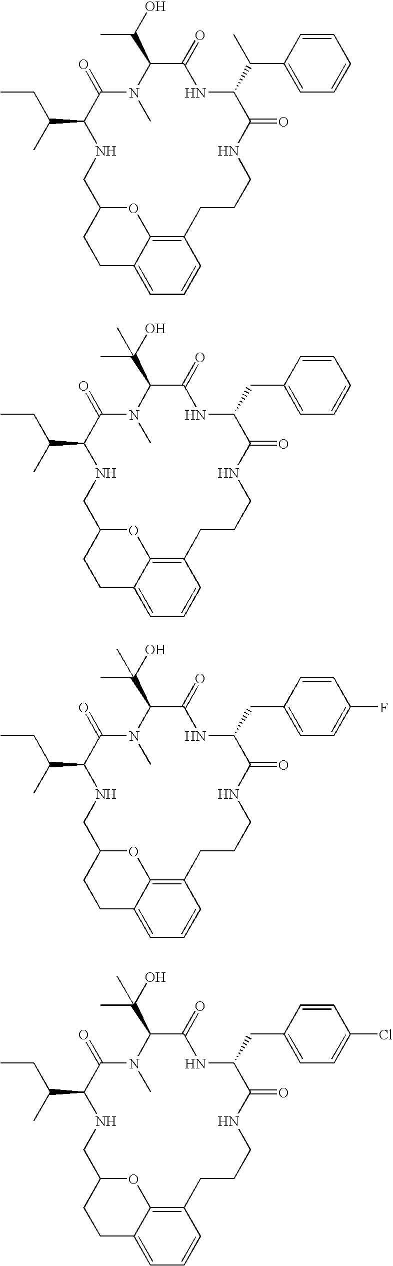 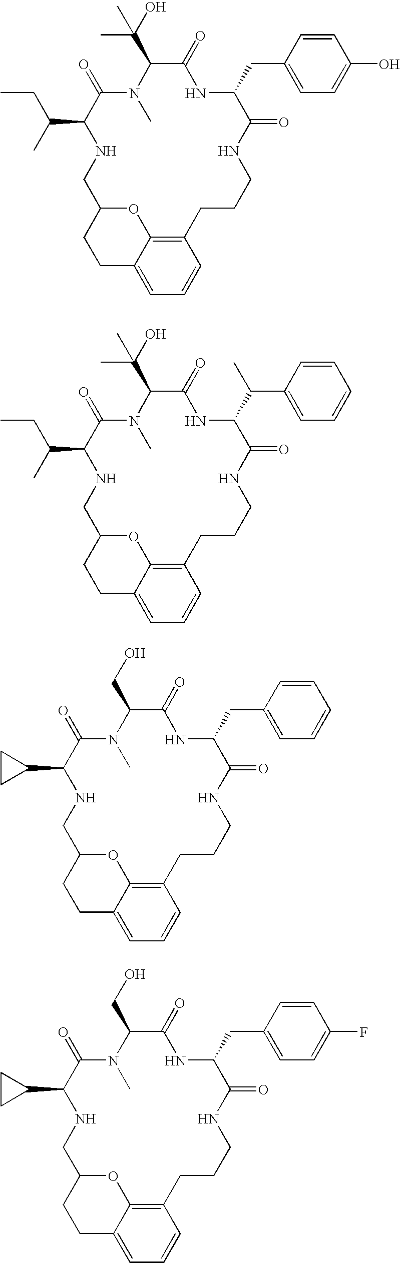 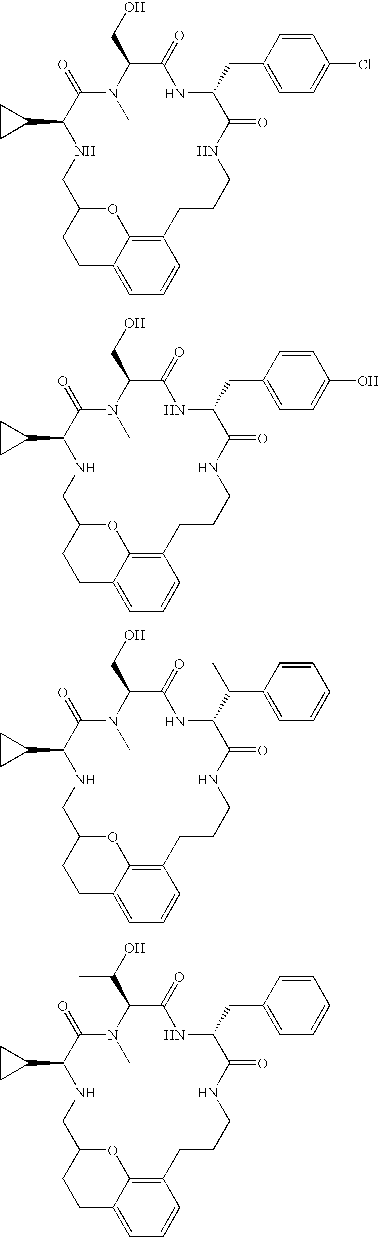 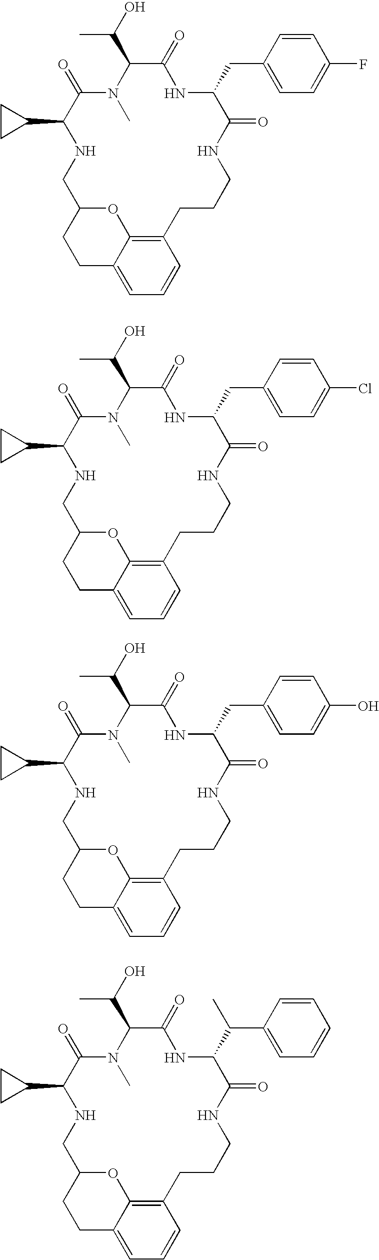 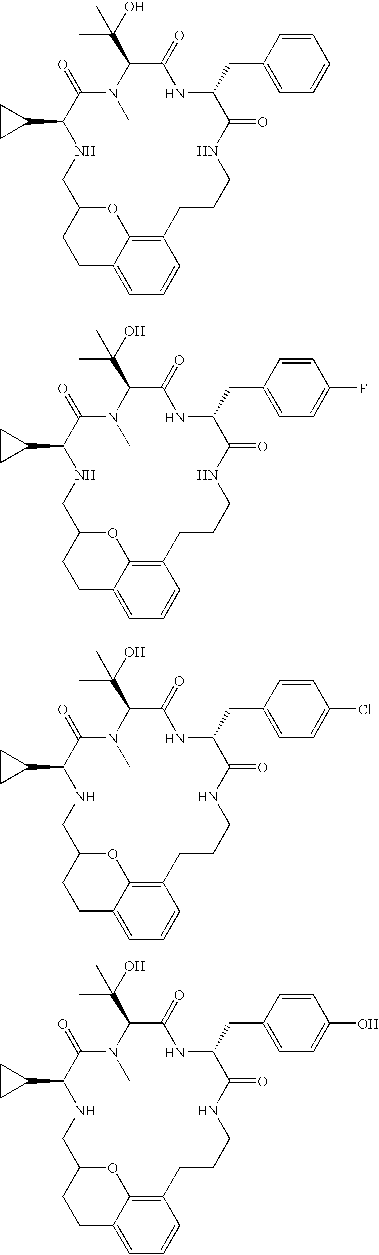 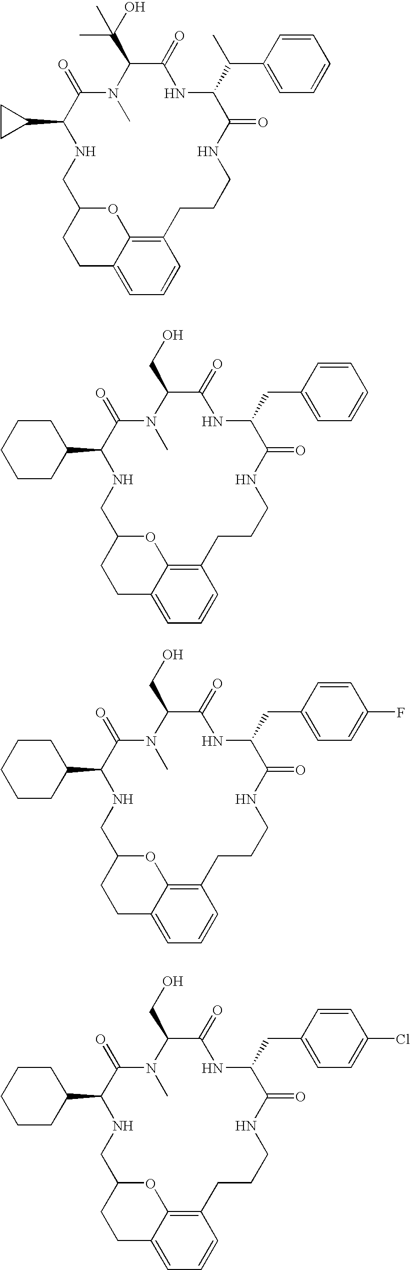 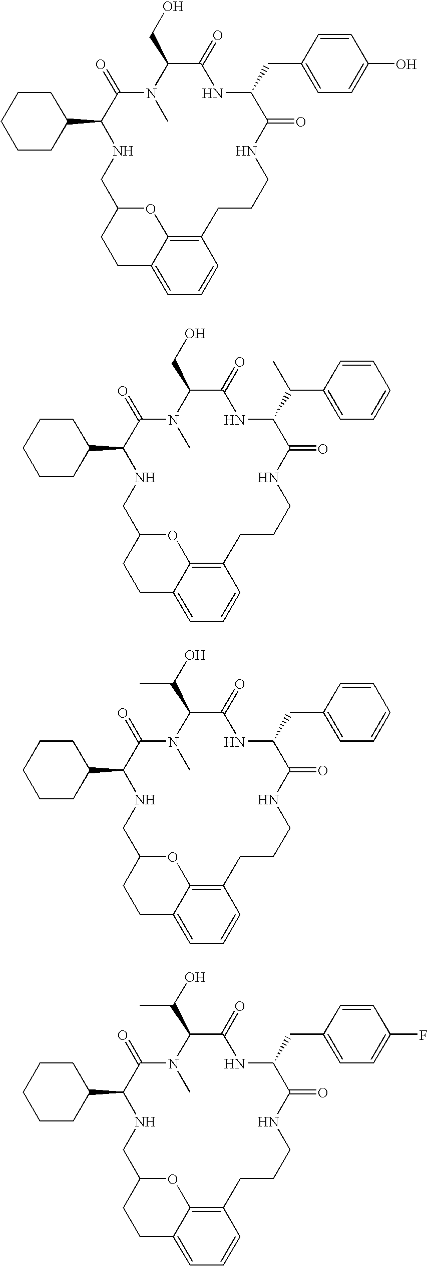 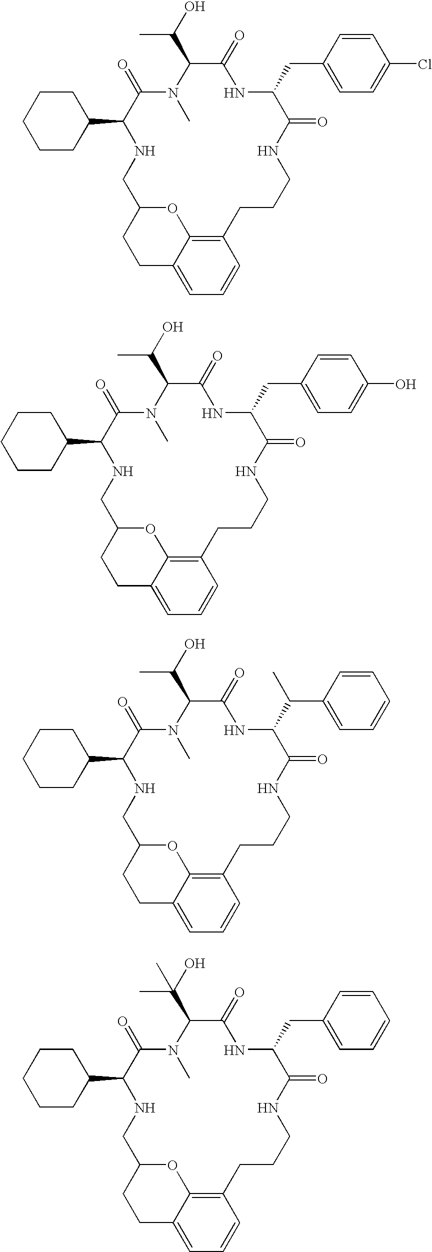 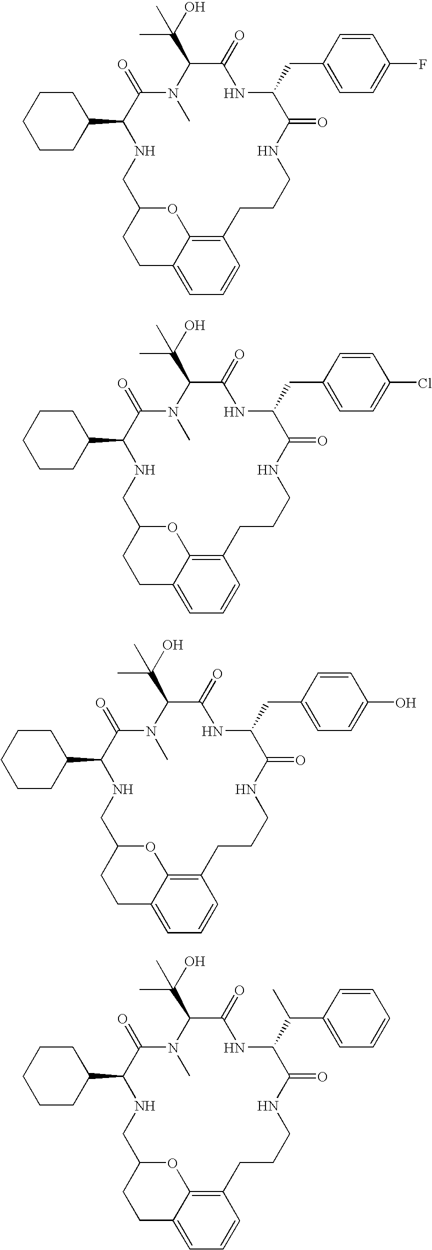
or
an optical isomer, enantiomer, diastereomer, racemate or stereochemical mixture thereof, wherein the container is packaged with optional instructions for the use thereof.

8. A method of modulating GHS-R1a receptor activity in a mammal comprising administering to said mammal an effective GHS-R1a receptor activity modulating amount of a compound of claim 1.

9. The method of claim 8, wherein administering the effective GHS-R1a receptor activity modulating amount of compound I does not result in a significant amount of growth hormone release.

10. The method of claim 8, wherein the compound is a ghrelin receptor antagonist.

11. The method of claim 8, wherein the compound is a GHS-R1a receptor antagonist.

12. The method of claim 8, wherein the compound is a ghrelin receptor inverse agonist.

13. The method of claim 8, wherein the compound is a ghrelin receptor antagonist and a ghrelin receptor inverse agonist.

14. A method of treating a metabolic and/or endocrine disorder comprising administering to a subject in need thereof an effective amount of a compound of claim 1.

15. The method of claim 14, wherein the metabolic and/or endocrine disorder is obesity or an obesity-associated condition.

16. The method of claim 14, wherein the metabolic and/or endocrine disorder is type II diabetes.

17. A method of treating a metabolic and/or endocrine disorder comprising administering to a subject in need thereof an effective amount of a compound of claim 4.

18. A method of treating an inflammatory disorder comprising administering to a subject in need thereof an effective amount of a compound of claim 1.

19. A method of treating a cardiovascular disease comprising administering to a subject in need thereof an effective amount of a compound of claim 1.

20. A method of treating a hyperproliferative disorder comprising administering to a subject in need thereof an effective amount of a compound of claim 1.

21. A method of treating an appetite or eating disorder comprising administering to a subject in need thereof an effective amount of a compound of claim 1.

22. A method of treating Prader-Willi syndrome comprising administering to a subject in need thereof an effective amount of a compound of claim 1.

23. A method of treating a gastrointestinal disorder comprising administering to a subject in need thereof an effective amount of a compound of claim 1.

24. A method of treating liver disease comprising administering to a subject in need thereof an effective amount of a compound of claim 1.

25. The method of claim 24, wherein the liver disease is cirrhosis or chronic liver disease.

26. A method of treating hyperphagia comprising administering to a subject in need thereof an effective amount of a compound of claim 1.

27. The method of claim 26, wherein the hyperphagia is diabetic hyperphagia.
